SEQUENCE LISTING (24 sequences)

SEQ ID NO: 1 - DNA, 23 bp, Canis lupus familiaris
ctgctatgcgcctttgctcccgg

SEQ ID NO: 2 - DNA, 23 bp, Homo Sapiens
atgttttgcacttttgcccttgg

SEQ ID NO: 3 - DNA, 23 bp, Pan troglodytes
atgttttgcacttttgcccttgg

SEQ ID NO: 4 - DNA, 23 bp, Gorilla gorilla gorilla
atgttttgcacttttgcccttgg

SEQ ID NO: 5 - DNA, 23 bp, Pongo abelii
atgttttgcacttttgcccttgg

SEQ ID NO: 6 - DNA, 23 bp, Macaca mulatta
atattttgcacttttgcccttgg

SEQ ID NO: 7 - DNA, 23 bp, Papio hamadryas
atattttgcacttttgcccttgg

SEQ ID NO: 8 - DNA, 23 bp, Callithrix jacchus
atgttgtgcacttttgcccttgg

SEQ ID NO: 9 - DNA, 23 bp, Tarsius syrichta
atgttttgcacttttgcccttgg

SEQ ID NO: 10 - DNA, 23 bp, Microcebus murinus
atgttatgtgcttttgcactcag

SEQ ID NO: 11 - DNA, 23 bp, Mus musculus
acattttacacttttgccctcag

SEQ ID NO: 12 - DNA, 23 bp, Dipodomys ordii
atgttttttgcttttgccctgag

SEQ ID NO: 13 - DNA, 23 bp, Cavia porcellus
atgttttgcacttttgccctcag

SEQ ID NO: 14 - DNA, 23 bp, Oryctolagus cuniculus
acattttgcactttcatcctcgg

SEQ ID NO: 15 - DNA, 23 bp, Ochotona princeps
atgttttatacttttgtccttgg

SEQ ID NO: 16 - DNA, 23 bp, Vicugna pacos
atgttttgtgcttttgccttcag

SEQ ID NO: 17 - DNA, 23 bp, Tursiops truncatus
atgctttgcacctttgccttcag

SEQ ID NO: 18 - DNA, 23 bp, Bos taurus
atgttttgtccttttgccttcag

SEQ ID NO: 19 - DNA, 23 bp, Felis catus
ctgttttgcacttttgcccctgg

SEQ ID NO: 20 - DNA, 22 bp, Myotis lucifugus
gtgttttacactttgtccttgg

SEQ ID NO: 21 - DNA, 23 bp, Pteropus vampyrus
atgttttgtacttttgctcttgg

SEQ ID NO: 22 - DNA, 23 bp, Loxodonta africana
aggttttgtatttttactctcag

SEQ ID NO: 23 - DNA, 23 bp, Procavia capensis
aggtttcatacgtttgctatcag

SEQ ID NO: 24 - DNA, 11 bp, Artificial sequence
  cis motif
gcgnnanttcc

(19) United States
(12) Patent Application Publication
(10) Pub. No.: US 2016/0024588 A1
(43) Pub. Date: Jan. 28, 2016

(54) OSTEOSARCOMA-ASSOCIATED RISK MARKERS AND USES THEREOF

(71) Applicants: Snaevar SIGURDSSON, (US); Emma IVANSSON, (US); THE BROAD INSTITUTE, INC., Cambridge, MA (US); PRESIDENT AND FELLOWS OF HARVARD COLLEGE, Cambridge, MA (US)

(72) Inventors: Elinor Karlsson, Cambridge, MA (US); Kerstin Lindblad-Toh, Malden, MA (US); Snaevar Sigurdsson, Gardabaer (IE); Emma Ivansson, Storvreta (SE)

(73) Assignees: The Broad Institute, Inc., Cambridge, MA (US); President and Fellows of Harvard College, Cambridge, MA (US)

(21) Appl. No.: 14/774,797

(22) PCT Filed: Mar. 14, 2014
(86) PCT No.: PCT/US14/27247

§ 371 Date: Sep. 11, 2015

Related U.S. Application Data
(60) Provisional application No. 61/785,051, filed on Mar. 14, 2013.

(51) Int. Cl. C12Q 1/68

(52) U.S. Cl. CPC C12Q 1/6886; C12Q 2600/158; C12Q 2600/156

(57) ABSTRACT

Provided herein are methods and compositions for identifying subjects, including canine subjects, as having an elevated risk of developing cancer or having an undiagnosed osteosarcoma. These subjects are identified based on the presence of germ-line risk markers.

### CROSS-REFERENCE TO RELATED APPLICATIONS

This application claims the benefit of the filing date of U.S. Provisional Application No. 61/785,051, filed Mar. 14, 2013, the entire contents of which are incorporated by reference herein.

### FEDERALLY SPONSORED RESEARCH OR DEVELOPMENT

This invention was made with U.S. Government support under U54 HG003067 awarded by the National Institutes of Health. The U.S. Government has certain rights in the invention. The research was also generously supported and funded by the Swedish government and Uppsala University.

### BACKGROUND OF INVENTION

Osteosarcoma, a common bone malignancy, is an aggressive cancer characterized by early metastasis and high mortality. In dogs, osteosarcoma typically afflicts middle-age large and giant breeds. Osteosarcoma is common in both humans and dogs resulting in a major impact on human and canine health.

### SUMMARY OF INVENTION

The invention is premised on the identification of germ-line risk markers (e.g., SNPs) that can be used singly or together (e.g., forming a haplotype) to predict elevated risk of osteosarcoma in subjects, e.g., canine subjects. As described herein, a genome-wide association study (GWAS) was performed in Greyhounds, Rottweilers and Irish wolfhounds and germ-line risk markers that correlate with canine osteosarcoma were identified. These germ-line risk markers were confirmed to correlate with canine osteosarcoma in a second, larger sample set. Accordingly, aspects of the invention provide methods for identifying subjects that are at elevated risk of developing osteosarcoma or subjects having otherwise undiagnosed osteosarcoma. Subjects are identified based on the presence of one or more germ-line risk markers shown to be associated with the presence of osteosarcoma, in accordance with the invention. Prognostic and theranostic methods utilizing one or more germ-line risk markers are also described herein.

In some aspects, the disclosure relates to a method, comprising a) analyzing genomic DNA from a canine subject for the presence of a single nucleotide polymorphism (SNP) selected from:
i) one or more chromosome 1 SNPs,
ii) one or more chromosome 2 SNPs,
iii) one or more chromosome 3 SNPs,
iv) one or more chromosome 5 SNPs,
v) one or more chromosome 7 SNPs,
vi) one or more chromosome 8 SNPs,
vii) one or more chromosome 9 SNPs,
viii) one or more chromosome 11 SNPs,
ix) one or more chromosome 13 SNPs,
x) one or more chromosome 14 SNPs,
xi) one or more chromosome 15 SNPs,
xii) one or more chromosome 16 SNPs,
xii) one or more chromosome 17 SNPs,
xiv) one or more chromosome 18 SNPs,
xv) one or more chromosome 19 SNPs,
xvi) one or more chromosome 21 SNPs,
xvii) one or more chromosome 25 SNPs,
xvii) one or more chromosome 26 SNPs,
xix) one or more chromosome 32 SNPs,
xx) one or more chromosome 35 SNPs,
xxi) one or more chromosome 36 SNPs, and
xxii) one or more chromosome 38 SNPs; and
   b) identifying a canine subject having the SNP as a subject at elevated risk of developing osteosarcoma or having an undiagnosed osteosarcoma.

In some embodiments, the SNP is selected from BICF2P133066, BICF2P1421479, BICF2S2308696, BICF2P508906, BICF2P508905, BICF2S23216058, BICF2S23216058, BICF2P266591, BICF2P1332375, BICF2S23231062, BICF2S22945043, BICF2P326880, BICF2P893664, BICF2P1420547, BICF2P698281, BICF2S22919383, BICF2S22947803, BICF2S22947803, BICF2S22959094, BICF2S23228287, BICF2S23036972, BICF2P51623, BICF2P1346510, BICF2P1323908, BICF2P1137984, BICF2P1115364, BICF2P58266, BICF2P627162, BICF2P1422910, BICF2P162782, BICF2P162782, BICF2P1342901, BICF2P868731, BICF2P768889, BICF2P1052528, BICF2P408119, BICF2P1468011, BICF2P219326, BICF2P1462759, BICF2P307386, BICF2P1010170, BICF2S23038485, BICF2G630672865, BICF2G630672813, BICF2P1369145, BICF2G630672770, BICF2P81989, BICF2P916235, BICF2G630672753, BICF2P1177075, BICF2P411325, BICF2P1210630, TIGRP2P407733, BICF2P341331, BICF2P318350, BICF2S2335735, BICF2P1003572, BICF2P1104551, BICF2S23550277, BICF2P870378, BICF2P866460, BICF2P1303772, BICF2S23738710, BICF2P344455, BICF2P825177, BICF2S23324500, BICF2S23544574, BICF2P119783, BICF2S23758510, BICF2S23724888, BICF2P1129874, BICF2S23535303, BICF2S23520119, G326F32S322, BICF2S23238674, BICF2P645758, BICF2P189890, BICF2P819174, BICF2P162666, BICF2P1366853, BICF2P775251, BICF2S23746532, BICF2P1162557, BICF2S23538747, BICF2S23538670, BICF2S23218055, BICF2P680751, BICF2S23510137, BICF2P849639, BICF2S22945333, BICF2S2298851, TIGRP2P238123, TIGRP2P238132, BICF2P1466354, BICF2P440326, BICF2P874005, BICF2P928021, BICF2P1182592, BICF2P1378069, TIGRP2P238162, TIGRP2P253880, BICF2P461252, BICF2P879737, BICF2P163146, BICF2S23259485, TIGRP2P253975, BICF2S23760612, TIGRP2P254013, TIGRP2P254028, BICF2S23750273, BICF2P228579, TIGRP2P254054, BICF2P531896, TIGRP2P254060, BICF2P766570, BICF2P1014267, BICF2P1006929, BICF2P1299781, BICF2P672676, BICF2S23761559, BICF2P15617, BICF2P439160, TIGRP2P254095, TIGRP2P254109, BICF2P477812, BICF2P1238318, BICF2P1354921, BICF2S23741435, BICF2P37118, TIGRP2P254175, BICF2P1123483, TIGRP2P254184, BICF2P825842, BICF2P243632, BICF2P1139856, BICF2P1376844, TIGRP2P254212, TIGRP2P254216, and TIGRP2P254223.

In some embodiments, the SNP is selected from BICF2P133066, BICF2S2308696, BICF2P508906, BICF2P508905, BICF2S23216058, BICF2S23216058, BICF2P266591, BICF2P1332375, BICF2S23231062, BICF2S22945043, BICF2P326880, BICF2P893664, BICF2P1420547, BICF2P698281, BICF2S22919383, BICF2S22947803, BICF2S22947803, BICF2S22959094, BICF2S23228287, BICF2S23036972, BICF2P51623, BICF2P1346510, BICF2P1323908, BICF2P1137984, BICF2P1115364, BICF2P58266, BICF2P627162, BICF2P1422910, BICF2P162782, BICF2P162782, BICF2P1342901, BICF2P868731, BICF2P768889, BICF2P1052528, BICF2P408119, BICF2P1468011, BICF2P219326, BICF2P1462759, BICF2P307386, BICF2P1010170, BICF2P229090, BICF2S23516022, and BICF2S22922837. In some embodiments, the SNP is BICF2P133066.

In some embodiments, the SNP is two or more SNPs. In some embodiments, the SNP is three or more SNPs.

Other aspects of the disclosure relate to a method, comprising (a) analyzing genomic DNA from a canine subject for the presence of a risk haplotype selected from:

a risk haplotype having chromosome coordinates chr11:44392734-44414985,

a risk haplotype having chromosome coordinates chr8:35433142-35454649,

a risk haplotype having chromosome coordinates chr13:14549973-14645634,

a risk haplotype having chromosome coordinates chr25:21831580-21921256,

a risk haplotype having chromosome coordinates chr14:48831824-49203827,

a risk haplotype having chromosome coordinates chr5:16071171-16152955,

a risk haplotype having chromosome coordinates chr19:33963105-34145310,

a risk haplotype having chromosome coordinates chr16:43665149-43737129,

a risk haplotype having chromosome coordinates chr15:63767963-63800415,

a risk haplotype having chromosome coordinates chr16:40883517-41081510,

a risk haplotype having chromosome coordinates chr25:43476429-43528145,

a risk haplotype having chromosome coordinates chr1:112977233-113081800,

a risk haplotype having chromosome coordinates chr3:5162058-6465753,

a risk haplotype having chromosome coordinates chr7:64631053-64703475,

a risk haplotype having chromosome coordinates chr1:115582915-116790630,

a risk haplotype having chromosome coordinates chr2:19212450-19542015,

a risk haplotype having chromosome coordinates chr1:122033806-122051988,

a risk haplotype having chromosome coordinates chr35:18326079-18345318,

a risk haplotype having chromosome coordinates chr9:47647012-47668054,

a risk haplotype having chromosome coordinates chr38:11252518-11739329,

a risk haplotype having chromosome coordinates chr21:46231985-46363479,

a risk haplotype having chromosome coordinates chr17:14465884-14482152,

a risk haplotype having chromosome coordinates chr32:25136302-25156153,

a risk haplotype having chromosome coordinates chr36:29637804-29663408,

a risk haplotype having chromosome coordinates chr15:37986345-39974762,

a risk haplotype having chromosome coordinates chr1:29405587-29914411,

a risk haplotype having chromosome coordinates chr26:32374093-32428448,

a risk haplotype having chromosome coordinates chr25:29658978-29767164,

a risk haplotype having chromosome coordinates chr26:3529343-3550075,

a risk haplotype having chromosome coordinates chr5:14720254-15466603,

a risk haplotype having chromosome coordinates chr18:4266743-5854451,

a risk haplotype having chromosome coordinates chr1:16768869-18150476,

a risk haplotype having chromosome coordinates chr9:18896060-19633155, and

a risk haplotype having chromosome coordinates chr11:44390633-44406002; and

(b) identifying a canine subject having the mutation as a subject at elevated risk of developing osteosarcoma or having an undiagnosed osteosarcoma.

In some embodiments, the risk haplotype is selected from a risk haplotype having chromosome coordinates chr11:44392734-44414985, a risk haplotype having chromosome coordinates chr8:35433142-35454649, a risk haplotype having chromosome coordinates chr1:115582915-116790630, a risk haplotype having chromosome coordinates chr2:19212450-19542015, a risk haplotype having chromosome coordinates chr1:122033806-122051988, a risk haplotype having chromosome coordinates chr35:18326079-18345318, a risk haplotype having chromosome coordinates chr9:47647012-47668054, a risk haplotype having chromosome coordinates chr38:11252518-11739329, a risk haplotype having chromosome coordinates chr5:14720254-15466603, and a risk haplotype having chromosome coordinates chr18:4266743-5854451. In some embodiments, the risk haplotype is selected from a risk haplotype having chromosome coordinates chr11:44392734-44414985, a risk haplotype having chromosome coordinates chr1:115582915-116790630, and a risk haplotype having chromosome coordinates chr5:14720254-15466603. In some embodiments, the risk haplotype is the risk haplotype having chromosome coordinates chr11:44392734-44414985.

In some embodiments, the mutation is two or more mutations. In some embodiments, the mutation is three or more mutations. In some embodiments, the genomic region is two or more genomic regions. In some embodiments, the genomic region is three or more genomic regions.

In yet another aspect, the disclosure relates to a method, comprising (a) analyzing genomic DNA from a canine subject for the presence of a mutation in a gene selected from:

one or more genes located within a risk haplotype having chromosome coordinates chr11:44392734-44414985,

one or more genes located within a risk haplotype having chromosome coordinates chr8:35433142-35454649,

one or more genes located within a risk haplotype having chromosome coordinates chr13: 14549973-14645634,

one or more genes located within a risk haplotype having chromosome coordinates chr25:21831580-21921256,

one or more genes located within a risk haplotype having chromosome coordinates chr14:48831824-49203827,

one or more genes located within a risk haplotype having chromosome coordinates chr5:16071171-16152955,

one or more genes located within a risk haplotype having chromosome coordinates chr19:33963105-34145310,

one or more genes located within a risk haplotype having chromosome coordinates chr16:43665149-43737129,

one or more genes located within a risk haplotype having chromosome coordinates chr15:63767963-63800415,

one or more genes located within a risk haplotype having chromosome coordinates chr16:40883517-41081510,

one or more genes located within a risk haplotype having chromosome coordinates chr25:43476429-43528145,

one or more genes located within a risk haplotype having chromosome coordinates chr1:112977233-113081800,

one or more genes located within a risk haplotype having chromosome coordinates chr3:5162058-6465753,

one or more genes located within a risk haplotype having chromosome coordinates chr7:64631053-64703475,

one or more genes located within a risk haplotype having chromosome coordinates chr1:115582915-116790630,

one or more genes located within a risk haplotype having chromosome coordinates chr2:19212450-19542015,

one or more genes located within a risk haplotype having chromosome coordinates chr1:122033806-122051988,

one or more genes located within a risk haplotype having chromosome coordinates chr35: 18326079-18345318,

one or more genes located within a risk haplotype having chromosome coordinates chr9:47647012-47668054,

one or more genes located within a risk haplotype having chromosome coordinates chr38:11252518-11739329,

one or more genes located within a risk haplotype having chromosome coordinates chr21:46231985-46363479,

one or more genes located within a risk haplotype having chromosome coordinates chr17: 14465884-14482152,

one or more genes located within a risk haplotype having chromosome coordinates chr32:25136302-25156153,

one or more genes located within a risk haplotype having chromosome coordinates chr36:29637804-29663408,

one or more genes located within a risk haplotype having chromosome coordinates chr15:37986345-39974762,

one or more genes located within a risk haplotype having chromosome coordinates chr1:29405587-29914411,

one or more genes located within a risk haplotype having chromosome coordinates chr26: 32374093-32428448,

one or more genes located within a risk haplotype having chromosome coordinates chr25:29658978-29767164,

one or more genes located within a risk haplotype having chromosome coordinates chr26:3529343-3550075,

one or more genes located within a risk haplotype having chromosome coordinates chr5:14720254-15466603,

one or more genes located within a risk haplotype having chromosome coordinates chr18:4266743-5854451,

one or more genes located within a risk haplotype having chromosome coordinates chr1:16768869-18150476,
one or more genes located within a risk haplotype having chromosome coordinates chr9:18896060-19633155, and
one or more genes located within a risk haplotype having chromosome coordinates chr11:44390633-44406002; and
   (b) identifying a canine subject having the mutation as a subject at elevated risk of developing osteosarcoma or having an undiagnosed osteosarcoma.

In some embodiments, the gene is selected from one or more genes located within a risk haplotype having chromosome coordinates chr11:44392734-44414985, one or more genes located within a risk haplotype having chromosome coordinates chr8:35433142-35454649, one or more genes located within a risk haplotype having chromosome coordinates chr1:115582915-116790630, one or more genes located within a risk haplotype having chromosome coordinates chr2:19212450-19542015, one or more genes located within a risk haplotype having chromosome coordinates chr1:122033806-122051988, one or more genes located within a risk haplotype having chromosome coordinates chr35:18326079-18345318, one or more genes located within a risk haplotype having chromosome coordinates chr9:47647012-47668054, one or more genes located within a risk haplotype having chromosome coordinates chr38:11252518-11739329, one or more genes located within a risk haplotype having chromosome coordinates chr5:14720254-15466603, and one or more genes located within a risk haplotype having chromosome coordinates chr18:4266743-5854451. In some embodiments, the gene is selected from one or more genes located within a risk haplotype having chromosome coordinates chr11:44392734-44414985, one or more genes located within a risk haplotype having chromosome coordinates chr1:115582915-116790630, and one or more genes located within a risk haplotype having chromosome coordinates chr5:14720254-15466603. In some embodiments, the gene is one or more genes located within the risk haplotype having chromosome coordinates chr11:44392734-44414985.

In some embodiments, the gene is selected from CDKN2B-AS, OTX2, BMPER, GRIK4, EN1, MARCO, MTMR7, SGCZ, CCL20, CD3EAP, ERCC1, ERCC2, FOSB, PPP1R13L, FER, MAN2A1, PJA2, CHST9, ADCK4, AKT2, AXL, BLVRB, C19orf47, C19orf54, CNTD2, CYP2A7, CYP2B6, CYP2S1, DLL3, EGLN2, FBL, FCGBP, GMFG, HIPK4, HNRNPUL1, ITPKC, LEUTX, LTBP4, MAP3K10, MED29, NUMBL, PLD3, PLEKHG2, PSMC4, RAB4B, SAMD4B, SERTAD1, SERTAD3, SHKBP1, SNRPA, SPTBN4, SUPT5H, TIMM50, KIAA1462, C19orf40, CEP89, RHPN2, BLMH, TMIGD1, FAM5C, NELL1, EMCN, AMDHD1, CCDC38, CDK17, ELKS, FGD6, HAL, LTA4H, METAP2, NDUFA12, NEDD1, NR2C1, NTN4, SNRPF, USP44,VEZT, EYA4, TCF21, ARVCF, C22orf25, COMT, XKR6, FBRSL1, BLID, C7orf72, COBL, DDC, FIGNL1, GRB10, IKZF1, VWC2, ZPBP, BCL2, KIAA1468, PHLPP1, PIGN, RNF152, TNFRSF11A, ZCCHC2, ABCA5, KCNJ16, KCNJ2, MAP2K6, CDKN2A, and CDKN2B. In some embodiments, the gene is selected from CDKN2B-AS, OTX2, BMPER, EN1, DLL3, KIAA1462, FAM5C, NELL1, EMCN, TCF21, BLID, VWC2, BCL2, and TNFRSF11A. In some embodiments, the gene is selected from CDKN2B-AS, OTX2, ADCK4, AKT2, AXL, BLVRB, C19orf47, C19orf54, CNTD2, CYP2A7, CYP2B6, CYP2S1, DLL3, EGLN2, FBL, FCGBP, GMFG, HIPK4, HNRNPUL1, ITPKC, LEUTX, LTBP4, MAP3K10, MED29, NUMBL, PLD3, PLEKHG2, PSMC4, RAB4B, SAMD4B, SERTAD1, SERTAD3, SHKBP1, SNRPA, SPTBN4, SUPT5H, TIMM50, KIAA1462, C19orf40, CEP89, RHPN2, BLMH, TMIGD1, FAM5C, BLID, C7orf72, COBL, DDC, FIGNL1, GRB10, IKZF1, VWC2, and ZPBP. In some embodiments, the gene is selected from CDKN2B-AS, ADCK4, AKT2, AXL, BLVRB, C19orf47, C19orf54, CNTD2, CYP2A7, CYP2B6, CYP2S1, DLL3, EGLN2, FBL, FCGBP, GMFG, HIPK4, HNRNPUL1, ITPKC, LEUTX, LTBP4, MAP3K10, MED29, NUMBL, PLD3, PLEKHG2, PSMC4, RAB4B, SAMD4B, SERTAD1, SERTAD3, SHKBP1, SNRPA, SPTBN4, SUPT5H, TIMM50, and BLID. In some embodiments, the gene is selected from CDKN2B-AS, CDKN2A, and CDKN2B.

In some embodiments, the mutation is two or more mutations. In some embodiments, the mutation is three or more mutations. In some embodiments, the gene is two or more genes. In some embodiments, the gene is three or more genes.

In some embodiments of any method provided herein, the genomic DNA is obtained from a bodily fluid or tissue sample of the subject. In some embodiments of any method provided herein, the genomic DNA is obtained from a blood or saliva sample of the subject. In some embodiments of any method provided herein, the genomic DNA is analyzed using a single nucleotide polymorphism (SNP) array. In some embodiments of any method provided herein, the genomic DNA is analyzed using a bead array. In some embodiments of any method provided herein, the genomic DNA is analyzed using a nucleic acid sequencing assay.

In some embodiments of any method described herein, the canine subject is a descendent of a Greyhound, Rottweiler or Irish Wolfhound. In some embodiments, the canine subject is a Greyhound, Rottweiler or Irish Wolfhound.

Yet another aspect of the disclosure relates to a method, comprising (a) analyzing genomic DNA in a sample from a subject for presence of a mutation in a gene selected from:

one or more genes located within a risk haplotype having chromosome coordinates chr11:44392734-44414985 or an orthologue of such a gene,

one or more genes located within a risk haplotype having chromosome coordinates chr8:35433142-35454649 or an orthologue of such a gene,

one or more genes located within a risk haplotype having chromosome coordinates chr13:14549973-14645634 or an orthologue of such a gene,

one or more genes located within a risk haplotype having chromosome coordinates chr25:21831580-21921256 or an orthologue of such a gene,

one or more genes located within a risk haplotype having chromosome coordinates chr14:48831824-49203827 or an orthologue of such a gene,

one or more genes located within a risk haplotype having chromosome coordinates chr5:16071171-16152955 or an orthologue of such a gene,

one or more genes located within a risk haplotype having chromosome coordinates chr19:33963105-34145310 or an orthologue of such a gene,

one or more genes located within a risk haplotype having chromosome coordinates chr16:43665149-43737129 or an orthologue of such a gene,

one or more genes located within a risk haplotype having chromosome coordinates chr15:63767963-63800415 or an orthologue of such a gene,

one or more genes located within a risk haplotype having chromosome coordinates chr16:40883517-41081510 or an orthologue of such a gene,

one or more genes located within a risk haplotype having chromosome coordinates chr25:43476429-43528145 or an orthologue of such a gene,

one or more genes located within a risk haplotype having chromosome coordinates chr1:112977233-113081800 or an orthologue of such a gene,

one or more genes located within a risk haplotype having chromosome coordinates chr3:5162058-6465753 or an orthologue of such a gene,

one or more genes located within a risk haplotype having chromosome coordinates chr7:64631053-64703475 or an orthologue of such a gene,

one or more genes located within a risk haplotype having chromosome coordinates chr1:115582915-116790630 or an orthologue of such a gene,

one or more genes located within a risk haplotype having chromosome coordinates chr2:19212450-19542015 or an orthologue of such a gene,

one or more genes located within a risk haplotype having chromosome coordinates chr1:122033806-122051988 or an orthologue of such a gene,

one or more genes located within a risk haplotype having chromosome coordinates chr35:18326079-18345318 or an orthologue of such a gene,

one or more genes located within a risk haplotype having chromosome coordinates chr9:47647012-47668054 or an orthologue of such a gene,

one or more genes located within a risk haplotype having chromosome coordinates chr38:11252518-11739329 or an orthologue of such a gene,

one or more genes located within a risk haplotype having chromosome coordinates chr21:46231985-46363479 or an orthologue of such a gene,

one or more genes located within a risk haplotype having chromosome coordinates chr17:14465884-14482152 or an orthologue of such a gene,

one or more genes located within a risk haplotype having chromosome coordinates chr32:25136302-25156153 or an orthologue of such a gene,

one or more genes located within a risk haplotype having chromosome coordinates chr36:29637804-29663408 or an orthologue of such a gene,

one or more genes located within a risk haplotype having chromosome coordinates chr15:37986345-39974762 or an orthologue of such a gene,

one or more genes located within a risk haplotype having chromosome coordinates chr1:29405587-29914411 or an orthologue of such a gene,

one or more genes located within a risk haplotype having chromosome coordinates chr26:32374093-32428448 or an orthologue of such a gene,

one or more genes located within a risk haplotype having chromosome coordinates chr25:29658978-29767164 or an orthologue of such a gene,

on or more genes located within a risk haplotype having chromosome coordinates chr26:3529343-3550075 or an orthologue of such a gene,

one or more genes located within a risk haplotype having chromosome coordinates chr5:14720254-15466603 or an orthologue of such a gene,

one or more genes located within a risk haplotype having chromosome coordinates chr18:4266743-5854451 or an orthologue of such a gene,

one or more genes located within a risk haplotype having chromosome coordinates chr1:16768869-18150476 or an orthologue of such a gene,

one or more genes located within a risk haplotype having chromosome coordinates chr9:18896060-19633155 or an orthologue of such a gene, and

one or more genes located within a risk haplotype having chromosome coordinates chr11:44,390,633-44,406,002 or an orthologue of such a gene; and

(b) identifying a subject having the mutation as a subject at elevated risk of developing osteosarcoma or having an undiagnosed osteosarcoma.

In some embodiments, the subject is a human subject. In some embodiments, the subject is a canine subject.

In some embodiments, the genomic DNA is obtained from a bodily fluid or tissue sample of the subject. In some embodiments, the genomic DNA is obtained from a blood or saliva sample of the subject. In some embodiments, the genomic DNA is analyzed using a single nucleotide polymorphism (SNP) array. In some embodiments, the genomic DNA is analyzed using a bead array. In some embodiments, the genomic DNA is analyzed using a nucleic acid sequencing assay.

In some embodiments, the gene is two or more genes. In some embodiments, the gene is three or more genes. In some embodiments, the mutation is two or more mutations. In some embodiments, the mutation is three or more mutations.

### BRIEF DESCRIPTION OF DRAWINGS

FIG. 1 shows results from the genome wide association study (GWAS). (A) A graph showing that each breed clusters as a distinct population. (B) A graph of the inbreeding coefficient for each breed showing that Greyhounds (Greys) are the least inbred, followed by the Rottweilers (Rotts), Irish Wolfhounds (IWHs) and AKC Greys. (C) A graph showing the extent of linkage disequilibrium in each breed. The lines from top to bottom are IWH, Grey AKC, Rott, and Grey. (D) A graph of the regions of homozygosity in each breed. The lines from top to bottom are IWH, Grey AKC, Rott and Grey. (E) A graph of the regions of low relative heterozygosity in each breed. Rott and Grey essentially overlap and are the top two lines. IWH and Grey AKC essentially overlap and are the bottom two lines.

FIG. 2A is a series of graphs showing the significant SNPs identified for each breed across the genome. The approximate boundaries of each chromosome on the X axis are indicated by vertical black lines.

FIG. 2B is a series of graphs showing the variance explained and genotype relative risk for loci with P<0.0005.

FIG. 3 is a series of graphs showing the genome wide association on chromosome 11 region and the syntenic region of human chromosome 9 as well as functional data implicating specific variants as likely disease variants. FIG. 3A is a series of graphs showing the location of CFA11 on dog chromosome 11 and the corresponding syntenic region on human chromosome 9. Blue vertical lines indicate the boundaries of CFA11. Horizontal grey bars show human genomic regions tested for functionality. The bar (G) indicates the human genomic region with the highest expression in a luciferase assay. Several of the most significant SNPs were in high linkage disequilibrium (LD) with the top SNP. FIG. 3B is a graph of luciferase expression driven by human genomic regions A-G in human osteosarcoma cells. FIG. 3C is a diagram showing the location of BICF2P133066 (chr11:44405676) within human genomic region G (vertical lines) and the identity of the nucleotide at the corresponding location in several mammals.

FIG. 4 shows the results of GRAIL (Gene Relationships Across Implicated Loci) analysis used to identify non-random connectivity between genes in the associated loci described herein. The genes associated with each breed as separated by dashed black lines.

FIG. 5 is a series of graphs and tables showing that pathways enriched in GWAS and fixed regions were also enriched for CGH changes in tumors. Numbers indicate the number of genomic loci overlapping between gene set (y axis) and region set (x axis). Those in bold have corrected p<0.05 as calculated by permutation analysis. RRV=regions of reduced variability.

FIG. 6 shows the p-value distribution of an allele frequency comparison between the osteosarcoma-prone racing greyhounds and AKC greyhounds, which rarely get osteosarcoma. SNPs in the extreme tail (p<1×10-9) are highly differentiated between the two populations and are candidate germ-line osteosarcoma risk variants.

FIG. 7 is a diagram showing highly significant overlap in the set of genes altered in canine osteosarcoma tumors and two human osteosarcoma cell lines.

FIG. 8 is a diagram showing the PDGFRB pathway genes implicated in canine osteosarcoma.

FIG. 9 is a quantile-quantile plot for the Leonberger study.

FIG. 10 is a graph showing significant SNPs identified for the Leonberger study across the genome. The approximate boundaries of each chromosome on the X axis are indicated by vertical black lines.

FIG. 11 is a graph showing clustering of significant SNPs and minor allele frequency (MAF) across a region of chromosome 11 from about 37 Mb to about 44 Mb.

FIG. 12 is a graph showing clustering of significant SNPs and MAF across a region of chromosome 24 from about 25 Mb to about 35 Mb.

FIG. 13 is a graph showing clustering of significant SNPs and MAF across a region of chromosome 35 from about 9 Mb to about 14 Mb.

### DETAILED DESCRIPTION OF INVENTION

Osteosarcomas arise from mesenchymal stem cells, metastasize readily, and have widespread genetic abnormalities. Osteosarcoma in dogs is a spontaneously occurring disease with a global tumor gene expression signature indistinguishable from tumors from human pediatric patients and, while age of onset is higher in dogs, the clinical progression is remarkably similar. Both human and canine osteosarcomas most commonly arise at the ends of the long bones of the limbs and metastasize readily, usually to the lungs.

Aspects of the invention relate to germ-line risk markers (such as single nucleotide polymorphisms (SNPs), risk haplotypes, and mutations in genes) and various methods of use and/or detection thereof. The invention is premised, in part, on the results of a case-control GWAS of 304 Greyhounds, 155 Irish Wolfhounds, and 145 Rottweilers performed to identify germ-line risk markers associated with osteosarcoma. The study is described herein. Briefly, SNPs were identified that correlate with the presence of osteosarcoma in Greyhounds, Irish Wolfhounds, and/or Rottweilers. Significant SNPs were identified on chromosomes 1, 2, 3, 5, 7, 8, 9, 11, 13, 14, 15, 16, 17, 18, 19, 21, 25, 26, 32, 35, 36, and 38. These SNPs are listed in Table 1. Additionally, risk haplotypes having chromosomal regions on chromosomes 1, 2, 3, 5, 7, 8, 9, 11, 13, 14, 15, 16, 17, 18, 19, 21, 25, 26, 32, 35, 36, and 38 were identified that significantly correlated with osteosarcoma in Greyhounds, Irish Wolfhounds, and/or Rottweilers (chr11:44392734-44414985, chr8:35433142-35454649, chr13:14549973-14645634, chr25:21831580-21921256, chr14:48831824-49203827, chr5:16071171-16152955, chr19:33963105-34145310, chr16:43665149-43737129, chr15:63767963-63800415, chr16:40883517-41081510, chr25:43476429-43528145, chr1:112977233-113081800, chr3:5162058-6465753, chr7:64631053-64703475, chr1:115582915-116790630, chr2:19212450-19542015, chr1:122033806-122051988, chr35:18326079-18345318, chr9:47647012-47668054, chr38:11252518-11739329, chr21:46231985-46363479, chr17:14465884-14482152, chr32:25136302-25156153, chr36:29637804-29663408, chr15:37986345-39974762, chr1:29405587-29914411, chr26:32374093-32428448, chr25:29658978-29767164, chr26:3529343-3550075, chr5:14720254-15466603, chr18:4266743-5854451, chr1:16768869-18150476, chr9:18896060-19633155, and chr11:44390633-44406002). These germ-line risk markers were also found to correlate with canine osteosarcoma in a study involving a second, larger sample set. Additional regions were also identified in a third, follow-on study.

Accordingly, aspects of the invention provide methods that involve detecting one or more of the identified germ-line risk markers in a subject, e.g., a canine subject, in order to (a) identify a subject at elevated risk of developing osteosarcoma, or (b) identify a subject having osteosarcoma that is as yet undiagnosed. The methods can be used for prognostic purposes and for diagnostic purposes. Identifying canine subjects having an elevated risk of developing osteosarcoma is useful in a number of applications. For example, canine subjects identified as at elevated risk may be excluded from a breeding program and/or conversely canine subjects that do not carry the germ-line risk markers may be included in a breeding program. As another example, canine subjects identified as at elevated risk may be monitored, including monitored more regularly, for the appearance of osteosarcoma and/or may be treated prophylactically (e.g., prior to the development of the tumor) or therapeutically. Canine subjects carrying one or more of the germ-line risk markers may also be used to further study the progression of osteosarcoma and optionally to study the efficacy of various treatments.

In addition, in view of the clinical and histological similarity between canine osteosarcoma with human osteosarcoma, the germ-line risk markers identified in accordance with the invention may also be risk markers and/or mediators of cancer occurrence and progression in human osteosarcoma as well. Accordingly, the invention provides diagnostic and prognostic methods for use in canine subjects, animals more generally, and human subjects, as well as animal models of human disease and treatment, as well as others.

### Elevated Risk of Developing Osteosarcoma

The germ-line risk markers of the invention can be used to identify subjects at elevated risk of developing osteosarcoma. An elevated risk means a lifetime risk of developing such a cancer that is higher than the risk of developing the same cancer in (a) a population that is unselected for the presence or absence of the germ-line risk marker (i.e., the general population) or (b) a population that does not carry the germ-line risk marker.

### Osteosarcoma and Diagnostic/Prognostic Methods

Aspects of the invention include various methods, such as prognostic and diagnostic methods, related to osteosarcoma. Osteosarcoma is an aggressive malignant neoplasm arising from primitive transformed cells of mesenchymal origin. Osteosarcoma is the most common histological form of primary bone cancer in both dogs and humans. Osteosarcoma typically arises from the proximal humerus, the distal radius, the distal femur, and/or the tibia. Other sites include the ribs, the mandible, the spine, and the pelvis. In some instances, osteosarcoma may arise from soft-tissues (extraskeletal osteosarcoma). The tumor causes a great deal of pain, and can even lead to fracture of the affected bone. Metastasis of osteosarcoma tumors is very common and usually occurs in the lungs. It is to be understood that the invention provides methods for detecting germ-line risk markers regardless of the location of the osteosarcoma.

Currently available methods for diagnosis of osteosarcoma include X-ray, CT scan, PET scan, bone scan, MRI and bone biopsy. A bone biopsy may be, e.g., a needle biopsy or an open biopsy. Such methods for diagnosis may be used alone or in combination and may also be used to stage the cancer.

Osteosarcoma can be staged using, for example, the TNM system. This system uses three different codes to describe the size and location of the tumor, whether it has spread to the lymph nodes around the tumor, and whether it can be found in other parts of the body.

In the TNM system, “T” plus a letter or number (0 to 4) is used to describe the size and location of the tumor. The tumor stages for osteosarcoma are in the following table.

| | |
|---|---|
| T0 | There is no evidence of a primary tumor. |
| T1 | The tumor is 8 cm (about 3 inches) or smaller. |
| T2 | The tumor is larger than 8 cm. |
| T3 | The tumor has spread to another site or sites on the same bone. |

The “N” in the TNM system stands for node and is used to describe whether cancer has spread to regional lymph nodes. Lymph node stages are in the following table.

| | |
|---|---|
| N0 | The cancer has not spread into the regional lymph nodes. |
| N1 | The cancer has spread to regional lymph nodes. |

The “M” in the TNM system is used for cancer that has spread, or metastasized, to other parts of the body. The stages for metastatic osteosarcoma are in the following table.

| | |
|---|---|
| M0 | The cancer has not spread to the lymph nodes or other parts |
| | of the body. |
| M1 | The cancer has spread to other parts of the body. |
| M1a | The cancer has spread to the lung. |
| M1b | The cancer has spread to distant parts of the body, other |
| | than the lung. |

Tumor Grade

The TNM system also incorporates the tumor grade. The grade is generally determined by looking at cancer cells under a microscope. Tumor grades are in the following table.

| | |
|---|---|
| G1 | Low grade, cells are well differentiated |
| G2 | Low grade, cells are moderately differentiated |
| G3 | High grade, cells are poorly differentiated |
| G4 | High grade, cells are not differentiated. The cells do not look |
| | like any normal looking cells. |

Stages I to IV

After the T, N, and M categories of the osteosarcoma have been identified, this information can be combined with the tumor grade to assign a stage (I to IV) to the osteosarcoma. Stages are in the following table.

| | |
|---|---|
| Stage IA | The cancer is found only in the bone, is smaller than |
| | 8 cm, and is low grade (T1, N0, M0, G1-G2). |
| Stage IB | The cancer is found only in the bone, is larger than |
| | 8 cm, and is low grade (T2, N0, M0, G1-G2). |
| Stage IIA | The cancer is found only in the bone, is smaller than |
| | 8 cm, and is high grade (T1, N0, M0, G3-G4). |
| Stage IIB | The cancer is found only in the bone, is larger than |
| | 8 cm, and is high grade (T2, N0, M0, G3-G4). |
| Stage III | The cancer is found only in the bone but has spread to |
| | other places on the bone (T3, N0, M0, any G). |
| Stage IVA | Stage IVA - The cancer has spread to the lung (any T, |
| | N0, M1a, any G). |
| Stage IVB | The cancer has spread to lymph nodes and other parts of |
| | the body, or the cancer has spread to distant parts of |
| | the body other than the lung (Any T, N1, any M; or any T, |
| | any N, M1b, any G). |

Another staging system used is the Musculoskeletal Tumor Society (MSTS) staging system which was developed by Enneking at the University of Florida. The MSTS staging system characterizes nonmetastatic malignant bone tumors by grade (low-grade [stage I] versus high-grade [stage II]) and further subdivides these stages according to the local anatomic extent (intracompartmental [A] versus extracompartmental [B]). For bone tumors, the compartmental status is determined by whether the tumor extends through the cortex of the involved bone. The majority of high grade osteosarcoma are extracompartmental. Subjects with distant metastases are categorized as stage III.

Thus, in some embodiments, the prognostic or diagnostic methods of the invention may further comprise performing a diagnostic assay known in the art for identification and staging of osteosarcoma (e.g., x-ray, CT scan, PET scan, bone scan, MRI and/or bone biopsy).

Germ-Line Risk Markers

Aspects of the invention relate to germ-line risk markers and use and detection thereof in various methods. In general terms, a germ-line marker is a mutation in the genome of a subject that can be passed on to the offspring of the subject. Germ-line markers may or may not be risk markers. Germ-line markers are generally found in the majority, if not all, of the cells in a subject. Germ-line markers are generally inherited from one or both parents of the subject (i.e., were present in the germ cells of one or both parents). Germ-line markers as used herein also include de novo germ-line mutations, which are spontaneous mutations that occur at single-cell stage level during development. This is distinct from a somatic marker, which is a mutation in the genome of a subject that occurs after the single-cell stage during development. Somatic mutations are considered to be spontaneous mutations. Somatic mutations generally originate in a single cell or subset of cells in the subject.

A germ-line risk marker as described herein includes a SNP, a risk haplotype, or a mutation in a gene. Further discussion of each type of germ-line risk marker is provided herein. It is to be understood that a germ-line risk marker may also indicate or predict the presence of a somatic mutation in a genomic location in close proximity to the germ-line risk marker, as germ-line risk marks may correlate with a higher risk of secondary somatic mutations.

As used herein, a mutation is one or more changes in the nucleotide sequence of the genome of the subject. The terms mutation, alteration, variation, and polymorphism are used interchangeably herein. As used herein, mutations include, but are not limited to, point mutations, insertions, deletions, rearrangements, inversions and duplications. Mutations also include, but are not limited to, silent mutations, missense mutations, and nonsense mutations.

### Single Nucleotide Polymorphisms (SNPs)

In some embodiments, a germ-line risk marker is a single nucleotide polymorphism (SNP). A SNP is a mutation that occurs at a single nucleotide location on a chromosome. The nucleotide located at that position may differ between individuals in a population and/or paired chromosomes in an individual. In some embodiments, a germ-line risk marker is a SNP selected from Table 1. In some embodiments, a germ-line risk marker is a SNP selected from Table 1 or Table 5. Table 1 provides the risk nucleotide identity for each SNP (see “allele” column). The risk nucleotide is the nucleotide identity that is associated with elevated risk of developing osteosarcoma or having an undiagnosed osteosarcoma. The position (i.e., the chromosome coordinates) and SNP ID for each SNP in Table 1 are based on the CanFam 2.0 genome assembly (see, e.g., Lindblad-Toh K, Wade C M, Mikkelsen T S, Karlsson E K, Jaffe D B, Kamal M, Clamp M, Chang J L, Kulbokas E J 3rd, Zody M C, et al.: Genome sequence, comparative analysis and haplotype structure of the domestic dog. Nature 2005, 438:803-819). The first base pair in each chromosome is labeled 0 and the position of the SNP is then the number of base pairs from the first base pair (for example, the SNP on chromosome 11 at position 44405676 is located 44405676 base pairs from the first base pair of chromosome 11).

TABLE 1
List of SNPs associated with elevated risk of osteosarcoma

| | | | | | RE- |
|---|---|---|---|---|---|
| SNP | CHR | POSITION | ALLELE | BREED | GION |
| BICF2P133066 | 11 | 44405676 | A | GREY | 1 |
| BICF2P1421479 | 8 | 35448126 | C | GREY | 2 |
| BICF2S23118341 | 13 | 14588716 | T | GREY | 3 |
| BICF2G630607426 | 13 | 14615683 | T | GREY | 3 |
| BICF2G630607427 | 13 | 14616577 | G | GREY | 3 |
| BICF2G630607427 | 13 | 14616577 | A | GREY | 3 |
| BICF2G630607436 | 13 | 14628625 | T | GREY | 3 |
| BICF2G630607436 | 13 | 14628625 | A | GREY | 3 |
| BICF2P765580 | 25 | 21846080 | A | GREY | 4 |
| BICF2P811222 | 25 | 21856115 | T | GREY | 4 |
| BICF2P811227 | 25 | 21857586 | G | GREY | 4 |
| BICF2P1340261 | 25 | 21870333 | G | GREY | 4 |
| BICF2P1340261 | 25 | 21870333 | A | GREY | 4 |
| BICF2S23541806 | 25 | 21873701 | T | GREY | 4 |
| BICF2P729771 | 25 | 21894924 | T | GREY | 4 |
| BICF2S23522137 | 25 | 21898097 | C | GREY | 4 |
| BICF2S23325120 | 25 | 21912859 | T | GREY | 4 |
| BICF2S23325120 | 25 | 21912859 | A | GREY | 4 |
| BICF2P740200 | 25 | 21914402 | G | GREY | 4 |
| BICF2S23117654 | 14 | 48841157 | C | GREY | 5 |
| BICF2P412246 | 14 | 48863885 | T | GREY | 5 |
| BICF2P257681 | 14 | 48907365 | G | GREY | 5 |
| BICF2S23156412 | 14 | 48912477 | A | GREY | 5 |
| BICF2S23246280 | 14 | 48969098 | T | GREY | 5 |
| BICF2S23245388 | 14 | 49046791 | A | GREY | 5 |
| BICF2S23715556 | 14 | 49085738 | A | GREY | 5 |
| BICF2P66597 | 14 | 49193217 | G | GREY | 5 |
| BICF2P1194727 | 5 | 16085937 | G | GREY | 6 |
| BICF2P381887 | 5 | 16093715 | T | GREY | 6 |
| BICF2P381179 | 5 | 16108115 | G | GREY | 6 |
| BICF2P968528 | 5 | 16114280 | A | GREY | 6 |
| BICF2P1248827 | 5 | 16145308 | C | GREY | 6 |
| BICF2G63051679 | 19 | 33978816 | A | GREY | 7 |
| BICF2G63051718 | 19 | 33999957 | T | GREY | 7 |
| BICF2P1270504 | 19 | 34023257 | G | GREY | 7 |
| BICF2G63051809 | 19 | 34134931 | T | GREY | 7 |
| BICF2G630813090 | 16 | 43669044 | C | GREY | 8 |
| BICF2G630813102 | 16 | 43676315 | T | GREY | 8 |
| BICF2G630813102 | 16 | 43676315 | T | GREY | 8 |
| BICF2P1152672 | 16 | 43712139 | G | GREY | 8 |
| BICF2P1109206 | 16 | 43725055 | A | GREY | 8 |
| BICF2G630418573 | 15 | 63780452 | A | GREY | 9 |
| TIGRP2P215623 | 16 | 40896559 | C | GREY | 10 |
| BICF2P357340 | 16 | 41073136 | A | GREY | 10 |
| TIGRP2P331221 | 25 | 43485109 | G | GREY | 11 |
| TIGRP2P331223 | 25 | 43495780 | G | GREY | 11 |
| BICF2S23445991 | 25 | 43511232 | T | GREY | 11 |
| TIGRP2P331236 | 25 | 43519306 | C | GREY | 11 |
| BICF2P229090 | 1 | 112990217 | A | GREY | 12 |
| BICF2S23516022 | 1 | 112990983 | C | GREY | 12 |
| BICF2S22922837 | 1 | 113043126 | G | GREY | 12 |
| BICF2P774726 | 3 | 5170412 | A | GREY | 13 |
| BICF2P879101 | 3 | 5211310 | C | GREY | 13 |
| BICF2P379530 | 3 | 5233667 | A | GREY | 13 |
| BICF2P475099 | 3 | 5245090 | C | GREY | 13 |
| TIGRP2P45104 | 3 | 5260931 | A | GREY | 13 |
| BICF2P150216 | 3 | 5299580 | G | GREY | 13 |
| BICF2P529456 | 3 | 5328933 | C | GREY | 13 |
| TIGRP2P45112 | 3 | 5338620 | A | GREY | 13 |
| BICF2P194703 | 3 | 5371137 | A | GREY | 13 |
| BICF2P209319 | 3 | 5384473 | T | GREY | 13 |
| BICF2P22627 | 3 | 5401853 | G | GREY | 13 |
| TIGRP2P45129 | 3 | 5410018 | C | GREY | 13 |
| BICF2P1038896 | 3 | 5426478 | G | GREY | 13 |
| BICF2S2337294 | 3 | 5436506 | C | GREY | 13 |
| BICF2P785126 | 3 | 5441183 | G | GREY | 13 |
| BICF2P1209289 | 3 | 5456793 | C | GREY | 13 |
| TIGRP2P45140 | 3 | 5471514 | A | GREY | 13 |
| TIGRP2P45143 | 3 | 5475615 | A | GREY | 13 |
| TIGRP2P45151 | 3 | 5487857 | T | GREY | 13 |
| TIGRP2P45157 | 3 | 5508995 | A | GREY | 13 |
| BICF2P112727 | 3 | 5521110 | G | GREY | 13 |
| BICF2P148547 | 3 | 5534827 | G | GREY | 13 |
| TIGRP2P45171 | 3 | 5564882 | T | GREY | 13 |
| BICF2P761881 | 3 | 5573179 | G | GREY | 13 |
| BICF2P363714 | 3 | 5581700 | C | GREY | 13 |
| BICF2P1010972 | 3 | 5592544 | A | GREY | 13 |
| TIGRP2P45176 | 3 | 5638043 | G | GREY | 13 |
| TIGRP2P45178 | 3 | 5647032 | C | GREY | 13 |
| BICF2P1026069 | 3 | 5700685 | G | GREY | 13 |
| BICF2P327737 | 3 | 5709075 | C | GREY | 13 |
| TIGRP2P45197 | 3 | 5719522 | A | GREY | 13 |
| BICF2S22960989 | 3 | 5736343 | G | GREY | 13 |
| TIGRP2P45218 | 3 | 5742823 | G | GREY | 13 |
| BICF2P1190297 | 3 | 5752510 | C | GREY | 13 |
| BICF2S2328630 | 3 | 5770706 | G | GREY | 13 |
| BICF2P840628 | 3 | 5806192 | C | GREY | 13 |
| TIGRP2P45242 | 3 | 5834019 | T | GREY | 13 |
| TIGRP2P45246 | 3 | 5864745 | G | GREY | 13 |
| TIGRP2P45249 | 3 | 5876747 | A | GREY | 13 |
| BICF2P721123 | 3 | 5899710 | A | GREY | 13 |
| TIGRP2P45259 | 3 | 5942967 | C | GREY | 13 |
| TIGRP2P45264 | 3 | 5960454 | G | GREY | 13 |
| BICF2P1018229 | 3 | 5970041 | G | GREY | 13 |
| TIGRP2P45267 | 3 | 5988284 | G | GREY | 13 |
| BICF2P569426 | 3 | 6000198 | A | GREY | 13 |
| BICF2P238213 | 3 | 6009648 | A | GREY | 13 |
| TIGRP2P45276 | 3 | 6048543 | G | GREY | 13 |
| BICF2P1023047 | 3 | 6148587 | T | GREY | 13 |
| BICF2P133199 | 3 | 6216098 | G | GREY | 13 |
| BICF2P556026 | 3 | 6241715 | A | GREY | 13 |
| BICF2P510160 | 3 | 6257542 | C | GREY | 13 |
| TIGRP2P45301 | 3 | 6272593 | C | GREY | 13 |
| BICF2P412371 | 3 | 6280200 | G | GREY | 13 |
| BICF2P152439 | 3 | 6305511 | T | GREY | 13 |
| TIGRP2P45306 | 3 | 6319525 | G | GREY | 13 |
| BICF2P1458832 | 3 | 6417921 | A | GREY | 13 |
| BICF2P158838 | 3 | 6433958 | T | GREY | 13 |
| BICF2S23717124 | 3 | 6453902 | G | GREY | 13 |
| BICF2G630564029 | 7 | 64646068 | T | GREY | 14 |
| BICF2G630564047 | 7 | 64664054 | T | GREY | 14 |
| BICF2P1090686 | 7 | 64672328 | C | GREY | 14 |
| BICF2G630564076 | 7 | 64690186 | T | GREY | 14 |
| BICF2S23535303 | 5 | 14740098 | C | IWH | 1 |
| BICF2S23520119 | 5 | 14759439 | T | IWH | 1 |
| G326F32S322 | 5 | 14786078 | C | IWH | 1 |
| BICF2S23238674 | 5 | 14999278 | T | IWH | 1 |
| BICF2P645758 | 5 | 15006237 | T | IWH | 1 |
| BICF2P189890 | 5 | 15009903 | G | IWH | 1 |
| BICF2P819174 | 5 | 15062440 | T | IWH | 1 |
| BICF2P162666 | 5 | 15104598 | G | IWH | 1 |
| BICF2P1366853 | 5 | 15123506 | C | IWH | 1 |
| BICF2P775251 | 5 | 15254166 | C | IWH | 1 |
| BICF2S23746532 | 5 | 15264066 | A | IWH | 1 |
| BICF2P1162557 | 5 | 15333462 | G | IWH | 1 |
| BICF2S23538747 | 5 | 15391207 | G | IWH | 1 |
| BICF2S23538670 | 5 | 15395179 | C | IWH | 1 |
| BICF2S23218055 | 5 | 15433708 | G | IWH | 1 |
| BICF2P680751 | 5 | 15448667 | C | IWH | 1 |
| BICF2S23510137 | 18 | 4281291 | T | IWH | 2 |
| BICF2P849639 | 18 | 4315994 | T | IWH | 2 |
| BICF2S22945333 | 18 | 4688275 | T | IWH | 2 |
| BICF2S2298851 | 18 | 4705260 | A | IWH | 2 |
| TIGRP2P238123 | 18 | 4824662 | A | IWH | 2 |
| TIGRP2P238132 | 18 | 4929731 | T | IWH | 2 |
| BICF2P1466354 | 18 | 4937944 | C | IWH | 2 |
| BICF2P440326 | 18 | 4951290 | C | IWH | 2 |
| BICF2P874005 | 18 | 4959653 | C | IWH | 2 |
| BICF2P928021 | 18 | 4971847 | G | IWH | 2 |
| BICF2P1182592 | 18 | 4980153 | C | IWH | 2 |
| BICF2P1378069 | 18 | 4994445 | T | IWH | 2 |
| TIGRP2P238162 | 18 | 5005071 | G | IWH | 2 |
| TIGRP2P253880 | 18 | 5212336 | T | IWH | 2 |
| BICF2P461252 | 18 | 5283915 | T | IWH | 2 |
| BICF2P879737 | 18 | 5291054 | G | IWH | 2 |
| BICF2P163146 | 18 | 5296804 | T | IWH | 2 |
| BICF2S23259485 | 18 | 5338019 | A | IWH | 2 |
| TIGRP2P253975 | 18 | 5362366 | C | IWH | 2 |
| BICF2S23760612 | 18 | 5374587 | A | IWH | 2 |
| TIGRP2P254013 | 18 | 5396556 | C | IWH | 2 |
| TIGRP2P254028 | 18 | 5406832 | A | IWH | 2 |
| BICF2S23750273 | 18 | 5422762 | A | IWH | 2 |
| BICF2P228579 | 18 | 5430590 | T | IWH | 2 |
| TIGRP2P254054 | 18 | 5449914 | C | IWH | 2 |
| BICF2P531896 | 18 | 5457566 | G | IWH | 2 |
| TIGRP2P254060 | 18 | 5467639 | A | IWH | 2 |
| BICF2P766570 | 18 | 5479664 | G | IWH | 2 |
| BICF2P1014267 | 18 | 5493249 | A | IWH | 2 |
| BICF2P1006929 | 18 | 5512082 | G | IWH | 2 |
| BICF2P1299781 | 18 | 5522052 | T | IWH | 2 |
| BICF2P672676 | 18 | 5543603 | G | IWH | 2 |
| BICF2S23761559 | 18 | 5562106 | A | IWH | 2 |
| BICF2P15617 | 18 | 5572732 | C | IWH | 2 |
| BICF2P439160 | 18 | 5587296 | A | IWH | 2 |
| TIGRP2P254095 | 18 | 5594969 | T | IWH | 2 |
| TIGRP2P254109 | 18 | 5605791 | C | IWH | 2 |
| BICF2P477812 | 18 | 5619906 | G | IWH | 2 |
| BICF2P1238318 | 18 | 5629193 | T | IWH | 2 |
| BICF2P1354921 | 18 | 5652736 | A | IWH | 2 |
| BICF2S23741435 | 18 | 5661990 | A | IWH | 2 |
| BICF2P37118 | 18 | 5694792 | C | IWH | 2 |
| TIGRP2P254175 | 18 | 5713966 | C | IWH | 2 |
| BICF2P1123483 | 18 | 5726554 | C | IWH | 2 |
| TIGRP2P254184 | 18 | 5728091 | T | IWH | 2 |
| BICF2P825842 | 18 | 5765212 | T | IWH | 2 |
| BICF2P243632 | 18 | 5783766 | A | IWH | 2 |
| BICF2P1139856 | 18 | 5794059 | A | IWH | 2 |
| BICF2P1376844 | 18 | 5796973 | C | IWH | 2 |
| TIGRP2P254212 | 18 | 5826824 | C | IWH | 2 |
| TIGRP2P254216 | 18 | 5834540 | G | IWH | 2 |
| TIGRP2P254223 | 18 | 5845556 | G | IWH | 2 |
| BICF2P426201 | 1 | 16782692 | C | IWH | 3 |
| BICF2S2436535 | 1 | 16851821 | C | IWH | 3 |
| BICF2P460868 | 1 | 16997009 | A | IWH | 3 |
| BICF2P252171 | 1 | 17009485 | A | IWH | 3 |
| TIGRP2P15921 | 1 | 17060348 | G | IWH | 3 |
| TIGRP2P15926 | 1 | 17068084 | A | IWH | 3 |
| BICF2S22943775 | 1 | 17098395 | C | IWH | 3 |
| BICF2P300536 | 1 | 17109675 | G | IWH | 3 |
| BICF2P69061 | 1 | 17130042 | T | IWH | 3 |
| BICF2P931019 | 1 | 17133605 | A | IWH | 3 |
| BICF2P172990 | 1 | 17142397 | C | IWH | 3 |
| TIGRP2P15939 | 1 | 17163604 | C | IWH | 3 |
| TIGRP2P15940 | 1 | 17180308 | G | IWH | 3 |
| BICF2P1166345 | 1 | 17182762 | G | IWH | 3 |
| TIGRP2P15943 | 1 | 17196849 | G | IWH | 3 |
| BICF2S2334324 | 1 | 17226603 | C | IWH | 3 |
| BICF2S23638404 | 1 | 17342812 | T | IWH | 3 |
| BICF2P510074 | 1 | 17400867 | A | IWH | 3 |
| TIGRP2P16004 | 1 | 17493415 | T | IWH | 3 |
| TIGRP2P16009 | 1 | 17497796 | G | IWH | 3 |
| BICF2P1225386 | 1 | 17742179 | C | IWH | 3 |
| BICF2P976808 | 1 | 17746147 | C | IWH | 3 |
| BICF2P572866 | 1 | 17780758 | T | IWH | 3 |
| BICF2P51884 | 1 | 17795209 | C | IWH | 3 |
| TIGRP2P16037 | 1 | 17806466 | C | IWH | 3 |
| BICF2P230951 | 1 | 17814464 | T | IWH | 3 |
| BICF2P674288 | 1 | 17830618 | A | IWH | 3 |
| BICF2G630711956 | 1 | 18126292 | G | IWH | 3 |
| BICF2P718275 | 9 | 18911590 | A | IWH | 4 |
| BICF2S23223892 | 9 | 18996332 | A | IWH | 4 |
| BICF2P328758 | 9 | 18998670 | T | IWH | 4 |
| BICF2P158760 | 9 | 19014494 | C | IWH | 4 |
| BICF2S23055739 | 9 | 19037490 | T | IWH | 4 |
| BICF2S23516922 | 9 | 19084362 | G | IWH | 4 |
| BICF2S2438924 | 9 | 19096254 | T | IWH | 4 |
| BICF2S2294380 | 9 | 19121052 | G | IWH | 4 |
| BICF2S2294380 | 9 | 19121052 | G | IWH | 4 |
| BICF2S2439997 | 9 | 19128539 | T | IWH | 4 |
| BICF2S2439997 | 9 | 19128539 | T | IWH | 4 |
| BICF2P452889 | 9 | 19160959 | T | IWH | 4 |
| BICF2P364366 | 9 | 19183929 | C | IWH | 4 |
| BICF2P47724 | 9 | 19194063 | C | IWH | 4 |
| BICF2P438701 | 9 | 19281859 | A | IWH | 4 |
| BICF2P438701 | 9 | 19281859 | A | IWH | 4 |
| BICF2P589810 | 9 | 19289821 | C | IWH | 4 |
| BICF2P589810 | 9 | 19289821 | C | IWH | 4 |
| BICF2P394578 | 9 | 19312506 | T | IWH | 4 |
| TIGRP2P122861 | 9 | 19338752 | T | IWH | 4 |
| TIGRP2P122861 | 9 | 19338752 | C | IWH | 4 |
| BICF2P1079087 | 9 | 19349148 | A | IWH | 4 |
| BICF2P579696 | 9 | 19381199 | T | IWH | 4 |
| BICF2P497312 | 9 | 19396139 | C | IWH | 4 |
| BICF2P1230700 | 9 | 19418665 | T | IWH | 4 |
| TIGRP2P122831 | 9 | 19498086 | C | IWH | 4 |
| BICF2P422703 | 9 | 19601839 | G | IWH | 4 |
| BICF2P1125643 | 9 | 19623231 | C | IWH | 4 |
| BICF2P1125643 | 9 | 19623231 | C | IWH | 4 |
| BICF2P1125643 | 9 | 19623231 | T | IWH | 4 |
| BICF2S2308696 | 1 | 115597438 | A | ROTT | 1 |
| BICF2P508906 | 1 | 115630525 | C | ROTT | 1 |
| BICF2P508905 | 1 | 115631080 | T | ROTT | 1 |
| BICF2S23216058 | 1 | 115636142 | C | ROTT | 1 |
| BICF2S23216058 | 1 | 115636142 | T | ROTT | 1 |
| BICF2P266591 | 1 | 115646651 | A | ROTT | 1 |
| BICF2P1332375 | 1 | 115759072 | A | ROTT | 1 |
| BICF2S23231062 | 1 | 115892937 | C | ROTT | 1 |
| BICF2S22945043 | 1 | 115944412 | G | ROTT | 1 |
| BICF2P326880 | 1 | 115961693 | T | ROTT | 1 |
| BICF2P893664 | 1 | 116008326 | T | ROTT | 1 |
| BICF2P1420547 | 1 | 116028956 | A | ROTT | 1 |
| BICF2P698281 | 1 | 116217014 | A | ROTT | 1 |
| BICF2S22919383 | 1 | 116340675 | G | ROTT | 1 |
| BICF2S22947803 | 1 | 116361619 | A | ROTT | 1 |
| BICF2S22947803 | 1 | 116361619 | A | ROTT | 1 |
| BICF2S22959094 | 1 | 116371940 | C | ROTT | 1 |
| BICF2S23228287 | 1 | 116399288 | A | ROTT | 1 |
| BICF2S23036972 | 1 | 116403385 | A | ROTT | 1 |
| BICF2P51623 | 1 | 116415896 | C | ROTT | 1 |
| BICF2P1346510 | 1 | 116479292 | T | ROTT | 1 |
| BICF2P1323908 | 1 | 116492092 | G | ROTT | 1 |
| BICF2P1137984 | 1 | 116508040 | C | ROTT | 1 |
| BICF2P1115364 | 1 | 116524913 | G | ROTT | 1 |
| BICF2P58266 | 1 | 116596060 | G | ROTT | 1 |
| BICF2P627162 | 1 | 116619074 | C | ROTT | 1 |
| BICF2P1422910 | 1 | 116627544 | G | ROTT | 1 |
| BICF2P162782 | 1 | 116637538 | A | ROTT | 1 |
| BICF2P162782 | 1 | 116637538 | G | ROTT | 1 |
| BICF2P1342901 | 1 | 116649825 | T | ROTT | 1 |
| BICF2P868731 | 1 | 116659732 | C | ROTT | 1 |
| BICF2P768889 | 1 | 116667958 | A | ROTT | 1 |
| BICF2P1052528 | 1 | 116688554 | T | ROTT | 1 |
| BICF2P408119 | 1 | 116694244 | A | ROTT | 1 |
| BICF2P1468011 | 1 | 116704630 | A | ROTT | 1 |
| BICF2P219326 | 1 | 116735064 | A | ROTT | 1 |
| BICF2P1462759 | 1 | 116744917 | C | ROTT | 1 |
| BICF2P307386 | 1 | 116753371 | T | ROTT | 1 |
| BICF2P1010170 | 1 | 116761963 | C | ROTT | 1 |
| BICF2S23038485 | 2 | 19229091 | C | ROTT | 2 |
| BICF2G630672865 | 2 | 19244386 | A | ROTT | 2 |
| BICF2G630672813 | 2 | 19288113 | G | ROTT | 2 |
| BICF2P1369145 | 2 | 19360520 | C | ROTT | 2 |
| BICF2G630672770 | 2 | 19383308 | A | ROTT | 2 |
| BICF2P81989 | 2 | 19402118 | C | ROTT | 2 |
| BICF2P916235 | 2 | 19426763 | G | ROTT | 2 |
| BICF2G630672753 | 2 | 19462778 | A | ROTT | 2 |
| BICF2P1177075 | 2 | 19483009 | C | ROTT | 2 |
| BICF2P411325 | 2 | 19515571 | C | ROTT | 2 |
| BICF2P1210630 | 1 | 122048812 | C | ROTT | 3 |
| TIGRP2P407733 | 35 | 18338700 | A | ROTT | 4 |
| BICF2P341331 | 9 | 47659782 | A | ROTT | 5 |
| BICF2P318350 | 38 | 11259655 | C | ROTT | 6 |
| BICF2S2335735 | 38 | 11303710 | T | ROTT | 6 |
| BICF2P1003572 | 38 | 11361700 | A | ROTT | 6 |
| BICF2P1104551 | 38 | 11400039 | A | ROTT | 6 |
| BICF2S23550277 | 38 | 11455402 | G | ROTT | 6 |
| BICF2P870378 | 38 | 11480634 | A | ROTT | 6 |
| BICF2P866460 | 38 | 11489608 | T | ROTT | 6 |
| BICF2P1303772 | 38 | 11506377 | G | ROTT | 6 |
| BICF2S23738710 | 38 | 11512589 | C | ROTT | 6 |
| BICF2P344455 | 38 | 11526769 | C | ROTT | 6 |
| BICF2P825177 | 38 | 11541386 | A | ROTT | 6 |
| BICF2S23324500 | 38 | 11589162 | G | ROTT | 6 |
| BICF2S23544574 | 38 | 11618856 | C | ROTT | 6 |
| BICF2P119783 | 38 | 11641925 | A | ROTT | 6 |
| BICF2S23758510 | 38 | 11673310 | C | ROTT | 6 |
| BICF2S23724888 | 38 | 11684867 | C | ROTT | 6 |
| BICF2P1129874 | 38 | 11714169 | T | ROTT | 6 |
| BICF2P254147 | 21 | 46251007 | C | ROTT | 7 |
| BICF2S2376247 | 21 | 46254772 | G | ROTT | 7 |
| TIGRP2P286750 | 21 | 46283811 | C | ROTT | 7 |
| BICF2P171066 | 21 | 46308873 | A | ROTT | 7 |
| TIGRP2P286767 | 21 | 46334269 | T | ROTT | 7 |
| TIGRP2P286795 | 21 | 46349601 | G | ROTT | 7 |
| BICF2S23533459 | 17 | 14472761 | C | ROTT | 8 |
| BICF2G630590368 | 32 | 25147661 | A | ROTT | 9 |
| BICF2P92014 | 36 | 29651125 | A | ROTT | 10 |
| BICF2S23339954 | 15 | 37998132 | T | ROTT | 11 |
| TIGRP2P199934 | 15 | 38019697 | A | ROTT | 11 |
| TIGRP2P199940 | 15 | 38033078 | T | ROTT | 11 |
| TIGRP2P199942 | 15 | 38042634 | C | ROTT | 11 |
| BICF2S23157944 | 15 | 38264190 | T | ROTT | 11 |
| BICF2S22922723 | 15 | 38304593 | T | ROTT | 11 |
| BICF2P752717 | 15 | 38499624 | A | ROTT | 11 |
| BICF2P382393 | 15 | 38512016 | T | ROTT | 11 |
| BICF2P334525 | 15 | 38516288 | T | ROTT | 11 |
| BICF2P187948 | 15 | 38531916 | T | ROTT | 11 |
| BICF2P360394 | 15 | 38551281 | G | ROTT | 11 |
| BICF2S23032337 | 15 | 38597342 | A | ROTT | 11 |
| BICF2P881582 | 15 | 38619239 | G | ROTT | 11 |
| BICF2P178377 | 15 | 38646852 | T | ROTT | 11 |
| BICF2S22912603 | 15 | 38674144 | A | ROTT | 11 |
| BICF2P1031206 | 15 | 38746955 | C | ROTT | 11 |
| TIGRP2P200003 | 15 | 38767028 | C | ROTT | 11 |
| BICF2S23018355 | 15 | 38769763 | A | ROTT | 11 |
| BICF2P1020099 | 15 | 38800344 | C | ROTT | 11 |
| TIGRP2P200032 | 15 | 38904027 | C | ROTT | 11 |
| TIGRP2P200033 | 15 | 38909750 | G | ROTT | 11 |
| BICF2P217898 | 15 | 38919888 | A | ROTT | 11 |
| TIGRP2P200071 | 15 | 38987072 | T | ROTT | 11 |
| TIGRP2P200088 | 15 | 39001982 | T | ROTT | 11 |
| BICF2P1083029 | 15 | 39024671 | A | ROTT | 11 |
| BICF2S23034677 | 15 | 39042745 | T | ROTT | 11 |
| BICF2S2375443 | 15 | 39161851 | G | ROTT | 11 |
| BICF2P1149468 | 15 | 39169562 | G | ROTT | 11 |
| BICF2P742493 | 15 | 39188468 | T | ROTT | 11 |
| BICF2P1309224 | 15 | 39210513 | G | ROTT | 11 |
| BICF2S23720644 | 15 | 39215533 | T | ROTT | 11 |
| BICF2S23113349 | 15 | 39241402 | G | ROTT | 11 |
| TIGRP2P200144 | 15 | 39271970 | T | ROTT | 11 |
| BICF2P1278650 | 15 | 39273742 | T | ROTT | 11 |
| BICF2P1093819 | 15 | 39323300 | G | ROTT | 11 |
| TIGRP2P200166 | 15 | 39346724 | G | ROTT | 11 |
| BICF2P459854 | 15 | 39353529 | T | ROTT | 11 |
| BICF2P587000 | 15 | 39367943 | T | ROTT | 11 |
| BICF2P778010 | 15 | 39492981 | T | ROTT | 11 |
| BICF2P325024 | 15 | 39516234 | T | ROTT | 11 |
| BICF2P472555 | 15 | 39553988 | T | ROTT | 11 |
| BICF2P307089 | 15 | 39561516 | A | ROTT | 11 |
| BICF2P307089 | 15 | 39561516 | G | ROTT | 11 |
| TIGRP2P200235 | 15 | 39605173 | C | ROTT | 11 |
| TIGRP2P200235 | 15 | 39605173 | A | ROTT | 11 |
| BICF2S23344310 | 15 | 39622801 | A | ROTT | 11 |
| BICF2S23344310 | 15 | 39622801 | G | ROTT | 11 |
| BICF2P1143365 | 15 | 39634571 | G | ROTT | 11 |
| BICF2P1162997 | 15 | 39652018 | A | ROTT | 11 |
| TIGRP2P200259 | 15 | 39690799 | G | ROTT | 11 |
| BICF2P1197030 | 15 | 39698607 | C | ROTT | 11 |
| TIGRP2P200265 | 15 | 39712997 | G | ROTT | 11 |
| BICF2P244321 | 15 | 39760788 | C | ROTT | 11 |
| BICF2P244321 | 15 | 39760788 | A | ROTT | 11 |
| BICF2P654662 | 15 | 39790921 | A | ROTT | 11 |
| BICF2P690478 | 15 | 39858245 | A | ROTT | 11 |
| TIGRP2P200321 | 15 | 39903893 | G | ROTT | 11 |
| BICF2P647591 | 15 | 39940786 | T | ROTT | 11 |
| BICF2S23719922 | 15 | 39962039 | T | ROTT | 11 |
| BICF2P1206600 | 15 | 39964571 | C | ROTT | 11 |
| BICF2P848519 | 1 | 29420845 | A | ROTT | 12 |
| BICF2P1096901 | 1 | 29442608 | T | ROTT | 12 |
| BICF2P270493 | 1 | 29444774 | G | ROTT | 12 |
| BICF2P378388 | 1 | 29542070 | C | ROTT | 12 |
| BICF2P104206 | 1 | 29560184 | T | ROTT | 12 |
| BICF2S2343850 | 1 | 29668952 | A | ROTT | 12 |
| BICF2P1164085 | 1 | 29775073 | G | ROTT | 12 |
| BICF2P806301 | 1 | 29785710 | T | ROTT | 12 |
| BICF2P1433886 | 1 | 29902244 | A | ROTT | 12 |
| BICF2S23712115 | 26 | 32385934 | G | ROTT | 13 |
| BICF2S23712114 | 26 | 32386262 | T | ROTT | 13 |
| BICF2G63095567 | 25 | 29671618 | G | ROTT | 14 |
| BICF2G63095567 | 25 | 29671618 | G | ROTT | 14 |
| BICF2G63095608 | 25 | 29693487 | G | ROTT | 14 |
| BICF2G63095608 | 25 | 29693487 | G | ROTT | 14 |
| BICF2G63095630 | 25 | 29713336 | A | ROTT | 14 |
| BICF2G63095630 | 25 | 29713336 | A | ROTT | 14 |
| BICF2G63095645 | 25 | 29736474 | G | ROTT | 14 |
| BICF2G63095650 | 25 | 29758499 | G | ROTT | 14 |
| BICF2G63095650 | 25 | 29758499 | G | ROTT | 14 |
| BICF2P841536 | 26 | 3537143 | A | ROTT | 15 |

CHR = chromosome, ALLELE = risk nucleotide, BREED = breed identified in Table 4 in Examples (GREY = Greyhound, IWH = Irish Wolfhound, ROTT = Rottweiler), REGION refers to column 1 of Table 4 in Examples.

In some embodiments, the SNP may be one or more of:
i) one or more chromosome 1 SNPs,
ii) one or more chromosome 2 SNPs,
iii) one or more chromosome 3 SNPs,
iv) one or more chromosome 5 SNPs,
v) one or more chromosome 7 SNPs,
vi) one or more chromosome 8 SNPs,
vii) one or more chromosome 9 SNPs,
viii) one or more chromosome 11 SNPs,
ix) one or more chromosome 13 SNPs,
x) one or more chromosome 14 SNPs,
xi) one or more chromosome 15 SNPs,
xii) one or more chromosome 16 SNPs,
xii) one or more chromosome 17 SNPs,
xiv) one or more chromosome 18 SNPs,
xv) one or more chromosome 19 SNPs,
xvi) one or more chromosome 21 SNPs,
xvii) one or more chromosome 25 SNPs,
xvii) one or more chromosome 26 SNPs,
xix) one or more chromosome 32 SNPs,
xx) one or more chromosome 35 SNPs,
xxi) one or more chromosome 36 SNPs, and
xxii) one or more chromosome 38 SNPs, all of which are provided in Table 1.

In some embodiments, a SNP may be used in the methods described herein. In some embodiments, the method comprises:

a) analyzing genomic DNA from a canine subject for the presence of a SNP selected from:
i) one or more chromosome 1 SNPs,
ii) one or more chromosome 2 SNPs,
iii) one or more chromosome 3 SNPs,
iv) one or more chromosome 5 SNPs,
v) one or more chromosome 7 SNPs,
vi) one or more chromosome 8 SNPs,
vii) one or more chromosome 9 SNPs,
viii) one or more chromosome 11 SNPs,
ix) one or more chromosome 13 SNPs,
x) one or more chromosome 14 SNPs,
xi) one or more chromosome 15 SNPs,
xii) one or more chromosome 16 SNPs,
xii) one or more chromosome 17 SNPs,
xiv) one or more chromosome 18 SNPs,
xv) one or more chromosome 19 SNPs,
xvi) one or more chromosome 21 SNPs,
xvii) one or more chromosome 25 SNPs,
xvii) one or more chromosome 26 SNPs,
xix) one or more chromosome 32 SNPs,
xx) one or more chromosome 35 SNPs,
xxi) one or more chromosome 36 SNPs, and
xxii) one or more chromosome 38 SNPs; and

b) identifying the canine subject having one or more of the SNPs as a subject (a) at elevated risk of developing osteosarcoma or (b) having an undiagnosed osteosarcoma.

In some embodiments, the SNP is selected from BICF2P133066, BICF2P1421479, BICF2S2308696, BICF2P508906, BICF2P508905, BICF2S23216058, BICF2S23216058, BICF2P266591, BICF2P1332375, BICF2S23231062, BICF2S22945043, BICF2P326880, BICF2P893664, BICF2P1420547, BICF2P698281, BICF2S22919383, BICF2S22947803, BICF2S22947803, BICF2S22959094, BICF2S23228287, BICF2S23036972, BICF2P51623, BICF2P1346510, BICF2P1323908, BICF2P1137984, BICF2P1115364, BICF2P58266, BICF2P627162, BICF2P1422910, BICF2P162782, BICF2P162782, BICF2P1342901, BICF2P868731, BICF2P768889, BICF2P1052528, BICF2P408119, BICF2P1468011, BICF2P219326, BICF2P1462759, BICF2P307386, BICF2P1010170, BICF2S23038485, BICF2G630672865, BICF2G630672813, BICF2P1369145, BICF2G630672770, BICF2P81989, BICF2P916235, BICF2G630672753, BICF2P1177075, BICF2P411325, BICF2P1210630, TIGRP2P407733, BICF2P341331, BICF2P318350, BICF2S2335735, BICF2P1003572, BICF2P1104551, BICF2S23550277, BICF2P870378, BICF2P866460, BICF2P1303772, BICF2S23738710, BICF2P344455, BICF2P825177, BICF2S23324500, BICF2S23544574, BICF2P119783, BICF2S23758510, BICF2S23724888, BICF2P1129874, BICF2S23535303, BICF2S23520119, G326F32S322, BICF2S23238674, BICF2P645758, BICF2P189890, BICF2P819174, BICF2P162666, BICF2P1366853, BICF2P775251, BICF2S23746532, BICF2P1162557, BICF2S23538747, BICF2S23538670, BICF2S23218055, BICF2P680751, BICF2S23510137, BICF2P849639, BICF2S22945333, BICF2S2298851, TIGRP2P238123, TIGRP2P238132, BICF2P1466354, BICF2P440326, BICF2P874005, BICF2P928021, BICF2P1182592, BICF2P1378069, TIGRP2P238162, TIGRP2P253880, BICF2P461252, BICF2P879737, BICF2P163146, BICF2S23259485, TIGRP2P253975, BICF2S23760612, TIGRP2P254013, TIGRP2P254028, BICF2S23750273, BICF2P228579, TIGRP2P254054, BICF2P531896, TIGRP2P254060, BICF2P766570, BICF2P1014267, BICF2P1006929, BICF2P1299781, BICF2P672676, BICF2S23761559, BICF2P15617, BICF2P439160, TIGRP2P254095, TIGRP2P254109, BICF2P477812, BICF2P1238318, BICF2P1354921, BICF2S23741435, BICF2P37118, TIGRP2P254175, BICF2P1123483, TIGRP2P254184, BICF2P825842, BICF2P243632, BICF2P1139856, BICF2P1376844, TIGRP2P254212, TIGRP2P254216, or TIGRP2P254223.

In some embodiments, the SNP is selected from BICF2P133066, BICF2S2308696, BICF2P508906, BICF2P508905, BICF2S23216058, BICF2S23216058, BICF2P266591, BICF2P1332375, BICF2S23231062, BICF2S22945043, BICF2P326880, BICF2P893664, BICF2P1420547, BICF2P698281, BICF2S22919383, BICF2S22947803, BICF2S22947803, BICF2S22959094, BICF2S23228287, BICF2S23036972, BICF2P51623, BICF2P1346510, BICF2P1323908, BICF2P1137984, BICF2P1115364, BICF2P58266, BICF2P627162, BICF2P1422910, BICF2P162782, BICF2P162782, BICF2P1342901, BICF2P868731, BICF2P768889, BICF2P1052528, BICF2P408119, BICF2P1468011, BICF2P219326, BICF2P1462759, BICF2P307386, BICF2P1010170, BICF2P229090, BICF2S23516022, or BICF2S22922837.

In some embodiments, the SNP is BICF2P133066.

It is to be understood that any number of SNPs (e.g., 1, 2, 3, 4, 5, 6, 7, 8, 9, 10, 11, 12, 13, 14, 15, 16, 17, 18, 19, or 20 or more SNPs) may be detected and/or used to identify a subject.

### Risk Haplotypes

In some embodiments, a germ-line risk marker is a risk haplotype. A risk haplotype, as used herein, is a chromosomal region containing at least one mutation that correlates with the presence of or likelihood of developing osteosarcoma in a subject. A risk haplotype is detected or identified and/or may be defined by one or more mutations. For example, a risk haplotype may be a chromosomal region with boundaries that are defined by two or more SNPs that are in linkage disequilibrium and correlate with the presence of or likelihood of developing osteosarcoma in a subject. Such SNPs may themselves be disease-causative or may, alternatively or additionally, be indicators of other mutations (either germ-line mutations or somatic mutations) present in the chromosomal region of the risk haplotype that correlate with or cause osteosarcoma in a subject. Thus, other mutations within the risk haplotype may correlate with presence of or likelihood of developing osteosarcoma in a subject and are contemplated for use in the methods herein. Accordingly, in some embodiments, methods described herein comprise use and/or detection of a risk haplotype. In some embodiments, the risk haplotype is selected from:

a risk haplotype having chromosome coordinates chr11:44392734-44414985,

a risk haplotype having chromosome coordinates chr8:35433142-35454649,

a risk haplotype having chromosome coordinates chr13:14549973-14645634,

a risk haplotype having chromosome coordinates chr25:21831580-21921256,

a risk haplotype having chromosome coordinates chr14:48831824-49203827,

a risk haplotype having chromosome coordinates chr5:16071171-16152955,

a risk haplotype having chromosome coordinates chr19:33963105-34145310,

a risk haplotype having chromosome coordinates chr16:43665149-43737129,

a risk haplotype having chromosome coordinates chr15:63767963-63800415,

a risk haplotype having chromosome coordinates chr16:40883517-41081510,

a risk haplotype having chromosome coordinates chr25:43476429-43528145,

a risk haplotype having chromosome coordinates chr1:112977233-113081800,

a risk haplotype having chromosome coordinates chr3:5162058-6465753,

a risk haplotype having chromosome coordinates chr7:64631053-64703475,

a risk haplotype having chromosome coordinates chr1:115582915-116790630,

a risk haplotype having chromosome coordinates chr2:19212450-19542015,

a risk haplotype having chromosome coordinates chr1:122033806-122051988,

a risk haplotype having chromosome coordinates chr35:18326079-18345318,

a risk haplotype having chromosome coordinates chr9:47647012-47668054,

a risk haplotype having chromosome coordinates chr38:11252518-11739329,

a risk haplotype having chromosome coordinates chr21:46231985-46363479,

a risk haplotype having chromosome coordinates chr17:14465884-14482152,

a risk haplotype having chromosome coordinates chr32:25136302-25156153,

a risk haplotype having chromosome coordinates chr36:29637804-29663408,

a risk haplotype having chromosome coordinates chr15:37986345-39974762,

a risk haplotype having chromosome coordinates chr1:29405587-29914411,

a risk haplotype having chromosome coordinates chr26:32374093-32428448,

a risk haplotype having chromosome coordinates chr25:29658978-29767164,

a risk haplotype having chromosome coordinates chr26:3529343-3550075,

a risk haplotype having chromosome coordinates chr5:14720254-15466603,

a risk haplotype having chromosome coordinates chr18:4266743-5854451,

a risk haplotype having chromosome coordinates chr1:16768869-18150476,

a risk haplotype having chromosome coordinates chr9:18896060-19633155, or

a risk haplotype having chromosome coordinates chr11:44390633-44406002.

In some embodiments, the risk haplotype is selected from:

a risk haplotype having chromosome coordinates chr11:39643190-45990018,

a risk haplotype having chromosome coordinates chr24:27409719-29194396, and

a risk haplotype having chromosome coordinates chr35:11233053-12732906. The chromosome coordinates is the previous sentence are from the CanFam3 genome assembly (see, e.g., UCSC Genome Browser).

In some embodiments, the risk haplotype is selected from:

a risk haplotype having chromosome coordinates chr11:37000000-44000000,

a risk haplotype having chromosome coordinates chr24:27000000-33000000, and

a risk haplotype having chromosome coordinates chr35:10000000-14000000. The chromosome coordinates is the previous sentence are from the CanFam3 genome assembly (see, e.g., UCSC Genome Browser).

Any chromosomal coordinates described herein are meant to be inclusive (i.e., include the boundaries of the chromosomal coordinates). In some embodiments, the risk haplotype may include additional chromosomal regions flanking those chromosomal regions described above, e.g., an additional 0.1, 0.5, 1, 2, 3, 4 or 5 Mb. In some embodiments, the risk haplotype may be a shortened chromosomal region than those chromosomal regions described above, e.g., 0.1, 0.5, or 1 Mb fewer than the chromosomal regions described above.

Any mutation of any size located within or spanning the chromosomal boundaries of a risk haplotype is contemplated herein for detection of a risk haplotype, e.g., a SNP, a deletion, an inversion, a translocation, or a duplication. In some embodiments, the risk haplotype is detected by analyzing the chromosomal region of the risk haplotype for the presence of a SNP. In some embodiments, a SNP in a risk haplotype is a SNP described in Table 1 having chromosome coordinates within the risk haplotype. It is to be understood that other SNPs not listed in Table 1 but located within the risk haplotype coordinates on chromosome 1, 2, 3, 5, 7, 8, 9, 11, 13, 14, 15, 16, 17, 18, 19, 21, 25, 26, 32, 35, 36, or 38 above are also contemplated herein. In some embodiments, if the subject is a human subject, then human chromosome coordinates that correspond to canine chromosome coordinates provided herein are contemplated for use in a method described herein.

In some embodiments, a risk haplotype can be used in the methods described herein. In some embodiments, the method comprises:

a) analyzing genomic DNA from a canine subject for the presence of a risk haplotype selected from:

a risk haplotype having chromosome coordinates chr11:44392734-44414985,

a risk haplotype having chromosome coordinates chr8:35433142-35454649,

a risk haplotype having chromosome coordinates chr13:14549973-14645634,

a risk haplotype having chromosome coordinates chr25:21831580-21921256,

a risk haplotype having chromosome coordinates chr14:48831824-49203827,

a risk haplotype having chromosome coordinates chr5:16071171-16152955,

a risk haplotype having chromosome coordinates chr19:33963105-34145310,

a risk haplotype having chromosome coordinates chr16:43665149-43737129,

a risk haplotype having chromosome coordinates chr15:63767963-63800415,

a risk haplotype having chromosome coordinates chr16:40883517-41081510,

a risk haplotype having chromosome coordinates chr25:43476429-43528145,

a risk haplotype having chromosome coordinates chr1:112977233-113081800,

a risk haplotype having chromosome coordinates chr3:5162058-6465753,

a risk haplotype having chromosome coordinates chr7:64631053-64703475,

a risk haplotype having chromosome coordinates chr1:115582915-116790630,

a risk haplotype having chromosome coordinates chr2:19212450-19542015,

a risk haplotype having chromosome coordinates chr1:122033806-122051988,

a risk haplotype having chromosome coordinates chr35:18326079-18345318,

a risk haplotype having chromosome coordinates chr9:47647012-47668054,

a risk haplotype having chromosome coordinates chr38:11252518-11739329,

a risk haplotype having chromosome coordinates chr21:46231985-46363479,

a risk haplotype having chromosome coordinates chr17:14465884-14482152,

a risk haplotype having chromosome coordinates chr32:25136302-25156153,

a risk haplotype having chromosome coordinates chr36:29637804-29663408,

a risk haplotype having chromosome coordinates chr15:37986345-39974762,

a risk haplotype having chromosome coordinates chr1:29405587-29914411,

a risk haplotype having chromosome coordinates chr26:32374093-32428448,

a risk haplotype having chromosome coordinates chr25:29658978-29767164,

a risk haplotype having chromosome coordinates chr26:3529343-3550075,

a risk haplotype having chromosome coordinates chr5:14720254-15466603,

a risk haplotype having chromosome coordinates chr18:4266743-5854451,

a risk haplotype having chromosome coordinates chr1:16768869-18150476,

a risk haplotype having chromosome coordinates chr9:18896060-19633155, and

a risk haplotype having chromosome coordinates chr11:44390633-44406002; and

b) identifying a canine subject having the risk haplotype as a subject (a) at elevated risk of developing osteosarcoma or (b) having an undiagnosed osteosarcoma.

In some embodiments, the risk haplotype is selected from a risk haplotype having chromosome coordinates chr11:44392734-44414985, chr8:35433142-35454649, chr1:115582915-116790630, chr2:19212450-19542015, chr1:122033806-122051988, chr35:18326079-18345318, chr9:47647012-47668054, chr38:11252518-11739329, chr5:14720254-15466603, or chr18:4266743-5854451.

In some embodiments, the risk haplotype is selected from a risk haplotype having chromosome coordinates chr11:44392734-44414985, chr1:115582915-116790630, or chr5:14720254-15466603.

In some embodiments, the risk haplotype is the risk haplotype having chromosome coordinates chr11:44392734-44414985.

In some embodiments, the risk haplotype is the risk haplotype having chromosome coordinates chr11:44390633-44406002.

In some embodiments, the risk haplotype is a risk haplotype having chromosome coordinates chr11:44390000-44410000.

In some embodiments, the method comprises:
a) analyzing genomic DNA from a canine subject for the presence of a risk haplotype selected from:

a risk haplotype having chromosome coordinates chr11:39643190-45990018,

a risk haplotype having chromosome coordinates chr24:27409719-29194396, and

a risk haplotype having chromosome coordinates chr35:11233053-12732906; and

b) identifying a canine subject having the risk haplotype as a subject (a) at elevated risk of developing osteosarcoma or (b) having an undiagnosed osteosarcoma. The chromosome coordinates is the previous sentence are from the CanFam3 genome assembly (see, e.g., UCSC Genome Browser).

In some embodiments, the method comprises:

a) analyzing genomic DNA from a canine subject for the presence of a risk haplotype selected from:

a risk haplotype having chromosome coordinates chr11:37000000-44000000,

a risk haplotype having chromosome coordinates chr24:27000000-33000000, and

a risk haplotype having chromosome coordinates chr35:10000000-14000000; and

b) identifying a canine subject having the risk haplotype as a subject (a) at elevated risk of developing osteosarcoma or (b) having an undiagnosed osteosarcoma. The chromosome coordinates is the previous sentence are from the CanFam3 genome assembly (see, e.g., UCSC Genome Browser).

It is to be understood that any number of mutations (e.g., 1, 2, 3, 4, 5, 6, 7, 8, 9, 10, 11, 12, 13, 14, 15, 16, 17, 18, 19, or 20 or more mutations) can exist within each risk haplotype. It is also to be understood that not all mutations within the risk haplotype must be detected in order to determine that the risk haplotype is present. For example, one mutation may be used to detect the presence of a risk haplotype. In another example, two or more mutations may be used to detect and/or confirm the presence of a risk haplotype. It is also to be understood that subject identification may involve any number of risk haplotypes (e.g., 1, 2, 3, 4, or 5 risk haplotypes).

In some embodiments, the presence of a risk haplotype is determined by detecting one or more SNPs within the chromosomal coordinates of the risk haplotype. In some embodiments, the presence of the risk haplotype is detected by analyzing the genomic DNA for the presence of one or more SNPs in Table 1 within the chromosomal coordinates of the risk haplotype.

It is to be understood that any number of SNPs (e.g., 1, 2, 3, 4, 5, 6, 7, 8, 9, 10, 11, 12, 13, 14, 15, 16, 17, 18, 19, or 20 or more SNPs) in any number of risk haplotypes (e.g., 1, 2, 3, 4, or 5 risk haplotypes) may be used. In some embodiments, a subset or all SNPs in Table 1 located within a risk haplotype are used to detect the presence of the risk haplotype.

### Genes

In some embodiments, a germ-line risk marker is a mutation in a gene. As used herein, a gene includes both coding and non-coding nucleotide sequences. As such, a gene includes any regulatory sequences (e.g., any promoters, enhancers, or suppressors, either adjacent to or far from the coding sequence) and any coding sequences. In some embodiments, a gene includes a nucleotide sequence that encodes a microRNA. In some embodiments, the gene is contained within, near, or spanning the boundaries of a risk haplotype as described herein. In some embodiments, a mutation, such as a SNP, is contained within or near the gene. In some embodiments, the gene is within 1000 Kb, 900 Kb, 800 Kb, 700 Kb, 600 Kb, 500 Kb, 400 Kb, 300 Kb, 200 Kb, or 100 Kb of a SNP as described herein. In some embodiments, the mutation is present in a gene selected from:

one or more genes located within a risk haplotype having chromosome coordinates chr11:44392734-44414985,

one or more genes located within a risk haplotype having chromosome coordinates chr8:35433142-35454649,

one or more genes located within a risk haplotype having chromosome coordinates chr13: 14549973-14645634,

one or more genes located within a risk haplotype having chromosome coordinates chr25:21831580-21921256,

one or more genes located within a risk haplotype having chromosome coordinates chr14:48831824-49203827,

one or more genes located within a risk haplotype having chromosome coordinates chr5:16071171-16152955,

one or more genes located within a risk haplotype having chromosome coordinates chr19:33963105-34145310,

one or more genes located within a risk haplotype having chromosome coordinates chr16:43665149-43737129,

one or more genes located within a risk haplotype having chromosome coordinates chr15:63767963-63800415,

one or more genes located within a risk haplotype having chromosome coordinates chr16:40883517-41081510,

one or more genes located within a risk haplotype having chromosome coordinates chr25:43476429-43528145,

one or more genes located within a risk haplotype having chromosome coordinates chr1:112977233-113081800,

one or more genes located within a risk haploypte having chromosome coordinates chr3:5162058-6465753,

one or more genes located within a risk haplotype having chromosome coordinates chr7:64631053-64703475,

one or more genes located within a risk haplotype having chromosome coordinates chr1:115582915-116790630,

one or more genes located within a risk haplotype having chromosome coordinates chr2:19212450-19542015,

one or more genes located within a risk haplotype having chromosome coordinates chr1:122033806-122051988,

one or more genes located within a risk haplotype having chromosome coordinates chr35: 18326079-18345318,

one or more genes located within a risk haplotype having chromosome coordinates chr9:47647012-47668054,

one or more genes located within a risk haplotype having chromosome coordinates chr38: 11252518-11739329,

one or more genes located within a risk haplotype having chromosome coordinates chr21:46231985-46363479,

one or more genes located within a risk haplotype having chromosome coordinates chr17:14465884-14482152,

one or more genes located within a risk haplotype having chromosome coordinates chr32:25136302-25156153,

one or more genes located within a risk haplotype having chromosome coordinates chr36:29637804-29663408,

one or more genes located within a risk haplotype having chromosome coordinates chr15:37986345-39974762,

one or more genes located within a risk haplotype having chromosome coordinates chr1:29405587-29914411,

one or more genes located within a risk haplotype having chromosome coordinates chr26: 32374093-32428448,

one or more genes located within a risk haplotype having chromosome coordinates chr25:29658978-29767164,

one or more genes located within a risk haplotype having chromosome coordinates chr26:3529343-3550075,

one or more genes located within a risk haplotype having chromosome coordinates chr5:14720254-15466603,

one or more genes located within a risk haplotype having chromosome coordinates chr18:4266743-5854451,

one or more genes located within a risk haplotype having chromosome coordinates chr1:16768869-18150476,

one or more genes located within a risk haplotype having chromosome coordinates chr9:18896060-19633155, or

one or more genes located within a risk haplotype having chromosome coordinates chr11:44390633-44406002.

The mapped genes located within or near the risk haplotypes on chromosome 1, 2, 3, 5, 7, 8, 9, 11, 13, 14, 15, 16, 17, 18, 19, 21, 25, 26, 32, 35, 36, and 38 are described in Table 2 and 3. The Ensembl gene identifiers are based on the CanFam 2.0 genome assembly (see, e.g., Lindblad-Toh K, Wade C M, Mikkelsen T S, Karlsson E K, Jaffe D B, Kamal M, Clamp M, Chang J L, Kulbokas E J 3rd, Zody M C, et al.: Genome sequence, comparative analysis and haplotype structure of the domestic dog. Nature 2005, 438:803-819). The Ensembl gene ID provided for each gene can be used to determine the nucleotide sequence of the gene, as well as associated transcript and protein sequences, by inputting the Ensemble ID into the Ensemble database (Ensembl release 70).

TABLE 2
Genes present in or near chromosomal regions associated with elevated risk of
osteosarcoma

| Gene Symbol | Canine Ensembl ID | Human Ensembl ID | Associated Risk Haplotype |
|---|---|---|---|
| CDKN2B-AS | ENSCAFG00000029763 | ENSG00000240498 | chr11: 44392734 . . . 44414985 |
| OTX2 | ENSCAFG00000015216 | ENSG00000165588 | chr8: 35433142 . . . 35454649 |
| BMPER | ENSCAFG00000003167 | ENSG00000164619 | chr14: 48831824 . . . 49203827 |
| GRIK4 | ENSCAFG00000011880 | ENSG00000149403 | chr5: 16071171 . . . 16152955 |
| EN1 | ENSCAFG00000032481 | ENSG00000163064 | chr19: 33963105 . . . 34145310 |
| MARCO | ENSCAFG00000004913 | ENSG00000019169 | chr19: 33963105 . . . 34145310 |
| MTMR7 | ENSCAFG00000006915 | ENSG00000003987 | chr16: 43665149 . . . 43737129 |
| SGCZ | ENSCAFG00000006792 | ENSG00000185053 | chr16: 40883517 . . . 41081510 |
| CCL20 | ENSCAFG00000010505 | ENSG00000115009 | chr25: 43476429 . . . 43528145 |
| CD3EAP | ENSCAFG00000004455 | ENSG00000117877 | chr1: 112977233 . . . 113081800 |
| ERCC1 | ENSCAFG00000004448 | ENSG00000012061 | chr1: 112977233 . . . 113081800 |
| ERCC2 | ENSCAFG00000004487 | ENSG00000104884 | chr1: 112977233 . . . 113081800 |
| FOSB | ENSCAFG00000004443 | ENSG00000125740 | chr1: 112977233 . . . 113081800 |
| PPP1R13L | ENSCAFG00000004459 | ENSG00000104881 | chr1: 112977233 . . . 113081800 |
| FER | ENSCAFG00000007431 | ENSG00000151422 | chr3: 5162058 . . . 6465753 |
| MAN2A1 | ENSCAFG00000007417 | ENSG00000112893 | chr3: 5162058 . . . 6465753 |
| PJA2 | ENSCAFG00000007425 | ENSG00000198961 | chr3: 5162058 . . . 6465753 |
| CHST9 | ENSCAFG00000018122 | ENSG00000154080 | chr7: 64631053 . . . 64703475 |
| ADCK4 | ENSCAFG00000005108 | ENSG00000123815 | chr1: 115582915 . . . 116790630 |
| AKT2 | ENSCAFG00000005388 | ENSG00000105221 | chr1: 115582915 . . . 116790630 |
| AXL | ENSCAFG00000005041 | ENSG00000167601 | chr1: 115582915 . . . 116790630 |
| BLVRB | NSCAFG00000005338 | ENSG00000090013 | chr1: 115582915 . . . 116790630 |
| C19ORF47 | ENSCAFG00000005372 | ENSG00000160392 | chr1: 115582915 . . . 116790630 |
| C19ORF54 | ENSCAFG00000005103 | ENSG00000188493 | chr1: 115582915 . . . 116790630 |
| CNTD2 | ENSCAFG00000030355 | NSG00000105219 | chr1: 115582915 . . . 116790630 |
| CYP2A7 | ENSCAFG00000031823 | ENSG00000198077 | chr1: 115582915 . . . 116790630 |
| CYP2B6 | ENSCAFG00000005052 | ENSG00000197408 | chr1: 115582915 . . . 116790630 |
| CYP2S1 | ENSCAFG00000005049 | ENSG00000167600 | chr1: 115582915 . . . 116790630 |
| DLL3 | ENSCAFG00000005441 | ENSG00000090932 | chr1: 115582915 . . . 116790630 |
| EGLN2 | ENSCAFG00000005079 | ENSG00000269858 | chr1: 115582915 . . . 116790630 |
| FBL | ENSCAFG00000005412 | ENSG00000105202 | chr1: 115582915 . . . 116790630 |
| FCGBP | ENSCAFG00000005406 | ENSG00000090920 | chr1: 115582915 . . . 116790630 |
| GMFG | ENSCAFG00000028607 | ENSG00000130755 | chr1: 115582915 . . . 116790630 |
| HIPK4 | ENSCAFG00000005355 | ENSG00000160396 | chr1: 115582915 . . . 116790630 |
| HNRNPUL1 | ENSCAFG00000005026 | ENSG00000105323 | chr1: 115582915 . . . 116790630 |
| ITPKC | ENSCAFG00000005104 | ENSG00000086544 | chr1: 115582915 . . . 116790630 |
| LEUTX | ENSCAFG00000028552 | ENSG00000213921 | chr1: 115582915 . . . 116790630 |
| LTBP4 | ENSCAFG00000005133 | ENSG00000090006 | chr1: 115582915 . . . 116790630 |
| MAP3K10 | ENSCAFG00000005393 | ENSG00000130758 | chr1: 115582915 . . . 116790630 |
| MED29 | ENSCAFG00000005533 | ENSG00000063322 | chr1: 115582915 . . . 116790630 |
| NUMBL | ENSCAFG00000005123 | ENSG00000105245 | chr1: 115582915 . . . 116790630 |
| PLD3 | ENSCAFG00000005362 | ENSG00000105223 | chr1: 115582915 . . . 116790630 |
| PLEKHG2 | ENSCAFG00000005521 | ENSG00000090924 | chr1: 115582915 . . . 116790630 |
| PSMC4 | ENSCAFG00000005398 | ENSG00000013275 | chr1: 115582915 . . . 116790630 |
| RAB4B | ENSCAFG00000005083 | ENSG00000167578 | chr1: 115582915 . . . 116790630 |
| | | ENSG00000171570 | |
| SAMD4B | ENSCAFG00000005568 | ENSG00000179134 | chr1: 115582915 . . . 116790630 |
| SERTAD1 | ENSCAFG00000005345 | ENSG00000197019 | chr1: 115582915 . . . 116790630 |
| SERTAD3 | ENSCAFG00000005340 | ENSG00000167565 | chr1: 115582915 . . . 116790630 |
| SHKBP1 | ENSCAFG00000005141 | ENSG00000160410 | chr1: 115582915 . . . 116790630 |
| SNRPA | ENSCAFG00000005091 | ENSG00000077312 | chr1: 115582915 . . . 116790630 |
| SPTBN4 | ENSCAFG00000005286 | NSG00000160460 | chr1: 115582915 . . . 116790630 |
| SUPT5H | ENSCAFG00000005469 | ENSG00000196235 | chr1: 115582915 . . . 116790630 |
| TIMM50 | ENSCAFG00000005445 | ENSG00000105197 | chr1: 115582915 . . . 116790630 |
| KIAA1462 | ENSCAFG00000003987 | ENSG00000165757 | chr2: 19212450 . . . 19542015 |
| C19orf40 | ENSCAFG00000029358 | ENSG00000131944 | chr1: 122033806 . . . 122051988 |
| CEP89 | ENSCAFG00000007486 | ENSG00000121289 | chr1: 122033806 . . . 122051988 |
| RHPN2 | ENSCAFG00000007465 | ENSG00000131941 | chr1: 122033806 . . . 122051988 |
| BLMH | NSCAFG00000019005 | ENSG00000108578 | chr9: 47647012 . . . 47668054 |
| TMIGD1 | ENSCAFG00000019009 | ENSG00000182271 | chr9: 47647012 . . . 47668054 |
| FAM5C | ENSCAFG00000010624 | ENSG00000162670 | chr38: 11252518 . . . 11739329 |
| NELL1 | ENSCAFG00000009933 | ENSG00000165973 | chr21: 46231985 . . . 46363479 |
| EMCN | ENSCAFG00000032716 | ENSG00000164035 | chr32: 25136302 . . . 25156153 |
| AMDHD1 | ENSCAFG00000006406 | ENSG00000139344 | chr15: 37986345 . . . 39974762 |
| CCDC38 | ENSCAFG00000023384 | ENSG00000165972 | chr15: 37986345 . . . 39974762 |
| CDK17 | ENSCAFG00000006459 | ENSG00000059758 | chr15: 37986345 . . . 39974762 |
| ELK3 | NSCAFG00000006454 | ENS000000111145 | chr15: 37986345 . . . 39974762 |
| FGD6 | ENSCAFG00000006273 | ENSG00000180263 | chr15: 37986345 . . . 39974762 |
| HAL | ENSCAFG00000006412 | ENSG00000084110 | chr15: 37986345 . . . 39974762 |
| LTA4H | ENSCAFG00000006440 | ENSG00000111144 | chr15: 37986345 . . . 39974762 |
| METAP2 | ENSCAFG00000006353 | ENSG00000111142 | chr15: 37986345 . . . 39974762 |
| NDUFA12 | ENSCAFG00000006232 | ENSG00000184752 | chr15: 37986345 . . . 39974762 |
| NEDD1 | ENSCAFG00000006509 | ENSG00000139350 | chr15: 37986345 . . . 39974762 |
| NR2C1 | ENSCAFG00000006244 | ENSG00000120798 | chr15: 37986345 . . . 39974762 |
| NTN4 | ENSCAF000000006388 | ENSG00000074527 | chr15: 37986345 . . . 39974762 |
| SNRPF | ENSCAFG00000006395 | ENSG00000139343 | chr15: 37986345 . . . 39974762 |
| USP44 | ENSCAFG00000006375 | ENSG00000136014 | chr15: 37986345 . . . 39974762 |
| VEZT | ENSCAFG00000006331 | ENSG00000028203 | chr15: 37986345 . . . 39974762 |
| EYA4 | ENSCAFG00000000196 | ENSG00000112319 | chr1: 29405587 . . . 29914411 |
| TCF21 | ENSCAF000000000205 | ENSG00000118526 | chr1: 29405587 . . . 29914411 |
| ARVCF | ENSCAFG00000014232 | ENSG00000099889 | chr26: 32374093 . . . 32428448 |
| C22orf25 | ENSCAFG00000014212 | ENSG00000183597 | chr26: 32374093 . . . 32428448 |
| COMT | ENSCAFG00000014253 | ENSG00000093010 | chr26: 32374093 . . . 32428448 |
| XKR6 | ENSCAFG00000008074 | ENSG00000171044 | chr25: 29658978 . . . 29767164 |
| FBRSL1 | ENSCAFG00000023460 | ENSG00000112787 | chr26: 3529343 . . . 3550075 |
| BLID | Orthologue of | ENSG00000259571 | chr5: 14720254 . . . 15466603 |
| | ENSG00000259571 | | |
| C7orf72 | ENSCAFG00000029400 | ENSG00000164500 | chr18: 4266743 . . . 5854451 |
| COBL | ENSCAFG00000003438 | ENSG00000106078 | chr18: 4266743 . . . 5854451 |
| DDC | ENSCAFG00000003400 | ENSG00000132437 | chr18: 4266743 . . . 5854451 |
| FIGNL1 | ENSCAFG00000003379 | ENSG00000132436 | chr18: 4266743 . . . 5854451 |
| GRB10 | ENSCAFG00000003422 | ENSG00000106070 | chr18: 4266743 . . . 5854451 |
| IKZF1 | ENSCAFG00000003374 | ENSG00000185811 | chr18: 4266743 . . . 5854451 |
| VWC2 | ENSCAFG00000003354 | ENSG00000188730 | chr18: 4266743 . . . 5854451 |
| ZPBP | ENSCAFG00000003356 | ENSG00000042813 | chr18: 4266743 . . . 5854451 |
| BCL2 | ENSCAFG00000000068 | ENSG00000171791 | chr1: 16768869 . . . 18150476 |
| KIAA1468 | ENSCAFG00000000079 | ENSG00000134444 | chr1: 16768869 . . . 18150476 |
| PHLPP1 | ENSCAFG00000000070 | ENSG00000081913 | chr1: 16768869 . . . 18150476 |
| PIGN | ENSCAFG00000000083 | ENSG00000197563 | chr1: 16768869 . . . 18150476 |
| RNF152 | ENSCAFG00000032187 | ENSG00000176641 | chr1: 16768869 . . . 18150476 |
| TNFRSF11A | ENSCAFG00000000075 | ENSG00000141655 | chr1: 16768869 . . . 18150476 |
| ZCCHC2 | ENSCAFG00000000073 | ENSG00000141664 | chr1: 16768869 . . . 18150476 |
| ABCA5 | ENSCAF000000010810 | ENSG00000154265 | chr9: 18896060 . . . 19633155 |
| KCNJ16 | ENSCAFG00000010741 | ENSG00000153822 | chr9: 18896060 . . . 19633155 |
| KCNJ2 | ENSCAFG00000010736 | ENSG00000123700 | chr9: 18896060 . . . 19633155 |
| MAP2K6 | ENSCAFG00000010758 | ENSG00000108984 | chr9: 18896060 . . . 19633155 |
| MAFB | ENSCAFG00000024903 | ENSG00000204103 | chr24: 27409719-29194396, |
| | | | chr24: 27000000-33000000 |
| TOP1 | ENSCAFG00000009058 | ENSG00000198900 | chr24: 27409719-29194396, |
| | | | chr24: 27000000-33000000 |
| DHX35 | ENSCAFG00000009044 | ENSG00000101452 | chr24: 27409719-29194396, |
| | | | chr24: 27000000-33000000 |
| ADTRP | ENSCAFG00000009785 | ENSG00000111863 | chr35: 11233053-12732906, |
| | | | chr35: 10000000-14000000 |
| HIVEP1 | ENSCAFG00000009791 | ENSG00000095951 | chr35: 11233053-12732906, |
| | | | chr35: 10000000-14000000 |
| EDN1 | ENSCAFG00000009794 | ENSG00000078401 | chr35: 11233053-12732906, |
| | | | chr35: 10000000-14000000 |
| PHACTR1 | ENSCAFG00000009796 | ENSG00000112137 | chr35: 11233053-12732906, |
| | | | chr35: 10000000-14000000 |

TABLE 3
microRNAs within chromosomal regions associated with elevated risk of
osteosarcoma

| | RefSeq Name | |
|---|---|---|
| Gene Name | (human) | Associated Risk Haplotype |
| MIR100 | NR_029515 | chr5: 14720254 . . . 15466603 |
| MIR100HG | NR_024430 | chr5: 14720254 . . . 15466603 |
| MIR125B1 | NR_029671 | chr5: 14720254 . . . 15466603 |
| MIR331 | NR_029895 | chr15: 37986345 . . . 39974762 |
| MIR3685 | NR_037456 | chr15: 37986345 . . . 39974762 |
| MIR4530 | NR_039755 | chr1: 115582915 . . . 116790630 |
| MIR5684 | NR_049864 | chr15: 63767963 . . . 63800415 |
| MIR641 | NR_030371 | chr1: 115582915 . . . 116790630 |
| MIRLET7A2 | NR_029477 | chr5: 14720254 . . . 15466603 |

In some embodiments, a mutation in a gene is used in the methods described herein. In some embodiments, the method comprises:

(a) analyzing genomic DNA from a canine subject for the presence of a mutation in a gene selected from

one or more genes located within a risk haplotype having chromosome coordinates chr11:44392734-44414985,

one or more genes located within a risk haplotype having chromosome coordinates chr8:35433142-35454649,

one or more genes located within a risk haplotype having chromosome coordinates chr13:14549973-14645634,

one or more genes located within a risk haplotype having chromosome coordinates chr25:21831580-21921256,

one or more genes located within a risk haplotype having chromosome coordinates chr14:48831824-49203827,

one or more genes located within a risk haplotype having chromosome coordinates chr5:16071171-16152955,

one or more genes located within a risk haplotype having chromosome coordinates chr19:33963105-34145310,

one or more genes located within a risk haplotype having chromosome coordinates chr16:43665149-43737129,

one or more genes located within a risk haplotype having chromosome coordinates chr15:63767963-63800415,

one or more genes located within a risk haplotype having chromosome coordinates chr16:40883517-41081510,

one or more genes located within a risk haplotype having chromosome coordinates chr25:43476429-43528145,

one or more genes located within a risk haplotype having chromosome coordinates chr1:112977233-113081800,

one or more genes located within a risk haplotype having chromosome coordinates chr3:5162058-6465753,

one or more genes located within a risk haplotype having chromosome coordinates chr7:64631053-64703475,

one or more genes located within a risk haplotype having chromosome coordinates chr1:115582915-116790630,

one or more genes located within a risk haplotype having chromosome coordinates chr2:19212450-19542015,

one or more genes located within a risk haplotype having chromosome coordinates chr1:122033806-122051988,

one or more genes located within a risk haplotype having chromosome coordinates chr35:18326079-18345318,

one or more genes located within a risk haplotype having chromosome coordinates chr9:47647012-47668054,

one or more genes located within a risk haplotype having chromosome coordinates chr38: 11252518-11739329,

one or more genes located within a risk haplotype having chromosome coordinates chr21:46231985-46363479,

one or more genes located within a risk haplotype having chromosome coordinates chr17: 14465884-14482152,

one or more genes located within a risk haplotype having chromosome coordinates chr32:25136302-25156153,

one or more genes located within a risk haplotype having chromosome coordinates chr36: 29637804-29663408,

one or more genes located within a risk haplotype having chromosome coordinates chr15:37986345-39974762,

one or more genes located within a risk haplotype having chromosome coordinates chr1:29405587-29914411,

one or more genes located within a risk haplotype having chromosome coordinates chr26: 32374093-32428448,

one or more genes located within a risk haplotype having chromosome coordinates chr25:29658978-29767164,

one or more genes located within a risk haplotype having chromosome coordinates chr26:3529343-3550075,

one or more genes located within a risk haplotype having chromosome coordinates chr5:14720254-15466603,

one or more genes located within a risk haplotype having chromosome coordinates chr18:4266743-5854451,

one or more genes located within a risk haplotype having chromosome coordinates chr1:16768869-18150476,

one or more genes located within a risk haplotype having chromosome coordinates chr9:18896060-19633155, and

one or more genes located within a risk haplotype having chromosome coordinates chr11:44390633-44406002; and

(b) identifying a canine subject having the mutation as a subject (a) at elevated risk of developing osteosarcoma or (b) having an undiagnosed osteosarcoma.

In some embodiments, the gene is selected from:

one or more genes located within a risk haplotype having chromosome coordinates chr11:44392734-44414985,

one or more genes located within a risk haplotype having chromosome coordinates chr8:35433142-35454649,

one or more genes located within a risk haplotype having chromosome coordinates chr1:115582915-116790630,

one or more genes located within a risk haplotype having chromosome coordinates chr2:19212450-19542015,

one or more genes located within a risk haplotype having chromosome coordinates chr1:122033806-122051988,

one or more genes located within a risk haplotype having chromosome coordinates chr35: 18326079-18345318,

one or more genes located within a risk haplotype having chromosome coordinates chr9:47647012-47668054,

one or more genes located within a risk haplotype having chromosome coordinates chr38:11252518-11739329,

one or more genes located within a risk haplotype having chromosome coordinates chr5: 14720254-15466603, or

one or more genes located within a risk haplotype having chromosome coordinates chr18:4266743-5854451.

In some embodiments, the gene is selected from:

one or more genes located within a risk haplotype having chromosome coordinates chr11:44392734-44414985,

one or more genes located within a risk haplotype having chromosome coordinates chr1:115582915-116790630, and

one or more genes located within a risk haplotype having chromosome coordinates chr5:14720254-15466603.

In some embodiments, the gene is one or more genes located within the risk haplotype having chromosome coordinates chr11:44392734-44414985.

In some embodiments, the gene is selected from CDKN2B-AS, OTX2, BMPER, GRIK4, EN1, MARCO, MTMR7, SGCZ, CCL20, CD3EAP, ERCC1, ERCC2, FOSB, PPP1R13L, FER, MAN2A1, PJA2, CHST9, ADCK4, AKT2, AXL, BLVRB, C19orf47, C19orf54, CNTD2, CYP2A7, CYP2B6, CYP2S1, DLL3, EGLN2, FBL, FCGBP, GMFG, HIPK4, HNRNPUL1, ITPKC, LEUTX, LTBP4, MAP3K10, MED29, NUMBL, PLD3, PLEKHG2, PSMC4, RAB4B, SAMD4B, SERTAD1, SERTAD3, SHKBP1, SNRPA, SPTBN4, SUPT5H, TIMM50, KIAA1462, Cl9orf40, CEP89, RHPN2, BLMH, TMIGD1, FAM5C, NELL1, EMCN, AMDHD1, CCDC38, CDK17, ELK3, FGD6, HAL, LTA4H, METAP2, NDUFA12, NEDD1, NR2C1, NTN4, SNRPF, USP44,VEZT, EYA4, TCF21, ARVCF, C22orf25, COMT, XKR6, FBRSL1, BLID, C7orf72, COBL, DDC, FIGNL1, GRB10, IKZF1, VWC2, ZPBP, BCL2, KIAA1468, PHLPP1, PIGN, RNF152, TNFRSF11A, ZCCHC2, ABCA5, KCNJ16, KCNJ2, MAP2K6, CDKN2A, and CDKN2B.

In some embodiments, the gene is selected from CDKN2B-AS, OTX2, BMPER, EN1, DLL3, KIAA1462, FAM5C, NELL1, EMCN, TCF21, BLID, VWC2, BCL2, and TNFRSF11A.

In some embodiments, the gene is selected from CDKN2B-AS, OTX2, ADCK4, AKT2, AXL, BLVRB, C19orf47, C19orf54, CNTD2, CYP2A7, CYP2B6, CYP2S1, DLL3, EGLN2, FBL, FCGBP, GMFG, HIPK4, HNRNPUL1, ITPKC, LEUTX, LTBP4, MAP3K10, MED29, NUMBL, PLD3, PLEKHG2, PSMC4, RAB4B, SAMD4B, SERTAD1, SERTAD3, SHKBP1, SNRPA, SPTBN4, SUPT5H, TIMM50, KIAA1462, Cl9orf40, CEP89, RHPN2, BLMH, TMIGD1, FAM5C, BLID, C7orf72, COBL, DDC, FIGNL1, GRB10, IKZF1, VWC2, and ZPBP.

In some embodiments, the gene is selected from CDKN2B-AS, ADCK4, AKT2, AXL, BLVRB, C19orf47, C19orf54, CNTD2, CYP2A7, CYP2B6, CYP2S1, DLL3, EGLN2, FBL, FCGBP, GMFG, HIPK4, HNRNPUL1, ITPKC, LEUTX, LTBP4, MAP3K10, MED29, NUMBL, PLD3, PLEKHG2, PSMC4, RAB4B, SAMD4B, SERTAD1, SERTAD3, SHKBP1, SNRPA, SPTBN4, SUPT5H, TIMM50, and BLID.

In some embodiments, the gene is selected from CDKN2B-AS, CDKN2A, and CDKN2B. In some embodiments, the gene is selected from CDKN2B-AS, CDKN2A, CDKN2B, and MTAP.

Any number of mutations (e.g., 1, 2, 3, 4, 5, 6, 7, 8, 9, 10, 11, 12, 13, 14, 15, 16, 17, 18, 19, or 20 or more mutations) in any number of genes (e.g., 1, 2, 3, 4, 5, 6, 7, 8, 9, 10, 11, 12, 13, 14, 15, 16, 17, 18, 19, or 20 or more genes) are contemplated.

The genes described herein can also be used to identify a subject at elevated risk of or having undiagnosed osteosarcoma, where the subject is any of a variety of animal subjects including but not limited to human subjects. In some embodiments, the method, comprises

(a) analyzing genomic DNA in a sample from a subject for presence of a mutation in a gene selected from:

one or more genes located within a risk haplotype having chromosome coordinates chr11:44392734-44414985 or an orthologue of such a gene,

one or more genes located within a risk haplotype having chromosome coordinates chr8:35433142-35454649 or an orthologue of such a gene,

one or more genes located within a risk haplotype having chromosome coordinates chr13:14549973-14645634 or an orthologue of such a gene,

one or more genes located within a risk haplotype having chromosome coordinates chr25:21831580-21921256 or an orthologue of such a gene,

one or more genes located within a risk haplotype having chromosome coordinates chr14:48831824-49203827 or an orthologue of such a gene,

one or more genes located within a risk haplotype having chromosome coordinates chr5:16071171-16152955 or an orthologue of such a gene,

one or more genes located within a risk haplotype having chromosome coordinates chr19:33963105-34145310 or an orthologue of such a gene,

one or more genes located within a risk haplotype having chromosome coordinates chr16:43665149-43737129 or an orthologue of such a gene,

one or more genes located within a risk haplotype having chromosome coordinates chr15:63767963-63800415 or an orthologue of such a gene,

one or more genes located within a risk haplotype having chromosome coordinates chr16:40883517-41081510 or an orthologue of such a gene,

one or more genes located within a risk haplotype having chromosome coordinates chr25:43476429-43528145 or an orthologue of such a gene,

one or more genes located within a risk haplotype having chromosome coordinates chr1:112977233-113081800 or an orthologue of such a gene,

one or more genes located within a risk haplotype having chromosome coordinates chr3:5162058-6465753 or an orthologue of such a gene,

one or more genes located within a risk haplotype having chromosome coordinates chr7:64631053-64703475 or an orthologue of such a gene,

one or more genes located within a risk haplotype having chromosome coordinates chr1:115582915-116790630 or an orthologue of such a gene,

one or more genes located within a risk haplotype having chromosome coordinates chr2:19212450-19542015 or an orthologue of such a gene,

one or more genes located within a risk haplotype having chromosome coordinates chr1:122033806-122051988 or an orthologue of such a gene,

one or more genes located within a risk haplotype having chromosome coordinates chr35:18326079-18345318 or an orthologue of such a gene,

one or more genes located within a risk haplotype having chromosome coordinates chr9:47647012-47668054 or an orthologue of such a gene,

one or more genes located within a risk haplotype having chromosome coordinates chr38:11252518-11739329 or an orthologue of such a gene,

one or more genes located within a risk haplotype having chromosome coordinates chr21:46231985-46363479 or an orthologue of such a gene,

one or more genes located within a risk haplotype having chromosome coordinates chr17:14465884-14482152 or an orthologue of such a gene,

one or more genes located within a risk haplotype having chromosome coordinates chr32:25136302-25156153 or an orthologue of such a gene,

one or more genes located within a risk haplotype having chromosome coordinates chr36:29637804-29663408 or an orthologue of such a gene,

one or more genes located within a risk haplotype having chromosome coordinates chr15:37986345-39974762 or an orthologue of such a gene,

one or more genes located within a risk haplotype having chromosome coordinates chr1:29405587-29914411 or an orthologue of such a gene,

one or more genes located within a risk haplotype having chromosome coordinates chr26:32374093-32428448 or an orthologue of such a gene,

one or more genes located within a risk haplotype having chromosome coordinates chr25:29658978-29767164 or an orthologue of such a gene,

one or more genes located within a risk haplotype having chromosome coordinates chr26:3529343-3550075 or an orthologue of such a gene,

one or more genes located within a risk haplotype having chromosome coordinates chr5:14720254-15466603 or an orthologue of such a gene,

one or more genes located within a risk haplotype having chromosome coordinates chr18:4266743-5854451 or an orthologue of such a gene,

one or more genes located within a risk haplotype having chromosome coordinates chr1:16768869-18150476 or an orthologue of such a gene,

one or more genes located within a risk haplotype having chromosome coordinates chr9:18896060-19633155 or an orthologue of such a gene, and

one or more genes located within a risk haplotype having chromosome coordinates chr11:44,390,633-44,406,002 or an orthologue of such a gene; and

(b) identifying a subject having the mutation as a subject (a) at elevated risk of developing osteosarcoma or (b) having an undiagnosed osteosarcoma. In some embodiments, the subject is a human subject. In some embodiments, the subject is a canine subject. An orthologue of a gene may be, e.g., a human gene as identified in Table 2 or 3. In some embodiments, an orthologue of a gene has a sequence that is 70%, 75%, 80%, 85%, 90%, 95%, or 99% or more homologous to a sequence of the gene.

### Genome Analysis Methods

Some methods provided herein comprise analyzing genomic DNA. In some embodiments, analyzing genomic DNA comprises carrying out a nucleic acid-based assay, such as a sequencing-based assay or a hybridization based assay. In some embodiments, the genomic DNA is analyzed using a single nucleotide polymorphism (SNP) array. In some embodiments, the genomic DNA is analyzed using a bead assay. Methods of genetic analysis are known in the art. Examples of genetic analysis methods and commercially available tools are described below.

Affymetrix: The Affymetrix SNP 6.0 array contains over 1.8 million SNP and copy number probes on a single array. The method utilizes at a simple restriction enzyme digestion of 250 ng of genomic DNA, followed by linker-ligation of a common adaptor sequence to every fragment, a tactic that allows multiple loci to be amplified using a single primer complementary to this adaptor. Standard PCR then amplifies a predictable size range of fragments, which converts the genomic DNA into a sample of reduced complexity as well as increases the concentration of the fragments that reside within this predicted size range. The target is fragmented, labeled with biotin, hybridized to microarrays, stained with streptavidin-phycoerythrin and scanned. To support this method, Affymetrix Fluidics Stations and integrated GS-3000 Scanners can be used.

Illumina Infinium: Examples of commercially available Infinium array options include the 660W-Quad (>660,000 probes), the 1MDuo (over 1 million probes), and the custom iSelect (up to 200,000 SNPs selected by user). Samples begin the process with a whole genome amplification step, then 200 ng is transferred to a plate to be denatured and neutralized, and finally plates are incubated overnight to amplify. After amplification the samples are enzymatically fragmented using end-point fragmentation. Precipitation and resuspension clean up the DNA before hybridization onto the chips. The fragmented, resuspended DNA samples are then dispensed onto the appropriate BeadChips and placed in the hybridization oven to incubate overnight. After hybridization the chips are washed and labeled nucleotides are added to extend the primers by one base. The chips are immediately stained and coated for protection before scanning. Scanning is done with one of the two Illumina iScan™ Readers, which use a laser to excite the fluorophore of the single-base extension product on the beads. The scanner records high-resolution images of the light emitted from the fluorophores. All plates and chips are barcoded and tracked with an internally derived laboratory information management system. The data from these images are analyzed to determine SNP genotypes using Illumina's BeadStudio. To support this process, Biomek F/X, three Tecan Freedom Evos, and two Tecan Genesis Workstation 150s can be used to automate all liquid handling steps throughout the sample and chip prep process.

Illumina BeadArray: The Illumina Bead Lab system is a multiplexed array-based format. Illumina's BeadArray Technology is based on 3-micron silica beads that self-assemble in microwells on either of two substrates: fiber optic bundles or planar silica slides. When randomly assembled on one of these two substrates, the beads have a uniform spacing of ˜5.7 microns. Each bead is covered with hundreds of thousands of copies of a specific oligonucleotide that act as the capture sequences in one of Illumina's assays. BeadArray technology is utilized in Illumina's iScan System.

Sequenom: During pre-PCR, either of two Packard Multiprobes is used to pool oligonucleotides, and a Tomtec Quadra 384 is used to transfer DNA. A Cartesian nanodispenser is used for small-volume transfer in pre-PCR, and another in post-PCR. Beckman Multimeks, equipped with either a 96-tip head or a 384-tip head, are used for more substantial liquid handling of mixes. Two Sequenom pin-tool are used to dispense nanoliter volumes of analytes onto target chips for detection by mass spectrometry. Sequenom Compact mass spectrometers can be used for genotype detection.

In some embodiments, methods provided herein comprise analyzing genomic DNA using a nucleic acid sequencing assay. Methods of genome sequencing are known in the art. Examples of genome sequencing methods and commercially available tools are described below.

Illumina Sequencing: 89 GAIIx Sequencers are used for sequencing of samples. Library construction is supported with 6 Agilent Bravo plate-based automation, Stratagene MX3005p qPCR machines, Matrix 2-D barcode scanners on all automation decks and 2 Multimek Automated Pipettors for library normalization.

454 Sequencing: Roche® 454 FLX-Titanium instruments are used for sequencing of samples. Library construction capacity is supported by Agilent Bravo automation deck, Biomek FX and Janus PCR normalization.

SOLiD Sequencing: SOLiD v3.0 instruments are used for sequencing of samples. Sequencing set-up is supported by a Stratagene MX3005p qPCR machine and a Beckman SC Quanter for bead counting.

ABI Prism® 3730 XL Sequencing: ABI Prism® 3730 XL machines are used for sequencing samples. Automated Sequencing reaction set-up is supported by 2 Multimek

Automated Pipettors and 2 Deerac Fluidics—Equator systems. PCR is performed on 60 Thermo-Hybaid 384-well systems.

Ion Torrent: Ion PGM™ or Ion Proton™ machines are used for sequencing samples. Ion library kits (Invitrogen) can be used to prepare samples for sequencing.

Other Technologies: Examples of other commercially available platforms include Helicos Heliscope Single-Molecule Sequencer, Polonator G.007, and Raindance RDT 1000 Rainstorm.

### Expression Level Analysis

The invention contemplates that elevated risk of developing osteosarcoma is associated with an altered expression pattern of a gene located at, within, or near a risk haplotype, such as a gene located in Table 2 or 3. The invention therefore contemplates methods that involve measuring the mRNA or protein levels for these genes and comparing such levels to control levels, including for example predetermined thresholds.

mRNA Assays

The art is familiar with various methods for analyzing mRNA levels. Examples of mRNA-based assays include but are not limited to oligonucleotide microarray assays, quantitative RT-PCR, Northern analysis, and multiplex bead-based assays.

Expression profiles of cells in a biological sample (e.g., blood or a tumor) can be carried out using an oligonucleotide microarray analysis. As an example, this analysis may be carried out using a commercially available oligonucleotide microarray or a custom designed oligonucleotide microarray comprising oligonucleotides for all or a subset of the transcripts described herein. The microarray may comprise any number of the transcripts, as the invention contemplates that elevated risk may be determined based on the analysis of single differentially expressed transcripts or a combination of differentially expressed transcripts. The transcripts may be those that are up-regulated in tumors carrying a germ-line risk marker (compared to a tumor that does not carry the germ-line risk marker), or those that are down-regulated in tumors carrying a germ-line risk marker (compared to a tumor that does not carry the germ-line risk marker), or a combination of these. The number of transcripts measured using the microarray therefore may be 1, 2, 3, 4, 5, 6, 7, 8, 9, or more transcripts encoded by a gene in Table 2 or 3. It is to be understood that such arrays may however also comprise positive and/or negative control transcripts such as housekeeping genes that can be used to determine if the array has been degraded and/or if the sample has been degraded or contaminated. The art is familiar with the construction of oligonucleotide arrays.

Commercially available gene expression systems include Affymetrix GeneChip microarrays as well as all of IIlumina standard expression arrays, including two GeneChip 450 Fluidics Stations and a GeneChip 3000 Scanner, Affymetrix High-Throughput Array (HTA) System composed of a GeneStation liquid handling robot and a GeneChip HT Scanner providing automated sample preparation, hybridization, and scanning for 96-well Affymetrix PEGarrays. These systems can be used in the cases of small or potentially degraded RNA samples. The invention also contemplates analyzing expression levels from fixed samples (as compared to freshly isolated samples). The fixed samples include formalin-fixed and/or paraffin-embedded samples. Such samples may be analyzed using the whole genome Illumina DASL assay. High-throughput gene expression profile analysis can also be achieved using bead-based solutions, such as Luminex systems.

Other mRNA detection and quantitation methods include multiplex detection assays known in the art, e.g., xMAP® bead capture and detection (Luminex Corp., Austin, Tex.).

Another exemplary method is a quantitative RT-PCR assay which may be carried out as follows: mRNA is extracted from cells in a biological sample (e.g., blood or a tumor) using the RNeasy kit (Qiagen). Total mRNA is used for subsequent reverse transcription using the SuperScript III First-Strand Synthesis SuperMix (Invitrogen) or the SuperScript VILO cDNA synthesis kit (Invitrogen). 5 μl of the RT reaction is used for quantitative PCR using SYBR Green PCR Master Mix and gene-specific primers, in triplicate, using an ABI 7300 Real Time PCR System.

mRNA detection binding partners include oligonucleotide or modified oligonucleotide (e.g. locked nucleic acid) probes that hybridize to a target mRNA. Probes may be designed using the sequences or sequence identifiers listed in Table 2 or 3. Methods for designing and producing oligonucleotide probes are well known in the art (see, e.g., U.S. Pat. No. 8,036,835; Rimour et al. GoArrays: highly dynamic and efficient microarray probe design. Bioinformatics (2005) 21 (7): 1094-1103; and Wernersson et al. Probe selection for DNA microarrays using OligoWiz. Nat Protoc. 2007; 2(11):2677-91).

Protein Assays

The art is familiar with various methods for measuring protein levels. Protein levels may be measured using protein-based assays such as but not limited to immunoassays, Western blots, Western immunoblotting, multiplex bead-based assays, and assays involving aptamers (such as SOMAmer™ technology) and related affinity agents.

A brief description of an exemplary immunoassay is provided here. A biological sample is applied to a substrate having bound to its surface protein-specific binding partners (i.e., immobilized protein-specific binding partners). The protein-specific binding partner (which may be referred to as a “capture ligand” because it functions to capture and immobilize the protein on the substrate) may be an antibody or an antigen-binding antibody fragment such as Fab, F(ab)2, Fv, single chain antibody, Fab and sFab fragment, F(ab′)₂, Fd fragments, scFv, and dAb fragments, although it is not so limited. Other binding partners are described herein. Protein present in the biological sample bind to the capture ligands, and the substrate is washed to remove unbound material. The substrate is then exposed to soluble protein-specific binding partners (which may be identical to the binding partners used to immobilize the protein). The soluble protein-specific binding partners are allowed to bind to their respective proteins immobilized on the substrate, and then unbound material is washed away. The substrate is then exposed to a detectable binding partner of the soluble protein-specific binding partner. In one embodiment, the soluble protein-specific binding partner is an antibody having some or all of its Fc domain. Its detectable binding partner may be an anti-Fc domain antibody. As will be appreciated by those in the art, if more than one protein is being detected, the assay may be configured so that the soluble protein-specific binding partners are all antibodies of the same isotype. In this way, a single detectable binding partner, such as an antibody specific for the common isotype, may be used to bind to all of the soluble protein-specific binding partners bound to the substrate.

It is to be understood that the substrate may comprise capture ligands for one or more proteins, including two or more, three or more, four or more, five or more, etc. up to and including all of the proteins encoded by the genes in Table 2 provided by the invention.

Other examples of protein detection and quantitation methods include multiplexed immunoassays as described for example in U.S. Pat. Nos. 6,939,720 and 8,148,171, and published US Patent Application No. 2008/0255766, and protein microarrays as described for example in published US Patent Application No. 2009/0088329.

Protein detection binding partners include protein-specific binding partners. Protein-specific binding partners can be generated using the sequences or sequence identifiers listed in Table 2. In some embodiments, binding partners may be antibodies. As used herein, the term “antibody” refers to a protein that includes at least one immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab and sFab fragments, F(ab′)₂, Fd fragments, Fv fragments, scFv, and dAb fragments) as well as complete antibodies. Methods for making antibodies and antigen-binding fragments are well known in the art (see, e.g. Sambrook et al, “Molecular Cloning: A Laboratory Manual” (2nd Ed.), Cold Spring Harbor Laboratory Press (1989); Lewin, “Genes IV”, Oxford University Press, New York, (1990), and Roitt et al., “Immunology” (2nd Ed.), Gower Medical Publishing, London, New York (1989), WO2006/040153, WO2006/122786, and WO2003/002609).

Binding partners also include non-antibody proteins or peptides that bind to or interact with a target protein, e.g., through non-covalent bonding. For example, if the protein is a ligand, a binding partner may be a receptor for that ligand. In another example, if the protein is a receptor, a binding partner may be a ligand for that receptor. In yet another example, a binding partner may be a protein or peptide known to interact with a protein. Methods for producing proteins are well known in the art (see, e.g. Sambrook et al, “Molecular Cloning: A Laboratory Manual” (2nd Ed.), Cold Spring Harbor Laboratory Press (1989) and Lewin, “Genes IV”, Oxford University Press, New York, (1990)) and can be used to produce binding partners such as ligands or receptors.

Binding partners also include aptamers and other related affinity agents. Aptamers include oligonucleic acid or peptide molecules that bind to a specific target. Methods for producing aptamers to a target are known in the art (see, e.g., published US Patent Application No. 2009/0075834, U.S. Pat. Nos. 7,435,542, 7,807,351, and 7,239,742). Other examples of affinity agents include SOMAmer™ (Slow Off-rate Modified Aptamer, SomaLogic, Boulder, Colo.) modified nucleic acid-based protein binding reagents.

Binding partners also include any molecule capable of demonstrating selective binding to any one of the target proteins disclosed herein, e.g., peptoids (see, e.g., Reyna J Simon et al., “Peptoids: a modular approach to drug discovery” Proceedings of the National Academy of Sciences USA, (1992), 89(20), 9367-9371; U.S. Pat. No. 5,811,387; and M. Muralidhar Reddy et al., Identification of candidate IgG biomarkers for Alzheimer's disease via combinatorial library screening. Cell 144, 132-142, Jan. 7, 2011).

### Detectable Labels

Detectable binding partners may be directly or indirectly detectable. A directly detectable binding partner may be labeled with a detectable label such as a fluorophore. An indirectly detectable binding partner may be labeled with a moiety that acts upon (e.g., an enzyme or a catalytic domain) or a moiety that is acted upon (e.g., a substrate) by another moiety in order to generate a detectable signal. Exemplary detectable labels include, e.g., enzymes, radioisotopes, haptens, biotin, and fluorescent, luminescent and chromogenic substances. These various methods and moieties for detectable labeling are known in the art.

### Devices and Kits

Any of the methods provided herein can be performed on a device, e.g., an array. Suitable arrays are described herein and known in the art. Accordingly, a device, e.g., an array, for detecting any of the germ-line risk markers (e.g., 1, 2, 3, 4, 5, 6, 7, 8, 9, 10, or more germ-line risk markers, or at least 10, at least 20, at least 30, at least 40, at least 50, or more germ-line risk markers, or up to 5, up to 10, up to 15, up to 20, up to 25, up to 30, up to 35, up to 40, up to 45, up to 50, up to 75 or up to 100 germ-line risk markers) described herein is also contemplated.

Reagents for use in any of the methods provided herein can be in the form of a kit. Accordingly, a kit for detecting any of the germ-line risk markers (e.g., 1, 2, 3, 4, 5, 6, 7, 8, 9, 10, or more germ-line risk markers, or at least 10, at least 20, at least 30, at least 40, at least 50, or more germ-line risk markers, or up to 5, up to 10, up to 15, up to 20, up to 25, up to 30, up to 35, up to 40, up to 45, up to 50, up to 75 or up to 100 germ-line risk markers) described herein is also contemplated. In some embodiments, the kit comprises reagents for detecting any of the germ-line risk markers described herein, e.g., reagents for use in a method described herein. Suitable reagents are described herein and art known in the art.

### Controls

Some of the methods provided herein involve measuring a level or determining the identity of a germ-line risk marker in a biological sample and then comparing that level or identity to a control in order to identify a subject having an elevated risk of developing osteosarcoma or having as yet undiagnosed osteosarcoma. The control may be a control level or identity that is a level or identity of the same germ-line marker in a control tissue, control subject, or a population of control subjects.

The control may be (or may be derived from) a normal subject (or normal subjects). A normal subject, as used herein, refers to a subject that is healthy, such a subject experiencing none of the symptoms associate with osteosarcoma. The control population may be a population of normal subjects.

In other instances, the control may be (or may be derived from) a subject (a) having a similar cancer to that of the subject being tested and (b) who is negative for the germ-line risk marker.

It is to be understood that the methods provided herein do not require that a control level or identity be measured every time a subject is tested. Rather, it is contemplated that control levels or identities of germ-line risk markers are obtained and recorded and that any test level is compared to such a pre-determined level or identity (or threshold).

In some embodiments, a control is a nucleotide other than the risk nucleotide as described in Table 1.

### Samples

The methods provided herein detect and optionally measure (and thus analyze) levels or particular germ-line risk markers in biological samples. Biological samples, as used herein, refer to samples taken or obtained from a subject. These biological samples may be tissue samples or they may be fluid samples (e.g., bodily fluid). Examples of biological fluid samples are whole blood, plasma, serum, urine, sputum, phlegm, saliva, tears, and other bodily fluids. In some embodiments, the biological sample is a whole blood or saliva sample. In some embodiments, the biological sample is a tumor, a fragment of a tumor, or a tumor cell(s). In some embodiments, the biological sample is a bone sample or bone biopsy.

In some embodiments, the biological sample may comprise a polynucleotide (e.g., genomic DNA or mRNA) derived from a tissue sample or fluid sample of the subject. In some embodiments, the biological sample may comprise a polypeptide (e.g., a protein) derived from a tissue sample or fluid sample of the subject. In some embodiments, the biological sample may be manipulated to extract a polynucleotide or polypeptide. In some embodiments, the biological sample may be manipulated to amplify a polynucleotide sample. Methods for extraction and amplification are well known in the art.

### Subjects

Methods of the invention are intended for canine subjects. In some embodiments, canine subjects include, for example, those with a higher incidence of osteosarcoma as determined by breed. For example, the canine subject may be a Irish Wolfhound, Greyhound, German Shepherd, Rottweiler, Great Pyrenees, St. Bernard, Leonberger, Newfoundland, Doberman Pinscher or Great Dane, or a descendant of a Irish Wolfhound, Greyhound, German Shepherd, Rottweiler, Great Pyrenees, St. Bernard, Leonberger, Newfoundland, Doberman Pinscher or Great Dane. In some embodiments, the canine subject may be a Greyhound, an Irish Wolfhound, or a Rottweiler, or a descendant of a Greyhound, an Irish Wolfhound, or a Rottweiler. As used herein, a “descendant” includes any blood relative in the line of descent, e.g., first generation, second generation, third generation, fourth generation, etc., of a canine subject. Such a descendant may be a pure-bred canine subject, e.g., a descendant of two Greyhound or a mixed-breed canine subject, e.g., a descendant of both a Greyhound and a non-Greyhound. Breed can be determined, e.g., using commercially available genetic tests (see, e.g., Wisdom Panel).

Methods of the invention may be used in a variety of other subjects including but not limited to human subjects.

### Computational Analysis

Methods of computation analysis of genomic and expression data are known in the art. Examples of available computational programs are: Genome Analysis Toolkit (GATK, Broad Institute, Cambridge, Mass.), Expressionist Refiner module (Genedata AG, Basel, Switzerland), GeneChip—Robust Multichip Averaging (CG-RMA) algorithm, PLINK (Purcell et al, 2007), GCTA (Yang et al, 2011), the EIGENSTRAT method (Price et al 2006), EMMAX (Kang et al, 2010). In some embodiments, methods described herein include a step comprising computational analysis.

### Breeding Programs

Other aspects of the invention relate to use of the diagnostic methods in connection with a breeding program. A breeding program is a planned, intentional breeding of a group of animals to reduce detrimental or undesirable traits and/or increase beneficial or desirable traits in offspring of the animals. Thus, a subject identified using the methods described herein as not having a germ-line risk marker of the invention may be included in a breeding program to reduce the risk of developing osteosarcoma in the offspring of said subject. Alternatively, a subject identified using the methods described herein as having a germ-line risk marker of the invention may be excluded from a breeding program. In some embodiments, methods of the invention comprise exclusion of a subject identified as being at elevated risk of developing osteosarcoma or having undiagnosed osteosarcoma in a breeding program or inclusion of a subject identified as not being at elevated risk of developing osteosarcoma or having undiagnosed osteosarcoma in a breeding program.

### Treatment

Other aspects of the invention relate to diagnostic or prognostic methods that comprise a treatment step (also referred to as “theranostic” methods due to the inclusion of the treatment step). Any treatment for osteosarcoma is contemplated. In some embodiments, treatment comprises one or more of surgery, chemotherapy, and radiation.

In some embodiments, treatment comprises amputation or limb-salvage surgery. Amputation includes removal of a region of or the entirety of a limb containing the osteosarcoma Limb-salvage surgery includes removal of the bone containing the osteosarcoma and a region of healthy bone and/or tissue surrounding the osteosarcoma (e.g., about an inch around the osteosarcoma). The removed bone is then replaced. The replacement can be, for example, a synthetic rod or plate (prostheses), a piece of bone (graft) taken from the subject's own body (autologous transplant), or a piece of bone removed from a donor body (such as a cadaver) and frozen until needed for transplant (allogeneic transplant).

In some embodiments, treatment comprises administration of an effective amount of mifamurtide, methotrexate, cisplatin, carboplatin, doxyrubicin, adriamycin, ifosfamide, mesna, BCD (bleomycin, cyclophosphamide, dactinomycin), etoposide, muramyl tri-peptite (MTP), alendronate and/or pamidronate. In some embodiments, treatment comprises administration of an effective amount of a chemosensitizer such as suramin.

In some embodiments, treatment comprises administration of an effective amount of ADXS-HER2 (Advaxis). ADXS-HER2 comprises a live, attenuated strain of Listeria containing multiple copies of a plasmid that encodes a fusion protein sequence including a fragment of the LLO (listeriolysin O) molecule joined to HER2.

In some embodiments, treatment comprises apSTAR (autologous patient specific tumor antigen response) Veterinary Cancer Laser System (IMULAN BioTherapeutics, LLC and Veterinary Cancer Therapeutics, LLC). Also known as laser-assisted immunotherapy, apSTAR is a cancer treatment for solid tumors that utilizes an autologous vaccine-like approach to stimulate immune responses. apSTAR combines laser-induced in situ tumor devitalization with an immunoadjuvant for local immunostimulation.

In some embodiments, treatment comprises surgery to remove the primary tumor(s) followed administration of an effective amount of an adjuvant chemotherapy to remove metastatic cells. In some embodiments, treatment further comprises additional adjuvant therapy, such as administration of suramin.

In some embodiment, treatment is palliative treatment. In some embodiments, palliative treatment comprises radiation and/or administration of an effective amount of an analgesic (e.g., an non-steroidal anti-inflammatory drug, NSAID).

It is to be understood that any treatment described herein may be used alone or may be used in combination with any other treatment described herein. In some embodiments, treatment comprises surgery and at least one other therapy, such as chemotherapy or radiation.

In some embodiments, a subject identified as being at elevated risk of developing osteosarcoma or having undiagnosed osteosarcoma is treated. In some embodiments, the method comprises selecting a subject for treatment on the basis of the presence of one or more germ-line risk markers as described herein. In some embodiments, the method comprises treating a subject with osteosarcoma characterized by the presence of one or more germ-line risk markers as defined herein.

As used herein, “treat” or “treatment” includes, but is not limited to, preventing or reducing the development of a cancer, reducing the symptoms of cancer, suppressing or inhibiting the growth of a cancer, preventing metastasis and/or invasion of an existing cancer, promoting or inducing regression of the cancer, inhibiting or suppressing the proliferation of cancerous cells, reducing angiogenesis and/or increasing the amount of apoptotic cancer cells.

An effective amount is a dosage of a therapy sufficient to provide a medically desirable result, such as treatment of cancer. The effective amount will vary with the location of the cancer being treated, the age and physical condition of the subject being treated, the severity of the condition, the duration of the treatment, the nature of any concurrent therapy, the specific route of administration and the like factors within the knowledge and expertise of the health practitioner.

Administration of a treatment may be accomplished by any method known in the art (see, e.g., Harrison's Principle of Internal Medicine, McGraw Hill Inc.). Administration may be local or systemic. Administration may be parenteral (e.g., intravenous, subcutaneous, or intradermal) or oral. Compositions for different routes of administration are well known in the art (see, e.g., Remington's Pharmaceutical Sciences by E. W. Martin). Dosage will depend on the subject and the route of administration. Dosage can be determined by the skilled artisan.

### EXAMPLES

### Example 1

Osteosarcoma in dogs is a spontaneously occurring disease with a global tumor gene expression signature indistinguishable from tumors from human pediatric patients and, while age of onset is higher in dogs, the clinical progression is remarkably similar. Both human and canine osteosarcomas most commonly arise at the ends of the long bones of the limbs and metastasize readily, usually to the lungs. Unlike human osteosarcoma, canine osteosarcoma is primarily a heritable disease affecting primarily large dogs. Particular dog breeds show more than 10-fold increased risk, including the Greyhound (mortality from osteosarcoma=26%), Rottweiler (mortality from osteosarcoma=17%) and Irish Wolfhound (mortality from osteosarcoma=21% [ref. 6-8].

Mapping disease genes using genome wide association study (GWAS) in dog breeds, each effectively a genetic isolate only a few hundred years old, requires approximately 10x fewer markers and samples that in human populations. However, population structure, cryptic relatedness and extensive regions of near fixation in breeds complicate GWAS analysis, and to date just a handful of studies have successfully mapped risk factors for complex, multigenic canine disorders. As described herein, novel methods for analyzing breed populations were used to identify genomic loci explaining the majority of the osteosarcoma phenotype variance in three breed populations, and to uncover novel genes and pathways potentially underlying this poorly understood disease.

### Population Genetics of GWAS Breeds

304 Greyhounds (Grey; 118 Unaffected (U)+186 Affected (A)), 155 Irish wolfhounds (IWH; 68 U+87 A), 145 Rottweilers (Rott; 59 U+86 A) and 14 non-racing AKC registered greyhounds (AKC Grey) were genotyped on the Illumina canineHD SNP arrays (169,011 SNPs with call rate>90%, mean call rate=99.87%). Unaffected canines were those with no detectable osteosarcoma while affected canines were those with osteosarcoma diagnosis confirmed by a licensed veterinarian.

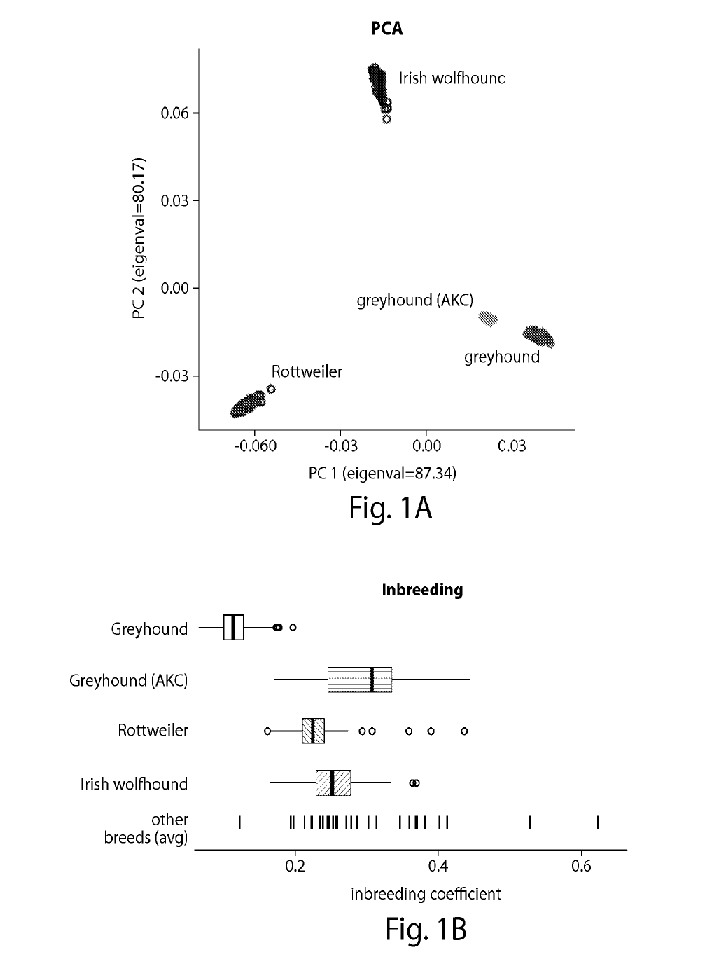

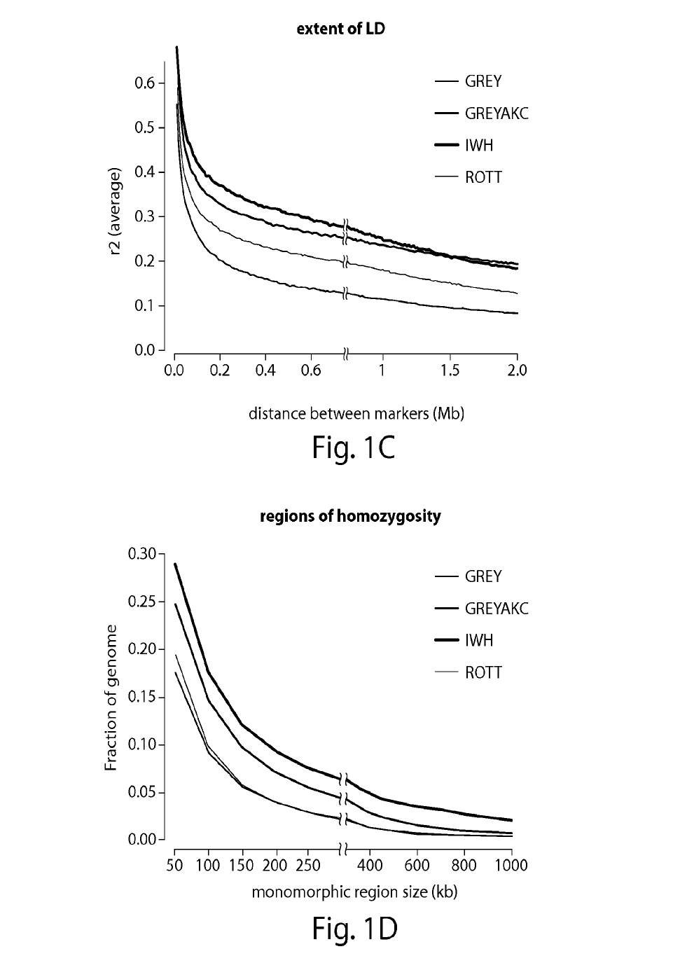

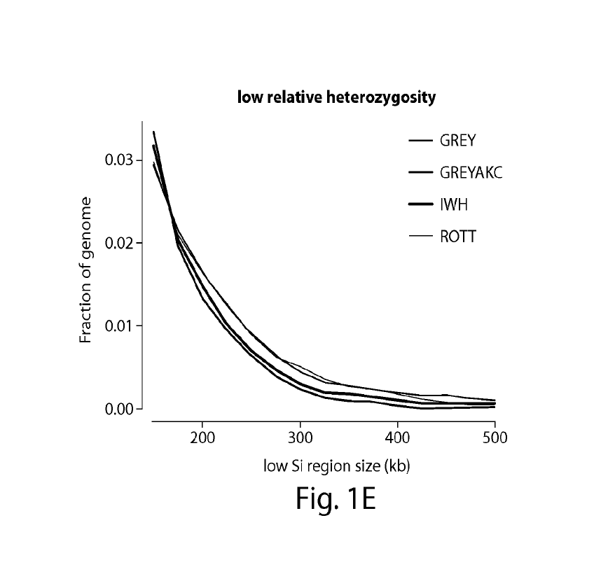

Each of the three breeds comprises a distinct population, with the AKC Grey clustering near their racing brethren (FIG. 1A). The Grey population was the least inbred, likely reflecting a large effective population size, (inbreeding coefficient 0=0.12+/−0.04), followed by the Rotts (θ=0.22+/−0.03), IWHs (0=0.24+/−0.04) and AKC Greys (0.30+/−0.07) (FIG. 1B). Fewer than 1% of sample pairs shared estimated genetic relatedness (GR)>0.25 (first cousins or closer) [ref. 10]. Linkage disequilibrium in all breeds was long, as compared to human populations, but varied markedly by breed, with average r² dropping below 0.25 at >105 kb in the Grey, 280kb in the Rott, and 945 kb in IWH (FIG. 1C).

In each breed a substantial portion of the genome was fixed (with minor allele frequency (MAF)<0.05): 5.6% in the Grey, 5.8% in the Rott and 12.1% in IWH (FIG. 1D). In addition, potential selected regions were identified as those with unusually relatively reduced variability (RRVs) relative to a reference panel of 28 dog breeds (Si), a method shown effective for mapping canine phenotypes, such as chondrodysplasia and skin wrinkling, that are restricted to a small number of breeds (Vaysse et al., Identification of genomic regions associated with phenotypic variation between dog breeds using selection mapping. PLoS genetics 7 (2011)). The 1% extreme tail of Si, measured in 150 kb sliding windows, totaled 2.9% (277 regions), 2.9% (344 regions) and 3.1% (387 regions) of the autosomal genome in the Grey, Rott and IWH respectively.

### GWAS Identified 33 Regions of Association

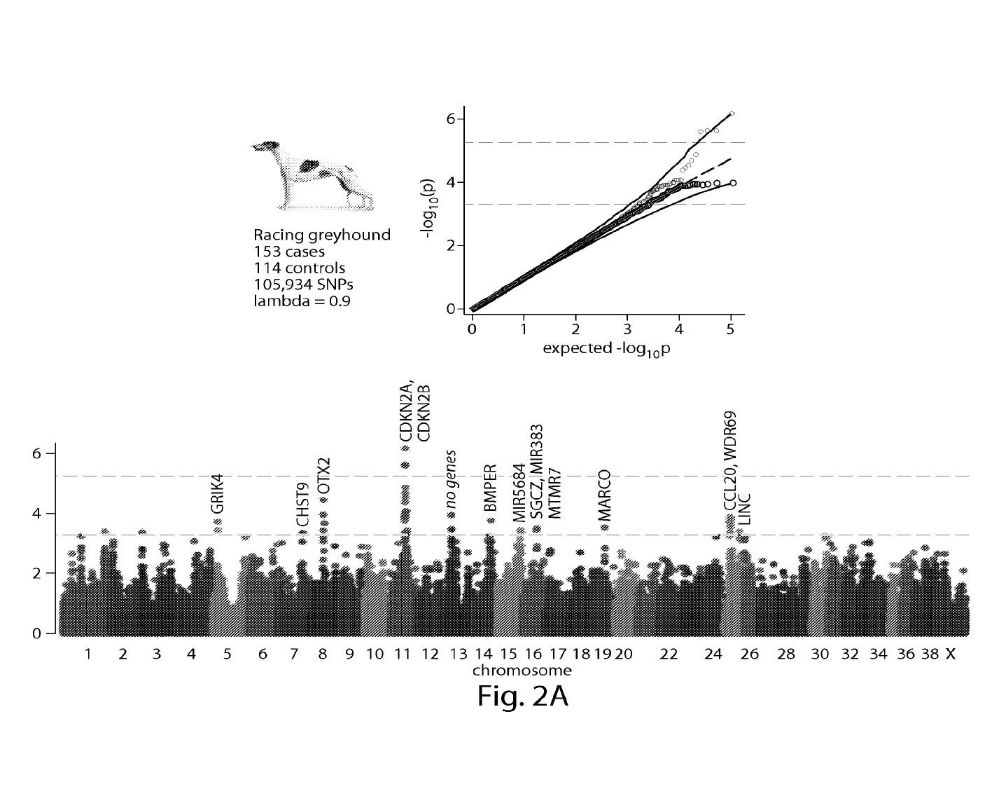

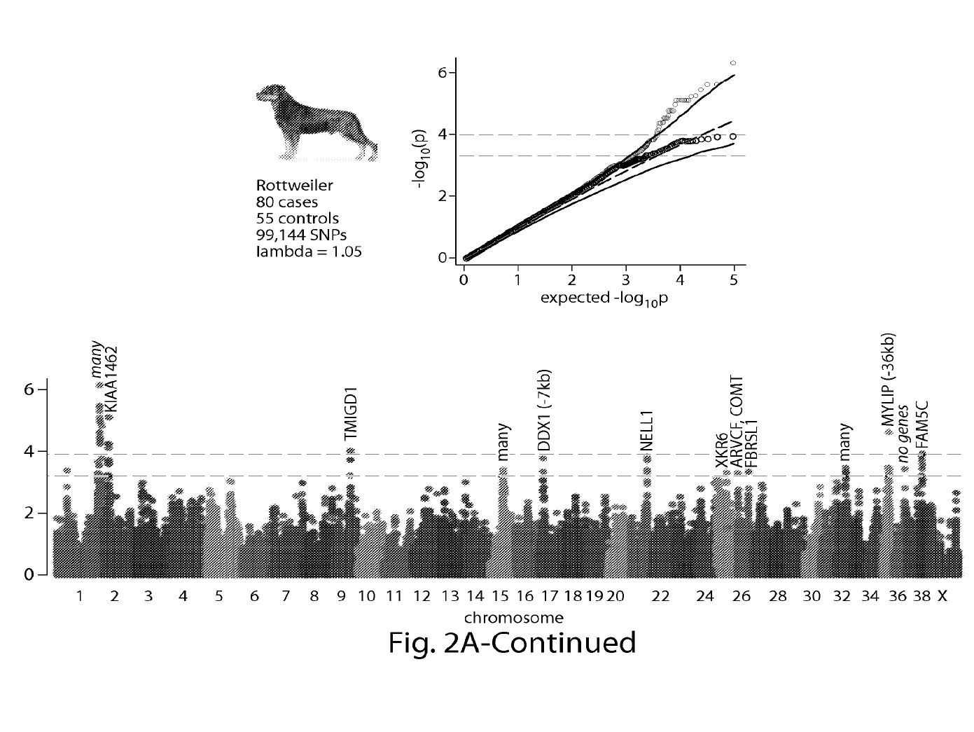

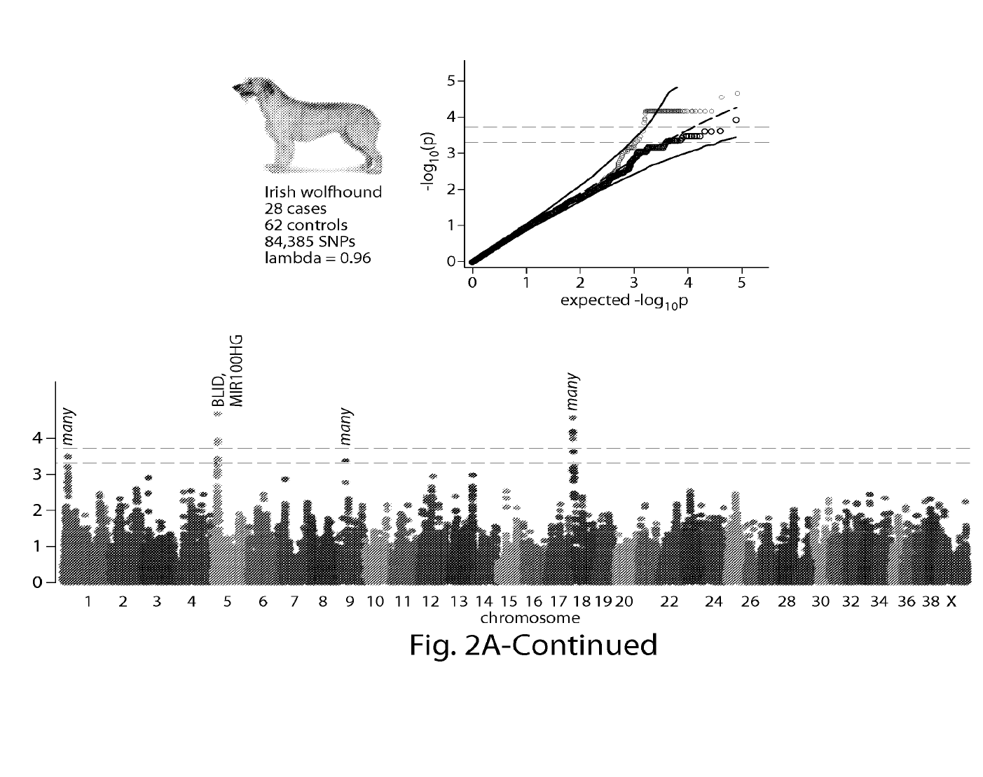

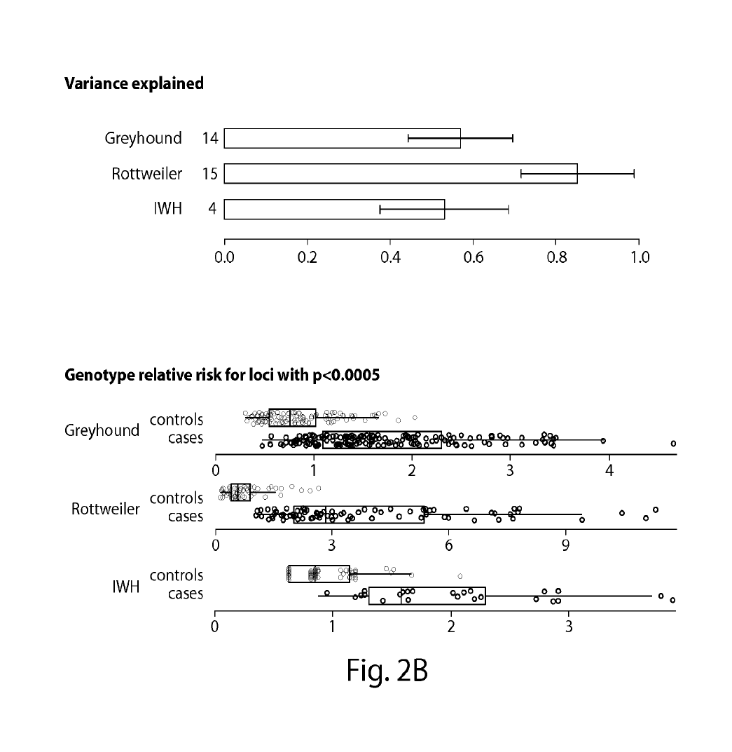

Association between germ-line variants with MAF>0.05 and osteosarcoma in each of the three breeds independently were tested, rigorously controlling for the complex population structure in breeds by: (1) excluding one dog from each matched phenotype pair with GR>0.25, preferentially retaining younger cases and older controls; and (2) controlling for cryptic relatedness using a mixed model approach with the top principle component as a covariate [ref. 11 and 12]. The final dataset included 267 Greys (153 A+114 U; 105,934 SNPs with MAF>0.05), 135 Rotts (80 A+55 U; 99,144 SNPs) and 141 IWH (76 A+65 U). After finding no significant associations in the full set of IWH, an age-stratified dataset was next focused on (28 A<6 years old and 62 U>6 years old, 84,385 SNPs). All identified SNPs either had a significant association (exceeding 95% confidence intervals defined empirically using 1000 random permutations; FIGS. 1A, C, E and FIGS. 2A and B) or suggestive association (p<0.0005). For each SNP, linkage disequilibrium patterns were used to define a region of association using the clumping methodology implemented in the software program PLINK [ref. 13] (Table 4). Finally, the proportion of phenotype variance explained by the associated loci was estimated using the software package GCTA (Genome-wide Complex Trait Analysis; Yang J, Lee S H, Goddard M E and Visscher P M. GCTA: a tool for Genome-wide Complex Trait Analysis. Am J Hum Genet. 2011 January 88(1): 76-82).

TABLE 4
SNPs and Associated Chromosomal Regions

| | | | | | | | | | | | |
|---|---|---|---|---|---|---|---|---|---|---|---|
| | | | | | | | | | | GRAIL | |
| | | | | | | | Size | | | candidate | |
| CHR | POS | GWAS P | F(Aff) | F(Un) | OR | Region | (kb) | # GENES | All Genes | genes | GRAIL p |

Greyhound

| | | | | | | | | | | | | |
|---|---|---|---|---|---|---|---|---|---|---|---|---|
| 1 | 11 | 44405676 | 6.41E−07* | 0.34 | 0.65 | 1.26 | chr11: 44392734 . . . 44414985 | 22 | 1 | CDKN2B-AS1 (50kb) | CDKN2B-AS1 | 0.0350 |
| 2 | 8 | 35448126 | 3.38E−05* | 0.12 | 0.03 | 1.36 | chr8: 35433142 . . . 35454649 | 22 | 1 | OTX2 (50kb) | OTX2 | 0.0014 |
| 3 | 13 | 14588716 | 1.00E−04 | 0.34 | 0.19 | 1.20 | chr13: 14549973 . . . 14645634 | 96 | 0 | | | |
| 4 | 25 | 21912859 | 1.27E−04 | 0.56 | 0.41 | 1.19 | chr25: 21831580 . . . 21921256 | 90 | 0 | | | |
| 5 | 14 | 49193217 | 1.64E−04 | 0.37 | 0.23 | 1.19 | chr14: 48831824 . . . 49203827 | 372 | 1 | BMPER | BNPER | 0.0014 |
| 6 | 5 | 16085937 | 1.82E−04 | 0.28 | 0.14 | 1.23 | chr5: 16071171 . . . 16152955 | 82 | 1 | GR1K4 | | |
| 7 | 19 | 34134931 | 2.79E−04 | 0.80 | 0.67 | 1.21 | chr19: 33963105 . . . 34145310 | 182 | 2 | EN1, MARCO | EN1 | 0.0093 |
| 8 | 16 | 43569044 | 3.00E−04 | 0.54 | 0.48 | 1.16 | chr16: 43665149 . . . 43737129 | 72 | 1 | MTNR7 | | |
| 9 | 15 | 63780452 | 3.43E−04 | 0.91 | 0.91 | 1.26 | chr15: 63767963 . . . 63800415 | 32 | 0 | none | | |
| 10 | 16 | 40896559 | 3.46E−04 | 0.97 | 0.89 | 1.36 | chr16: 40083517 . . . 41081510 | 198 | 1 | SGCZ | | |
| 11 | 25 | 43485109 | 3.77E−04 | 0.22 | 0.11 | 1.23 | chr25: 43476429 . . . 43528145 | 52 | 1 | CCL20 | | |
| 12 | 1 | 112990983 | 3.63E−04 | 0.02 | 0.69 | 1.21 | chr1: 112977233 . . . 113081800 | 105 | 5 | CD3EAP, ERCC1, ERCC2, FOSB, | | |
| | | | | | | | | | | PPP1R13L | | |
| 13 | 3 | 5564882 | 4.02E−04 | 0.79 | 0.68 | 1.20 | chr3: 5162056 . . . 6465753 | 1301 | 3 | FER, MAN2A1, PJA2 | | |
| 14 | 7 | 64672328 | 4.22E−04 | 0.57 | 0.43 | 1.16 | chr7: 64631053 . . . 64703475 | 72 | 1 | CHST9 | | |

Rottweller

| | | | | | | | | | | | | |
|---|---|---|---|---|---|---|---|---|---|---|---|---|
| 1 | 1 | 116524913 | 5.01E−07* | 0.71 | 0.39 | 1.32 | chr1: 115582915 . . . 116790630 | 1208 | 35 | ADCK4, AKT2, AXL, BLVRB, C19orf47, | DLL3 | 0.0476 |
| | | | | | | | | | | C19orf54, CNTD2, CYP2A7, CYP2B6, | | |
| | | | | | | | | | | CYP2S1, DLL3, EGLN2, FBL, FCGBP, | | |
| | | | | | | | | | | GMFG, HIPK4, HNRNPUL1, ITPKC, | | |
| | | | | | | | | | | LEUTX, LTBP4, MAP3K10, MED29, | | |
| | | | | | | | | | | NUMBL, PLD3, PLEKHG2, PSMC4, | | |
| | | | | | | | | | | RAB4B, SAMD4B, SERTAD1, SERTAD3, | | |
| | | | | | | | | | | SHKBP1, SNRPA, SPTBN4, SUPT5H, | | |
| | | | | | | | | | | TIMM50 | | |
| 2 | 2 | 19515571 | 5.78E−06* | 0.91 | 0.73 | 1.43 | chr2: 19212450 . . . 19542015 | 530 | 1 | KIAA1462 | KIAA1462 | 0.0057 |
| 3 | 1 | 122048812 | 1.11E−05* | 0.73 | 0.46 | 1.30 | chr1: 122033806 . . . 122051988 | 19 | 3 | C19orf40, CEP89, RHPN2 | | |
| 4 | 35 | 18338700 | 1.81E−05* | 0.51 | 0.29 | 1.28 | chr35: 18326079 . . . 18345318 | 19 | 1 | | | |
| 5 | 9 | 47659782 | 7.56E−05* | 0.53 | 0.28 | 1.28 | chr9: 47647012 . . . 47668054 | 21 | 2 | BLMH, TMIGD1 | | |
| 6 | 38 | 11714169 | 9.35E−05* | 0.49 | 0.26 | 1.24 | chr38: 11252518 . . . 11739329 | 407 | 1 | FAM5C | FAM5C | 0.0470 |
| 7 | 21 | 46283811 | 1.13E−04 | 0.94 | 0.70 | 1.37 | chr21: 46231985 . . . 46363479 | 131 | 1 | NELL1 | NELL1 | 0.0008 |
| 8 | 17 | 14472761 | 1.31E−04 | 0.18 | 0.01 | 1.39 | chr17: 14465884 . . . 14482152 | 15 | 1 | | | |
| 9 | 32 | 25147661 | 2.70E−04 | 0.95 | 0.01 | 1.35 | chr32: 25136302 . . . 25156153 | 20 | 1 | EMCN | EMCN | 0.0074 |
| 10 | 36 | 29651125 | 2.98E−04 | 0.56 | 0.42 | 1.22 | chr36: 29637804 . . . 29663408 | 26 | 0 | | | |
| 11 | 15 | 38987072 | 3.12E−04 | 0.82 | 0.59 | 1.27 | chr19: 37986345 . . . 39974762 | 1988 | 15 | AMDHD1, CCDC38, CDK17, ELK3, | | |
| | | | | | | | | | | FGD6, HAL, LTA4H, METAP2, NETAP2, NDUFA12, | | |
| | | | | | | | | | | NEDD1, NR2C1, NTN4, SNRPF, USP44, | | |
| | | | | | | | | | | VEZT | | |
| 12 | 1 | 29775073 | 3.31E−04 | 0.46 | 0.23 | 1.24 | chr1: 29405587 . . . 29914411 | 509 | 2 | EYA4, TCF21 | TCF21 | 0.0085 |
| 13 | 26 | 32385934 | 3.71E−04 | 0.91 | 0.76 | 1.35 | chr26: 32374093 . . . 32428449 | 54 | 3 | ARVDF, C22orf25, CONT | | |
| 14 | 25 | 29671618 | 3.94E−04 | 0.23 | 0.05 | 1.30 | chr25: 29658978 . . . 29767164 | 108 | 1 | NKR6 | | |
| 15 | 26 | 3537143 | 4.16E−04 | 0.48 | 0.30 | 1.26 | chr26: 3229343 . . . 3550075 | 21 | 1 | FBRSL1 | | |

Irish Wolfhound

| | | | | | | | | | | | | |
|---|---|---|---|---|---|---|---|---|---|---|---|---|
| 1 | 5 | 15264066 | 2.12E−05* | 0.45 | 0.16 | 1.40 | chr5: 14720254 . . . 15466603 | 746 | 1 | BLID | BLID | 0.0198 |
| 2 | 18 | 4937944 | 2.75E−05* | 0.56 | 0.21 | 1.36 | chr18: 4266743 . . . 5854451 | 1588 | 6 | C7orf72, COBL, DDC, FIGNL1, GRB10, | VWC2 | 0.0052 |
| | | | | | | | | | | IKZF1, VWC2, ZPBP | | |
| 3 | 1 | 17742179 | 3.21E−04 | 0.46 | 0.19 | 1.31 | chr1: 16765869 . . . 18150476 | 1382 | 7 | BCL2, KIAA1468, PHLPF1, PIGN, | TNFRSF11A, | 0.0635 |
| | | | | | | | | | | RNF152, TNFRSF11A, ZCCHC2 | BCL2 | |
| 4 | 9 | 19623231 | 4.28E−04 | 0.14 | 0.02 | 1.75 | chr9: 18896060 . . . 19633155 | 737 | 4 | ABCA5, KCNJ16, KCNJ2, MAP2K6 | | |

In each of the breeds, 20-40% of the phenotype variance was explained by the handful of loci with genome-wide significant associations (1 locus in Greys, 2 in IWHs, 6 loci in Rotts) [ref. 10]. Including all regions with p<0.0005 increased the phenotype variance explained to 57% in the Grey (14 loci), 53% in the IWH (4 loci) and 85% in the Rotts (15 loci). Surprisingly, none of the regions of association overlaps between the breeds, in contrast to the pattern observed for Mendelian canine traits [ref. 14], and meta-analysis of the three breeds also yielded no significant associations.

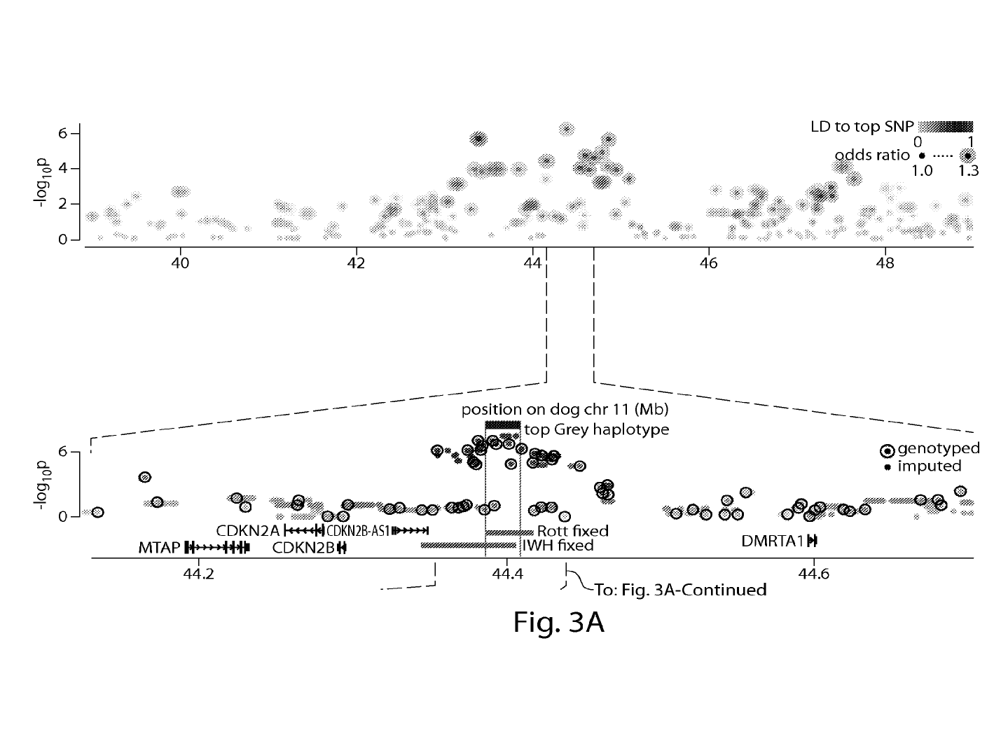

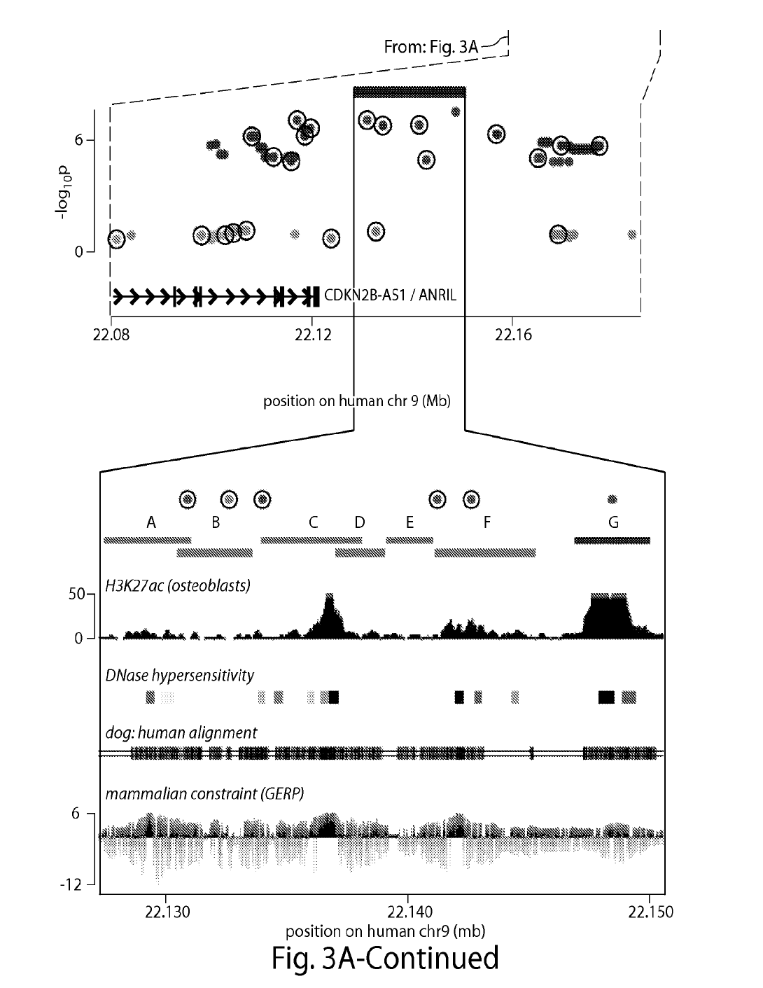

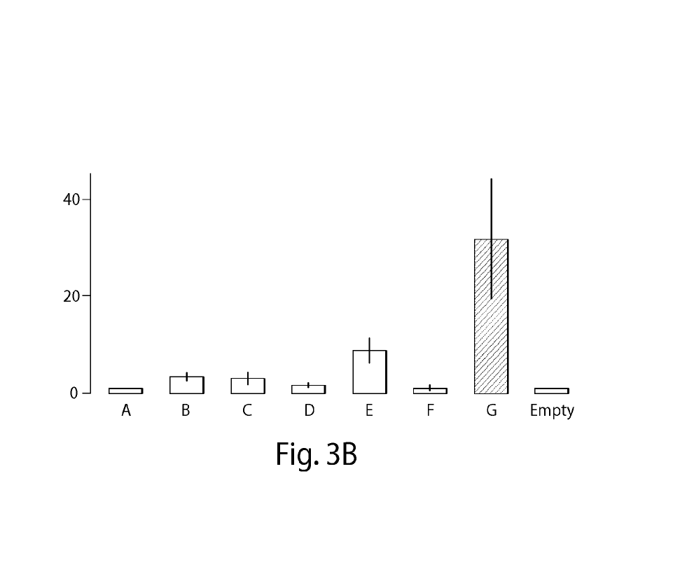

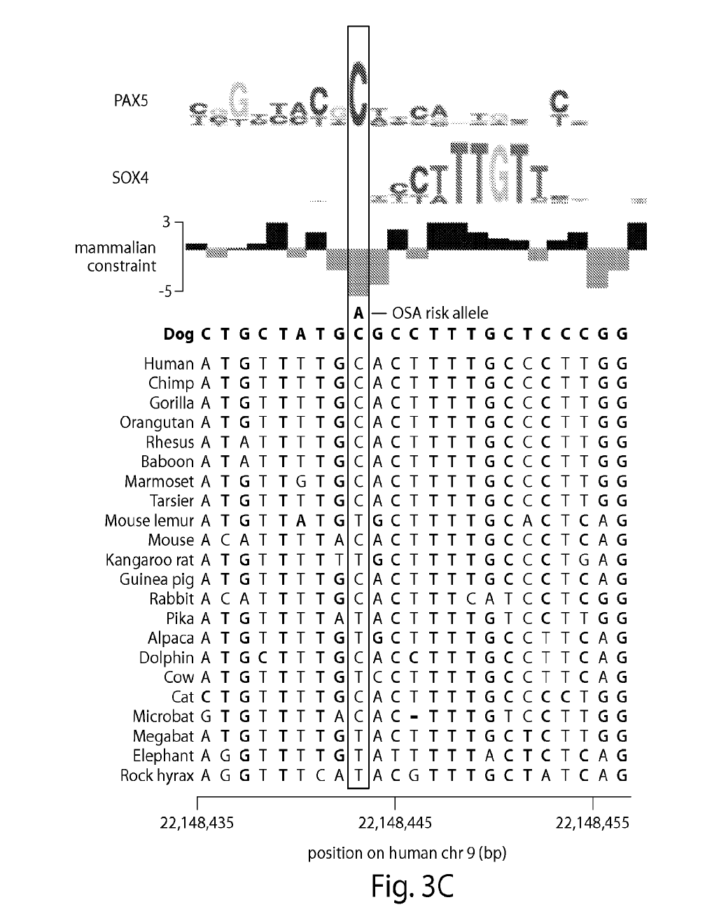

By examining fixed genomic regions one potential shared risk locus was identified: the risk allele tagging the top associated Grey locus is found at exceptionally high frequency in both the Rotts (97%) and IWH (95%), as compared to 51%+1-24% for 28 other dog breeds and 61% for the unaffected AKC Greys. This locus contains two well characterized tumor suppressors, CDKN2A (encodes p16^{IINTK4a} and p19^{ARF}) and CDKN2B (p15^{INK4b}), and the antisense non-coding gene CDKN2B-AS/ANRIL (FIG. 3A). The region of association in the Greys was narrowed to ˜111 kb upstream of the 5′ end of ANRIL by first sequencing chr11:43.0-48.9 Mb in 15 Greys (8 cases and 7 controls, 16,475 variants) and then genotyping 140 variants in 180 cases and 115 controls. Imputation yielded 1307 variants with MAF>0.01 (FIG. 3B). The top scoring variants encompass a 15 kb haplotype (chr11:44,390,633-44,406,002, 86% in the cases and 68% in the controls) positioned 10 kb downstream of ANRIL. Validation genotyping in Rotts (92A+67U), IWH (22A+30U) and 6 additional osteosarcoma affected breeds (23A+21U Great Danes, 16A+20U Great Pyrenees, 33A+35U golden retrievers, 9A+15U Labrador retrievers, 24A+22U Leonbergers and 13A+11U mastiffs) confirms the risk haplotype in this 15 kb region is essentially fixed in the Rotts (F_{A}=0.98,F_{U}=0.96) and IWH (F_{A}=0.95,F_{U}=0.92) and weakly associated with osteosarcoma in the Leonberger (F_{A}=0.81,F_{U}=0.64, p=0.06) and Great Pyrenees (F_{A}=0.78, F_{U}=0.65, p=0.22).

### Pathway Analysis of all Associated Regions

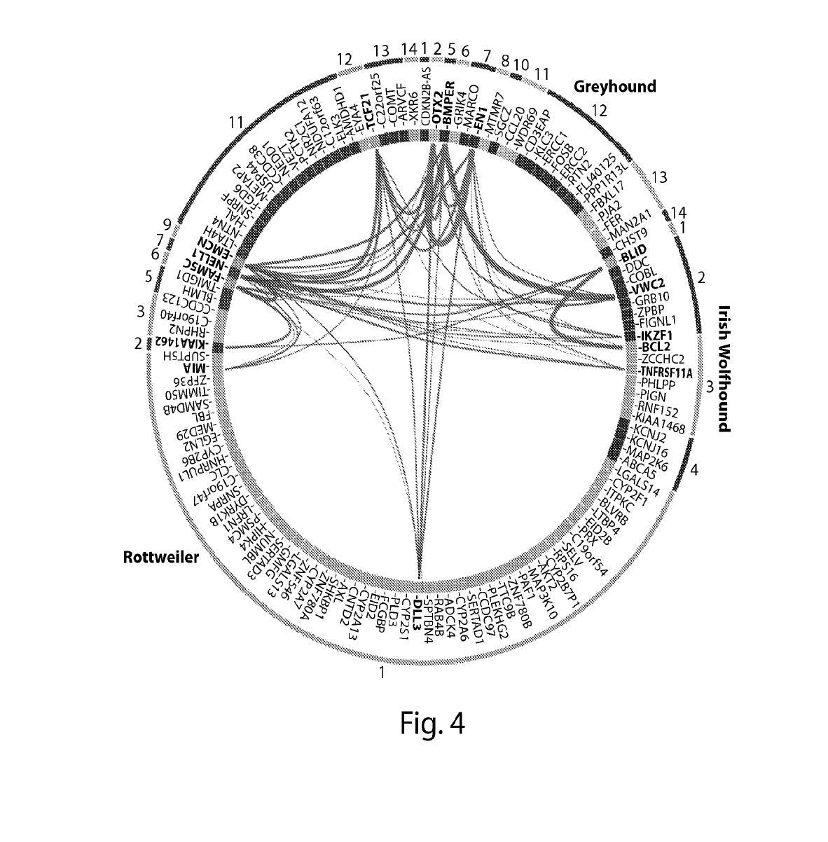

GRAIL (Gene Relationships Across Implicated Loci) was used to identify non-random connectivity between genes in associated loci described herein [ref. 18], finding enrichment for relevant descriptors including “bone” (13 loci), “differentiation” (13 loci), “development” (9 loci) and “notch” (7 loci). Notch signaling is critical to osteosarcoma invasion and metastasis [ref. 19]. In 12 of 26 genic loci, GRAIL identified highly connected candidate genes (p<0.05) with intriguing relevance to osteosarcoma (Table 4, FIG. 4). OTX2, the only gene in the second most associated Grey locus, encodes an oncogenic orthodenticle homeobox protein that directly activates cell cycle genes and inhibits differentiation in medulloblastomas [ref. 20]. GRAIL connected OTX2 with genes in 6 other risk loci (p<0.05): two negative regulators of osteoblast differentiation BMPER (Grey) and VWC2 (IWH)) [ref. 21]; EN1 (Grey), a modulator of osteoblast differentiation and proliferation (22); DLL3 (Rott), notch ligand implicated in human skeletal growth disorders [ref. 23]); TCF21 (Rott), a tumor suppressor that regulates mesenchymal-epithelial cell transitions; and EMCN (Rott), a mucin-like anti-adhesion membrane protein and hematopoietic stem cell marker [ref. 24]).

Osteoblast differentiation enhancer FAM5C (Rott) [ref. 25] is connected by GRAIL to NELL1 (Rott), a regulator of osteoblast differentiation and ossification; TNFRSF11A (IWH), an essential mediator of osteoclast development; and the pro-apoptotic gene BLID (IWH).

GRAIL was also used to analyze regions in which the racing and osteosarcoma unaffected AKC Greys differed, defining the most differentiated SNPs using emmax (p<1×10⁻⁹) and then clumping them into 68 LD defined regions in PLINK (median size 387 kb, 5.1% of genome). GRAIL analysis of the results detected strong interconnectivity between a number of genes involved in “RNA” related cellular mechanisms, including small nucleolar RNAs in 6 distinct genomic regions (SNORA79, SNORA39, SNORA59A, SNORA6, SNORD87, SNORA62 and SNORD17, SNHG6) and genes related to hormones, catenin complexes and telomerase. Pathway analysis using INRICH (Lee et al. INRICH: Interval-based Enrichment Analysis for Genome Wide Association Studies. Bioinformatics. 2012 Jul. 1; 28(13):1797-9.) on the same set of regions yielded a single significant gene set enrichment after permutation: genes with the MIR-512-5P binding cis regulatory motif GCTGAGT (p=7e-05, pcorr=0.03, regulating genes DDX6, CTNNB1, CHD9, XKR6, STC1, NUDT18, ERP29, GNAZ, GRK6).

### Fixed and Selected Loci in Breeds Contribute to Disease Risk

Fixed regions longer than 250 kb comprised a large proportion of the genome in each breed (Grey: 2.8%; Rott: 12.9%; IWH: 7.6%) encompassing genes linked to bone development and osteosarcoma, including RB1 (IWH), FOS (Rott), RUNX2 (Rott), CCNB1 (IWH), COL11A2 (Grey) and POSTN (IWH and Grey) [ref. 27]. In total 72.2 Mb (3.3%) of the genome were fixed in all three breeds (N=492, mean size=147 kb, 72.2 Mb total). These shared regions were enriched for microRNAs associated with pathogenesis and progression of osteosarcoma (p=0.017, pcorr=0.042, MIR150, MIR335, MIR340, MIR663, MIR650) [ref. 28]. When examined, the potentially selected RRVs INRICH enrichment (pcorr=0.035) was detected for putative “driver” genes of human osteosarcoma (WASF3; KIAA1279; AIFM2; CLCC1) [ref. 29].

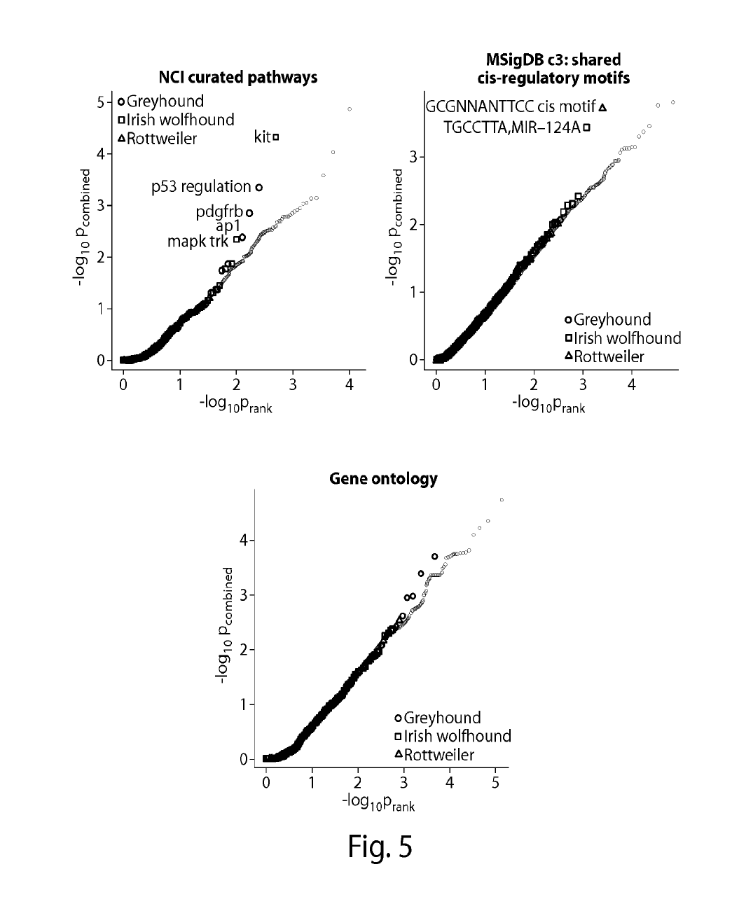

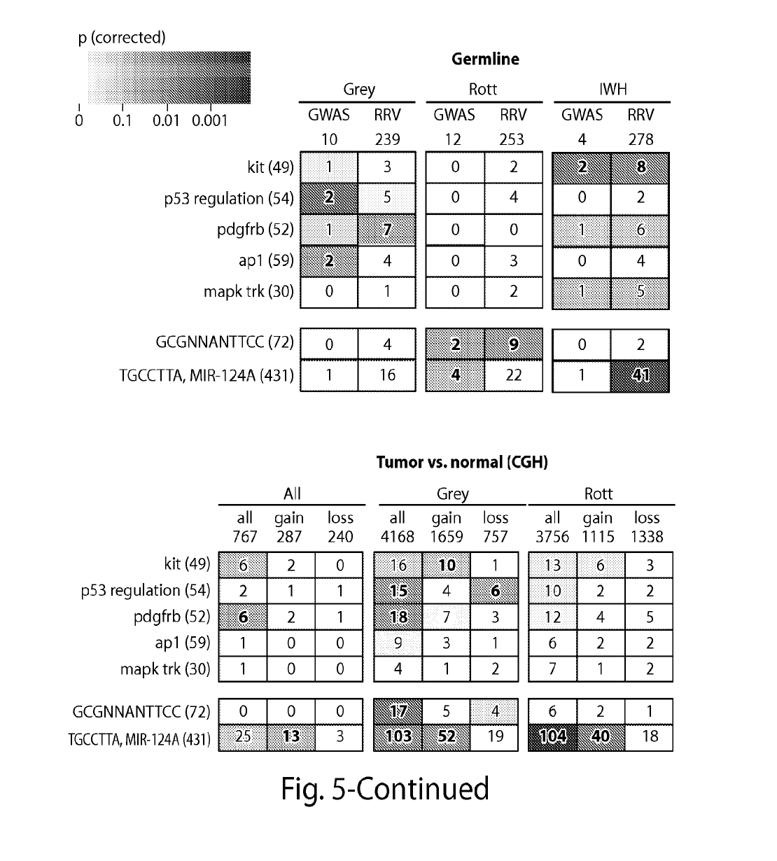

To formally test whether the GWAS loci and RRVs are enriched for the same pathways, the INRICH results from the GWAS were combined with INRICH results for the RRs results from each breed using the Fisher method. The same analysis was performed with RRVs from 28 other breeds as a control (FIG. 5). It was found that, while the vast majority of gene sets in the studied breeds show no increase in significance, a small number were markedly inflated, including the kit, p53 and pdgfrb pathways from the NCI Nature curated cancer pathways and two MSigDB genes based on shared promoter region transcription start site motifs—targets of MIR-124A (TGCCTTA) and a highly conserved motif with no known transcription factor match [ref. 30].

### GWAS Pathways Enriched for Somatic Mutations in Osteosarcoma Tumors

Somatic tumor DNA was compared to blood-derived germ-line DNA in a subset of 7 affected Greys and 7 affected Rotts using array-based comparative genomic hybridization (aCGH) with a new, dense 180,000-feature Agilent canine CGH microarray (˜13 kb resolution). It was found that 99.7% of autosomal loci (162,858/162,337) had either a gain or loss in at least one dog (log₂ tumor:reference signal intensity ratio>+/−0.2). On average, 49.6%+/−11.0% of the loci were altered in each Grey tumor and 56.1%+/−10.8% in each Rott tumor. Particular probes were enriched for changes; the fraction of probes altered in all 7 Rotts (N=8087, 4.95%), all 7 Greys (N=8781, 5.35%) or all 14 dogs (N=1603, 0.98%) was much higher than expected by random chance (p_{binomial}=2.71%, 1.3% and 0.04% respectively). Putative human osteosarcoma driver genes were among those with universal CGH loss in Greys (ARHGAP22, ARID5B, RCBTB1), Rotts (LHFP), and both breeds (AIFM2, TSC22D1) [ref. 29]. Comparing the genes affected by these high frequency alterations to genes altered in human osteosarcoma cell lines highlight the similarities between dog and human osteosarcoma.

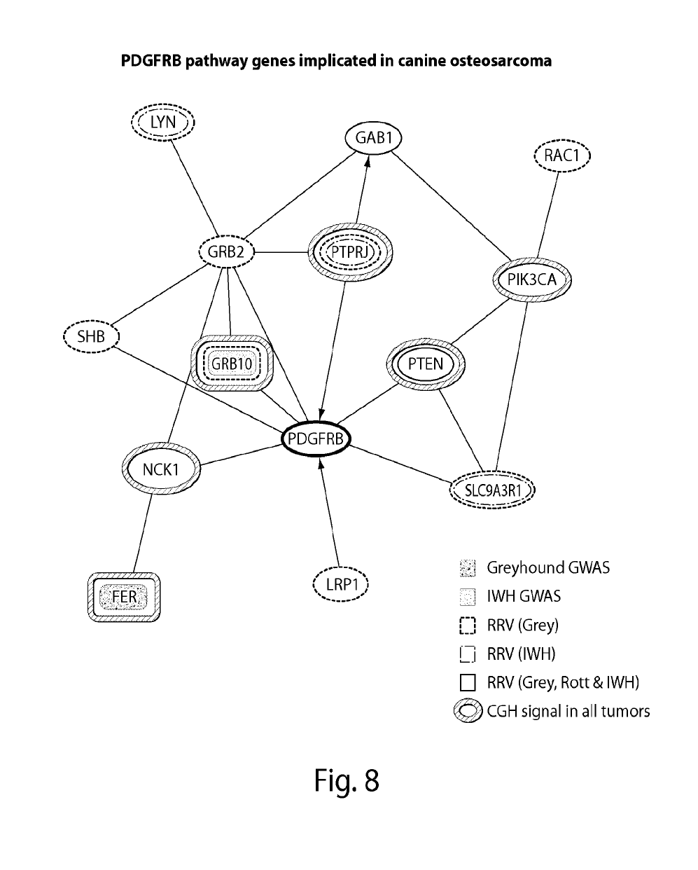

It was then tested whether the 7 gene sets identified by combining GWAS and RRV pathways (FIG. 5) also were enriched for somatic changes in tumors. It was found that nearly every gene set pathway showed strong enrichment in one or both breeds. The set of genes with the MIR-124A cis-regulatory motif, discovered in IWH germ-line data, showed strong signals of enrichment in the CGH altered loci from both Grey and Rott. In human cancer cells, epigenetic loss of MIR-124A is linked to activation of CDK6 and phosphorylation of Rb1 [ref. 32]. The PDGFR-beta signaling pathway was enriched for genes with CGH alteration in all 14 tumors tested (FIG. 8). The p53 regulation pathway, identified in the Grey germ-line data, also showed significant enrichment in Grey CGH loci (10 genes), but is just weakly enriched in Rott, potentially suggesting breed specific pathways that may underlie differences in disease etiology. Curiously, the GCGNNANTTCC motif pathway, detected in the Rott GWAS and RRV regions, showed CGH enrichment only in the Grey.

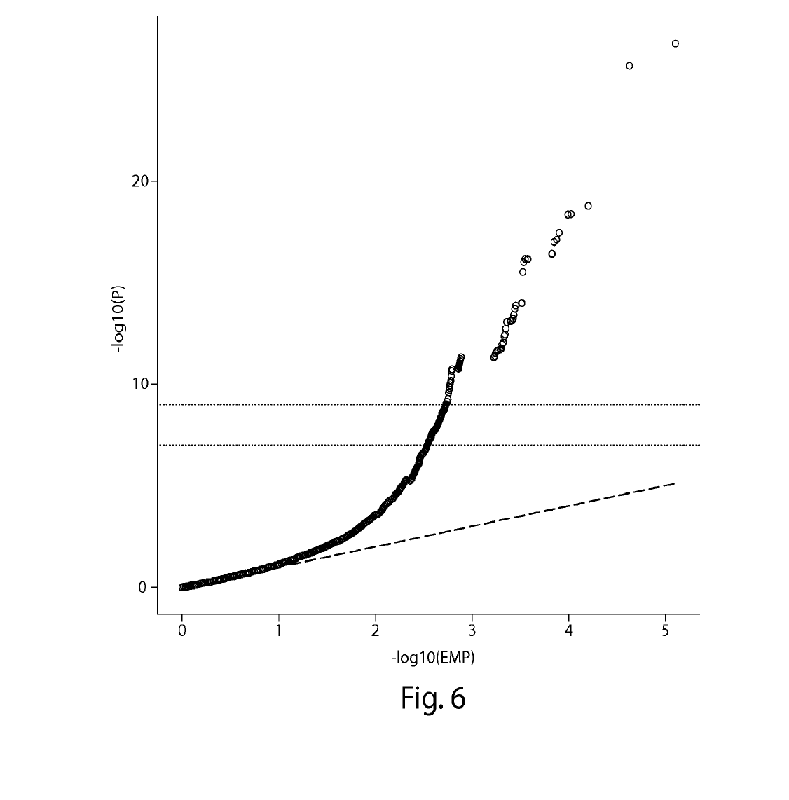

Additionally, an allele frequency comparison between the osteosarcoma-prone racing greyhounds and AKC greyhounds, which rarely get osteosarcoma identified candidate germline osteosarcoma risk variants (FIG. 6).

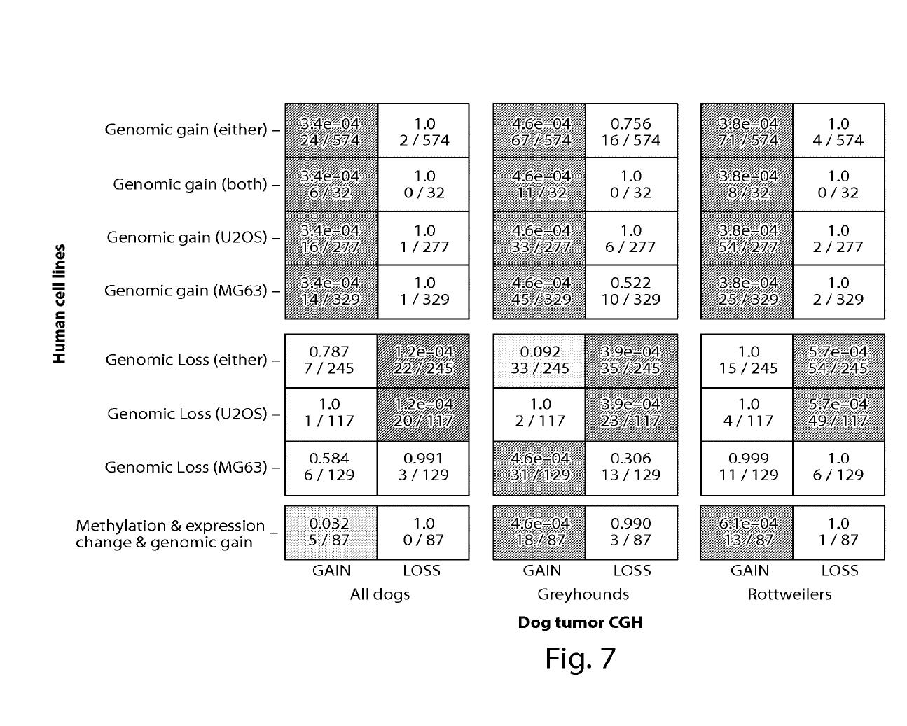

It was also found that there was highly significant overlap in the set of genes altered in canine osteosarcoma tumors and two human osteosarcoma cell lines (FIG. 7).

The correlations described in this example were confirmed in a second study involving a larger sample set.

### DISCUSSION

Osteosarcoma is an aggressive tumor of the bone that often metastasizes to the lung. Advances in chemotherapy have increased survival to about 60-70% but patients who present with pulmonary metastases, relapse or don't respond to chemotherapy continue to have a very poor prognosis. Increased understanding of disease etiology could improve therapy by subgrouping patients for treatment based on the underlying biology and also by suggesting mechanisms of tumor development that could be targeted. This is the first GWAS of osteosarcoma reported for any species.

Osteosarcoma in dogs is, both clinically and molecularly, remarkably similar to its human counterpart, but particularly high rates of osteosarcoma occur in some breeds. Here, just a few hundred dogs and ˜100,000 markers were used to explain the majority of phenotype variance within each breed. It was discovered that canine osteosarcoma has a complex genetic architecture; with up to 15 loci associated within a breed, far more than observed in other GWAS mapped canine diseases published thus far. Through comprehensive analysis of inherited genetic variation in these breeds combined with somatic alterations in osteosarcoma tumors a number of genes were identified that affect bone growth and differentiation as well as pathways for transformation and metastasis. The study herein confirms that osteosarcoma is heterogeneous in dogs, but highlights that among all risk factors identified some, e.g., CFA 11 (chr11:44392734-44414985), may be important in most of the affected individuals.

No apparent sharing of GWAS loci was identified between breeds, despite relatively recent shared genetic ancestry. Part of the explanation for this might be that while a large number of genes for osteosarcoma are present in the dog population as a whole, only a few make it into each breed. Through random chance each breed may inherit a different set of genetic risk factors resulting in mostly breed specific risk factors. As a few key risk factors become common in each breed they may then be sufficient to drive the disease development, suggesting that key pathways receive a substantial number of hits within a breed. This could allow dissection of functional pathways by examining different breeds.

Selection may further contribute to the enrichment of disease risk factors within breeds as osteosarcoma tends to affects large dogs. In humans, the tumor most commonly arises in conjunction with the adolescence growth spurt. This suggests that pathways for tissue growth and in particular osteogenesis may be involved in tumor development and this was also supported by the study herein. In general, dog breeds have been generated by breeding towards desirable traits and away from undesirable characteristics within a more or less closed gene pool. This artificial selection has resulted in fixed regions within a breed where all individuals carry the same haplotype. It is possible that selection for size and rapid growth in some breeds have resulted in the fixation of alleles that increase not only bone growth but also the risk of osteosarcoma development. This is evidenced by the top locus described herein, the candidate region on CFA11 identified by association in greyhounds. On closer examination, it was noted that the greyhound risk haplotype occurred in almost all Rottweilers and Irish Wolfhounds in the study, regardless of whether they were affected or free of disease, but not in AKC Greyhounds—a breed not predisposed to osteosarcoma.

The shared risk haplotype on CFA11 (chr11:44392734-44414985) encompasses sequence downstream of ANRIL, a long non-coding RNA regulating the expression of the CDKN2A/B locus which encodes tumor suppressors p16^{INK4a}, p19^{ARF} and p15^{INK4b} H3K27Ac histone marks in an osteosarcoma cell line indicate the presence of an active enhancer element in the haplotype sequence suggesting that SNPs in this region may influence expression of ANRIL in blood (Cunnington et al 2010). Human osteosarcomas display deletion of the orthologous 9p21 locus in 5-21% of cases (reviewed in Martin et al 2012). Correspondingly, mice where the CDKN2/A region has been deleted are known to be tumor-prone (Serrano et al 1996), and more recently it was shown that mice that have the CDKN2A/B locus intact but where 70 kb encompassing part of ANRIL has been deleted show increased risk of developing sarcomas (Visel et al). Furthermore, absence of p16INK4a expression has been correlated with decreased survival in pediatric osteosarcoma patients (Maitra et al 2001). Taking these observations together, we hypothesize that the risk haplotype carries enhancer elements in the ANRIL region, which result in increased expression of ANRIL and thereby cause the down regulation of the CDKN2A/B genes resulting in susceptibility to the initial steps of tumor development. Interestingly, another cancer GWAS in dogs also indicates association with this CFA11 region. Shearin et al report association of a haplotype spanning the MTAP gene and part of CDKN2A with risk of histiocytic sarcoma in Bernese Mountain Dogs (Shearin et al 2012).

### Example 2

2.5 Mb around the greyhound GWAS peak on chromosome 11 (chr11:44392734-44414985) was targeted for dense sequencing (15 dogs) and finemapping (180 cases and 115 controls). Imputation and association testing of sequenced variants narrowed the peak of association in greyhounds dramatically to a 20 kb risk haplotype (chr11:44390000-44410000), telomeric of the genes CDKN2A and CDKN2B, that is nearly fixed in both the rottweilers (98% in cases and 96% in controls) and Irish wolfhounds (95% in cases and 92% in controls). The top haplotype (vertical solid lines) mapped to a locus downstream of the non-coding gene ANRIL on human chromosome 9 (hg19). Potential markers of function in the region included H3K27 acetylation in osteoblasts and DNAase hypersensitivity clusters (assayed from 125 cell types), most notably in regions that align between the dog and human genomes in a Multiz alignment of 46 species and are constrained across mammals as measured by Genomic Evolutionary Rate Profiling (GERP) [refs. ENCODE Nature 2012, Davydov PLoS Comput Biol 2010, Meyer Nucleic Acids Res. 2012 Nov. 15, Rosenbloom Nucleic Acids Res. 2012].

The top haplotype genomic region was tiled with luciferase probes to assay function of seven sections (A-G, FIG. 3A) of the genomic region in osteosarcoma cell lines (see methods below). Of the seven non-control luciferase assays, four sections of the genomic region (B, C, E and G) showed a significant increase in luciferase activity compared to empty vector (FIG. 3B). Construct G showed the strongest increase with a ˜32-fold increase in activity, suggesting the presence of a strong enhancer within the genomic region encompassed by G. Fragment G was found to contain one of the top SNPs (BICF2P133066, chr11:44405676) which has a constrained reference allele C corresponding to a predicted transcription factor binding site, while the risk allele, A, is not found among 29 mammals or the wolf (FIG. 3C).

### Method of Enhancer Expression in U2OS Cells

Human chromosome 9 genomic region fragments A to G (FIG. 3A) were PCR amplified from human gDNA and placed in front of minimal promoter driven luciferase reporter gene (pGL4.26, Promega). Human osteosarcoma U2OS cells were seeded in 96 well plates (25 000 cells/well) and grown for 24-26 h before transfection. Each well was transfected with 0.1 ug reporter construct and 0.01 ug renilla luciferase driven by CMV promoter to control for cell density, using 0.4 ul/well FuGENE (Promega) according to the manufacturer's instructions. 24 h after transfection, activity of both luciferases was measured sequentially using the Dual-Glo Luciferase System (Promega) using a luminometer. Four independent experiments were performed, each with eight technical replicates of every construct.

### Example 3

Other genomic variants, such as SNPs and chromosomal regions, within or near CFA11(chr11:44392734-44414985) were found to be associated with osteosarcoma. These variants are listed in Table 5. The chr11:44405676 variant was identified as the top variant based on functional data. The correlations described in this example were confirmed in a second study involving a larger sample set.

TABLE 5
Additional variants associated with osteosarcoma

| VARIANT | RISK ALLELE | P value |
|---|---|---|
| chr11: 44390632 | T | 9.05E−08 |
| chr11: 44391818 | A | 0.09107 |
| chr11: 44392971 | G | 1.77E−07 |
| chr11: 44397317 | C | 3.20E−08 |
| chr11: 44399002 | T | 3.20E−08 |
| chr11: 44401361 . . . 44401371 | T | 3.20E−08 |
| chr11: 44402703 | C | 1.26E−05 |
| chr11: 44405676 | A | 3.20E−08 |

### Example 4

### Leonberger Osteosarcoma GWAs

### Methods

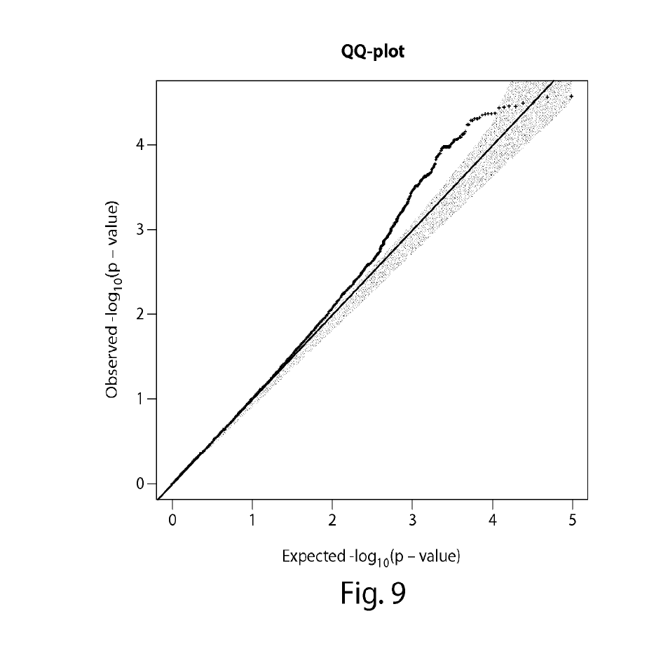

280 US leonberger dogs and 71 European (EU) leonberger dogs were included in this study. There were 138 cases and 213 controls total (182 US cases, 98 US controls, 40 EU cases, and 31 EU controls). Outliers, duplicates and uncertain phenotypes were removed. The call rate for SNPs and inds was >95%. The MAF>5%. The Hardy-Weinberg p>1E-6 in controls (FIG. 9).

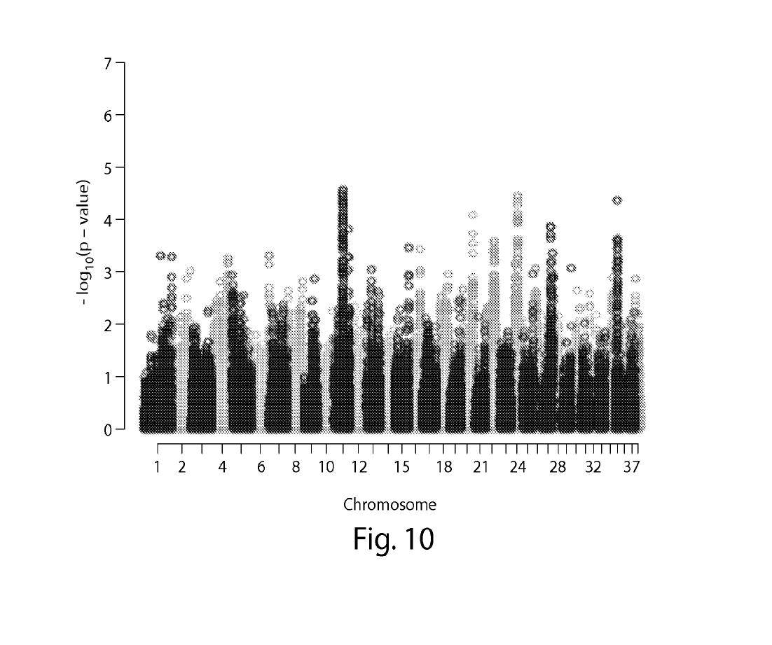

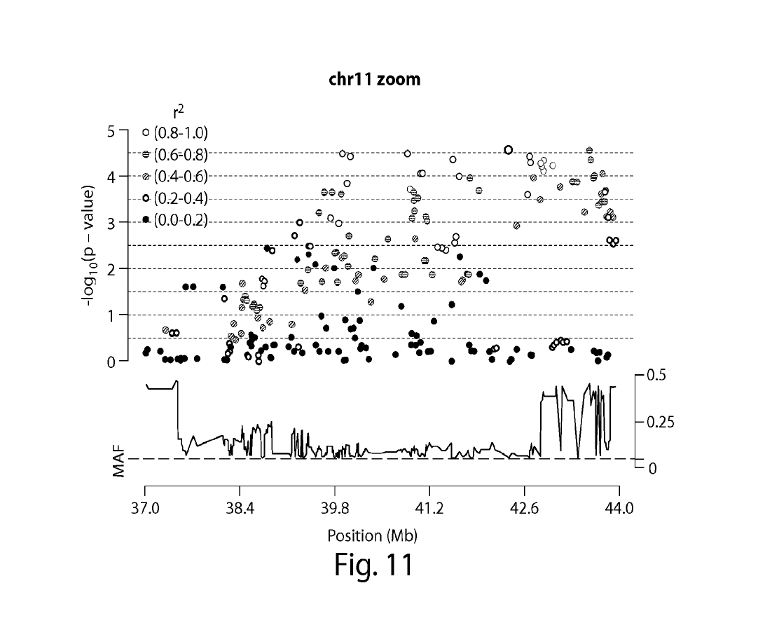

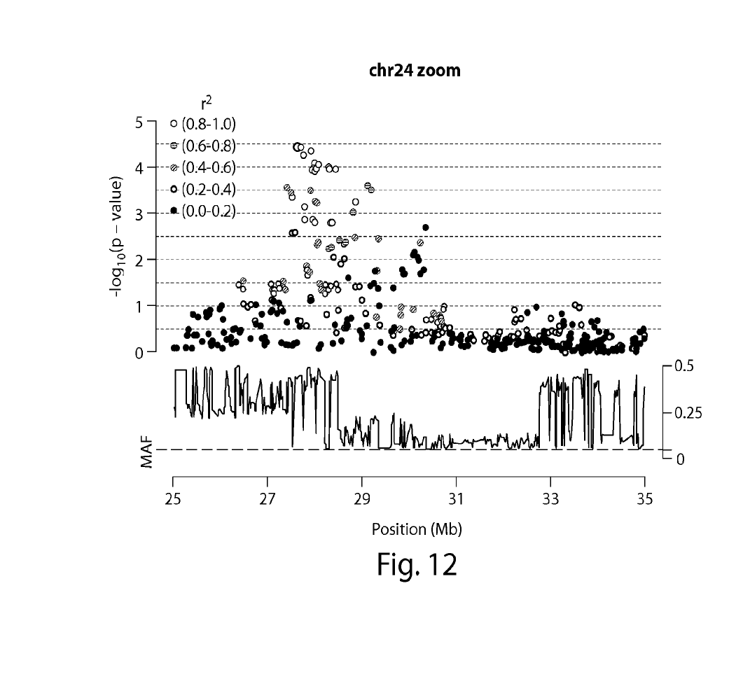

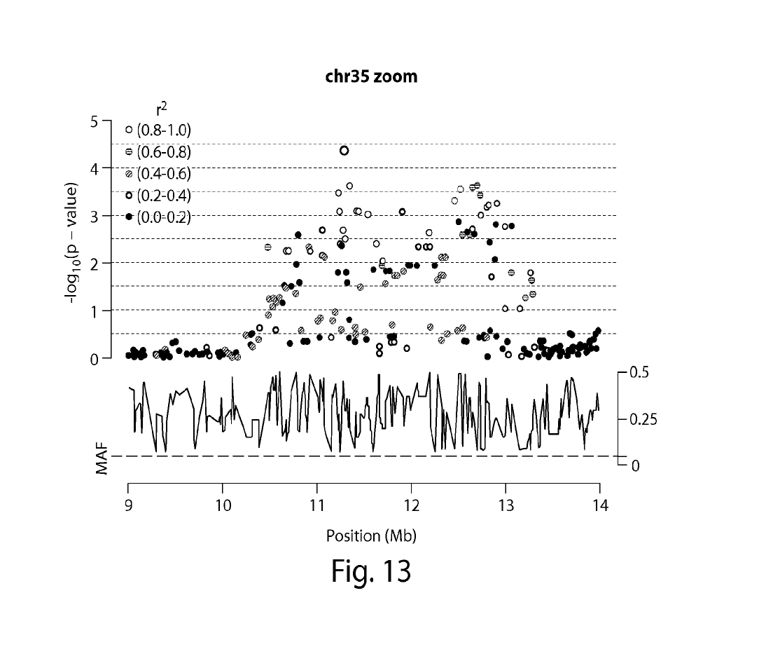

Regions on chromosomes 11, 24, and 35 had a large number of significant SNPs (FIG. 10), indicating regions of association with osteosarcoma. The top regions of association on chromosomes 11, 24, and 35 were determined based on the location of the top 100 SNPS. These regions are shown in Table 6 (coordinates are CanFam3 coordinates, see UCSC Genome Browser) and in FIGS. 11-13.

TABLE 6
Regions of association with osteosarcoma in Leonberger dogs

| Chromosome | Coordinates (canfam3) |
|---|---|
| chr11 | 39643190-45990018 |
| chr24 | 27409719-29194396 |
| chr35 | 11233053-12732906 |

Larger regions were determined based on sweeps of the chromosomal regions. These larger regions are shown in Table 7.

TABLE 7
Regions of associate with osteosarcoma in Leonberger dogs

| Chromosome | Coordinates (canfam3) |
|---|---|
| chr11 | 37,000,000-44,000,000 |
| chr24 | 27,000,000-33,000,000 |
| chr35 | 10,000,000-14,000,000 |

### REFERENCES

1. N. Tang, W. X. Song, J. Luo, R. C. Haydon, T. C. He, Osteosarcoma development and stem cell differentiation. Clinical orthopaedics and related research 466, 2114 (September, 2008).
2. U. Basu-Roy, C. Basilico, A. Mansukhani, Perspectives on cancer stem cells in osteosarcoma. Cancer letters, (May 29, 2012).
3. S. D. Berman et al., Metastatic osteosarcoma induced by inactivation of Rb and p53 in the osteoblast lineage. Proceedings of the National Academy of Sciences of the United States of America 105, 11851 (Aug. 19, 2008).
4. C. R. Walkley et al., Conditional mouse osteosarcoma, dependent on p53 loss and potentiated by loss of Rb, mimics the human disease. Genes & development 22, 1662 (Jun.
15, 2008).
5. M. Paoloni et al., Canine tumor cross-species genomics uncovers targets linked to osteosarcoma progression. BMC genomics 10, 625 (2009).
6. M. Slatter. (2000), vol. 2012.
7. S. R. Urfer, Lifespan and Causes of Death in the Irish Wolfhound: Medical, Genetical and Ethical Aspects. (Suedwestdeutscher Verlag fuer Hochschulschriften, 2009), pp. 160.
8. L. K. Lord, J. E. Yaissle, L. Marin, C. G. Couto, Results of a web-based health survey of retired racing Greyhounds. Journal of veterinary internal medicine/American College of Veterinary Internal Medicine 21, 1243 (Nov.-December, 2007).
9. G. Ru, B. Terracini, L. T. Glickman, Host related risk factors for canine osteosarcoma. Vet J 156, 31 (July, 1998).
10. J. Yang, S. H. Lee, M. E. Goddard, P. M. Visscher, GCTA: a tool for genome-wide complex trait analysis. American journal of human genetics 88, 76 (Jan. 7, 2011).
11. A. L. Price, N. A. Zaitlen, D. Reich, N. Patterson, New approaches to population stratification in genome-wide association studies. Nature reviews 11, 459 (July, 2010).
12. H. M. Kang et al., Variance component model to account for sample structure in genome-wide association studies. Nature genetics 42, 348 (April, 2010).
13. S. Purcell et al., PLINK: a tool set for whole-genome association and population-based linkage analyses. American journal of human genetics 81, 559 (September, 2007).
14. E. K. Karlsson et al., Efficient mapping of mendelian traits in dogs through genome-wide association. Nat Genet 39, 1321 (November, 2007).
15. E. Pasmant, A. Sabbagh, M. Vidaud, I. Bieche, ANRIL, a long, noncoding RNA, is an unexpected major hotspot in GWAS. FASEB journal: official publication of the Federation of American Societies for Experimental Biology 25, 444 (February, 2011).
16. K. L. Yap et al., Molecular interplay of the noncoding RNA ANRIL and methylated histone H3 lysine 27 by polycomb CBX7 in transcriptional silencing of INK4a. Molecular cell 38, 662 (Jun. 11, 2010).
17. Y. Kotake et al., Long non-coding RNA ANRIL is required for the PRC2 recruitment to and silencing of p15(INK4B) tumor suppressor gene. Oncogene 30, 1956 (Apr. 21, 2011).
18. S. Raychaudhuri et al., Identifying relationships among genomic disease regions: predicting genes at pathogenic SNP associations and rare deletions. PLoS genetics 5, e1000534 (June, 2009).
19. P. Zhang, Y. Yang, P. A. Zweidler-McKay, D. P. Hughes, Critical role of notch signaling in osteosarcoma invasion and metastasis. Clinical cancer research: an official journal of the American Association for Cancer Research 14, 2962 (May 15, 2008).
20. J. Bunt et al., OTX2 directly activates cell cycle genes and inhibits differentiation in medulloblastoma cells. International journal of cancer. Journal international du cancer 131, E21 (Jul. 15, 2012).
21. N. Koike et al., Brorin, a novel secreted bone morphogenetic protein antagonist, promotes neurogenesis in mouse neural precursor cells. The Journal of biological chemistry 282, 15843 (May 25, 2007).
22. R. A. Deckelbaum, A. Majithia, T. Booker, J. E. Henderson, C. A. Loomis, The homeoprotein engrailed 1 has pleiotropic functions in calvarial intramembranous bone formation and remodeling. Development 133, 63 (January, 2006).
23. M. P. Bulman et al., Mutations in the human delta homologue, DLL3, cause axial skeletal defects in spondylocostal dysostosis. Nat Genet 24, 438 (April, 2000).
24. A. Matsubara et al., Endomucin, a CD34-like sialomucin, marks hematopoietic stem cells throughout development. The Journal of experimental medicine 202, 1483 (Dec. 5, 2005).
25. K. Tanaka et al., FAMSC is a soluble osteoblast differentiation factor linking muscle to bone. Biochemical and biophysical research communications 418, 134 (Feb. 3, 2012).
26. G. A. Calin et al., Human microRNA genes are frequently located at fragile sites and genomic regions involved in cancers. Proceedings of the National Academy of Sciences of the United States of America 101, 2999 (Mar. 2, 2004).
27. T. M. Schroeder, R. A. Kahler, X. Li, J. J. Westendorf, Histone deacetylase 3 interacts with runx2 to repress the osteocalcin promoter and regulate osteoblast differentiation. The Journal of biological chemistry 279, 41998 (Oct. 1, 2004).
28. K. B. Jones et al., miRNA signatures associate with pathogenesis and progression of osteosarcoma. Cancer research 72, 1865 (Apr. 1, 2012).
29. M. L. Kuijjer et al., Identification of osteosarcoma driver genes by integrative analysis of copy number and gene expression data. Genes, chromosomes & cancer 51, 696 (July, 2012).
30. C. F. Schaefer et al., PID: the Pathway Interaction Database. Nucleic acids research 37, D674 (January, 2009).
31. A. Y. Angstadt et al., Characterization of canine osteosarcoma by array comparative genomic hybridization and R T-qPCR: signatures of genomic imbalance in canine osteosarcoma parallel the human counterpart. Genes, chromosomes & cancer 50, 859 (November, 2011).
32. A. Lujambio et al., Genetic unmasking of an epigenetically silenced microRNA in human cancer cells. Cancer research 67, 1424 (Feb. 15, 2007).

Without further elaboration, it is believed that one skilled in the art can, based on the above description, utilize the present invention to its fullest extent. The specific embodiments are, therefore, to be construed as merely illustrative, and not limitative of the remainder of the disclosure in any way whatsoever. All publications cited herein are incorporated by reference for the purposes or subject matter referenced herein.

The indefinite articles “a” and “an,” as used herein in the specification and in the claims, unless clearly indicated to the contrary, should be understood to mean “at least one.”

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A method, comprising:
a) analyzing genomic DNA from a canine subject for the presence of a single nucleotide polymorphism (SNP) selected from:
i) one or more chromosome 1 SNPs,
ii) one or more chromosome 2 SNPs,
iii) one or more chromosome 3 SNPs,
iv) one or more chromosome 5 SNPs,
v) one or more chromosome 7 SNPs,
vi) one or more chromosome 8 SNPs,
vii) one or more chromosome 9 SNPs,
viii) one or more chromosome 11 SNPs,
ix) one or more chromosome 13 SNPs,
x) one or more chromosome 14 SNPs,
xi) one or more chromosome 15 SNPs,
xii) one or more chromosome 16 SNPs,
xii) one or more chromosome 17 SNPs,
xiv) one or more chromosome 18 SNPs,
xv) one or more chromosome 19 SNPs,
xvi) one or more chromosome 21 SNPs,
xvii) one or more chromosome 25 SNPs,
xvii) one or more chromosome 26 SNPs,
xix) one or more chromosome 32 SNPs,
xx) one or more chromosome 35 SNPs,
xxi) one or more chromosome 36 SNPs, and
xxii) one or more chromosome 38 SNPs; and
b) identifying a canine subject having the SNP as a subject at elevated risk of developing osteosarcoma or having an undiagnosed osteosarcoma.

2. The method of claim 1, wherein the SNP is selected from BICF2P133066, BICF2P1421479, BICF2S2308696, BICF2P508906, BICF2P508905, BICF2S23216058, BICF2S23216058, BICF2P266591, BICF2P1332375, BICF2S23231062, BICF2S22945043, BICF2P326880, BICF2P893664, BICF2P1420547, BICF2P698281, BICF2S22919383, BICF2S22947803, BICF2S22947803, BICF2S22959094, BICF2S23228287, BICF2S23036972, BICF2P51623, BICF2P1346510, BICF2P1323908, BICF2P1137984, BICF2P1115364, BICF2P58266, BICF2P627162, BICF2P1422910, BICF2P162782, BICF2P162782, BICF2P1342901, BICF2P868731, BICF2P768889, BICF2P1052528, BICF2P408119, BICF2P1468011, BICF2P219326, BICF2P1462759, BICF2P307386, BICF2P1010170, BICF2S23038485, BICF2G630672865, BICF2G630672813, BICF2P1369145, BICF2G630672770, BICF2P81989, BICF2P916235, BICF2G630672753, BICF2P1177075, BICF2P411325, BICF2P1210630, TIGRP2P407733, BICF2P341331, BICF2P318350, BICF2S2335735, BICF2P1003572, BICF2P1104551, BICF2S23550277, BICF2P870378, BICF2P866460, BICF2P1303772, BICF2S23738710, BICF2P344455, BICF2P825177, BICF2S23324500, BICF2S23544574, BICF2P119783, BICF2S23758510, BICF2S23724888, BICF2P1129874, BICF2S23535303, BICF2S23520119, G326F32S322, BICF2S23238674, BICF2P645758, BICF2P189890, BICF2P819174, BICF2P162666, BICF2P1366853, BICF2P775251, BICF2S23746532, BICF2P1162557, BICF2S23538747, BICF2S23538670, BICF2S23218055, BICF2P680751, BICF2S23510137, BICF2P849639, BICF2S22945333, BICF2S2298851, TIGRP2P238123, TIGRP2P238132, BICF2P1466354, BICF2P440326, BICF2P874005, BICF2P928021, BICF2P1182592, BICF2P1378069, TIGRP2P238162, TIGRP2P253880, BICF2P461252, BICF2P879737, BICF2P163146, BICF2S23259485, TIGRP2P253975, BICF2S23760612, TIGRP2P254013, TIGRP2P254028, BICF2S23750273, BICF2P228579, TIGRP2P254054, BICF2P531896, TIGRP2P254060, BICF2P766570, BICF2P1014267, BICF2P1006929, BICF2P1299781, BICF2P672676, BICF2S23761559, BICF2P15617, BICF2P439160, TIGRP2P254095, TIGRP2P254109, BICF2P477812, BICF2P1238318, BICF2P1354921, BICF2S23741435, BICF2P37118, TIGRP2P254175, BICF2P1123483, TIGRP2P254184, BICF2P825842, BICF2P243632, BICF2P1139856, BICF2P1376844, TIGRP2P254212, TIGRP2P254216, and TIGRP2P254223.

3. The method of claim 1, wherein the SNP is selected from BICF2P133066, BICF2S2308696, BICF2P508906, BICF2P508905, BICF2S23216058, BICF2S23216058, BICF2P266591, BICF2P1332375, BICF2S23231062, BICF2S22945043, BICF2P326880, BICF2P893664, BICF2P1420547, BICF2P698281, BICF2S22919383, BICF2S22947803, BICF2S22947803, BICF2S22959094, BICF2S23228287, BICF2S23036972, BICF2P51623, BICF2P1346510, BICF2P1323908, BICF2P1137984, BICF2P1115364, BICF2P58266, BICF2P627162, BICF2P1422910, BICF2P162782, BICF2P162782, BICF2P1342901, BICF2P868731, BICF2P768889, BICF2P1052528, BICF2P408119, BICF2P1468011, BICF2P219326, BICF2P1462759, BICF2P307386, BICF2P1010170, BICF2P229090, BICF2S23516022, and BICF2S22922837.

4. The method of claim 1, wherein the SNP is BICF2P133066.

5. The method of any one of claims 1 to 4, wherein the genomic DNA is obtained from a bodily fluid or tissue sample of the subject.

6. The method of claim 5, wherein the genomic DNA is obtained from a blood or saliva sample of the subject.

7. The method of any one of claims 1 to 6 wherein the genomic DNA is analyzed using a single nucleotide polymorphism (SNP) array.

8. The method of any one of claims 1 to 6 wherein the genomic DNA is analyzed using a bead array.

9. The method of any one of claims 1 to 6 wherein the genomic DNA is analyzed using a nucleic acid sequencing assay.

10. The method of claim 1, wherein the SNP is two or more SNPs.

11. The method of claim 1, wherein the SNP is three or more SNPs.

12. A method, comprising:
(a) analyzing genomic DNA from a canine subject for the presence of a risk haplotype selected from:
a risk haplotype having chromosome coordinates chr11:44392734-44414985,
a risk haplotype having chromosome coordinates chr8:35433142-35454649,
a risk haplotype having chromosome coordinates chr13:14549973-14645634,
a risk haplotype having chromosome coordinates chr25:21831580-21921256,
a risk haplotype having chromosome coordinates chr14:48831824-49203827,
a risk haplotype having chromosome coordinates chr5:16071171-16152955,
a risk haplotype having chromosome coordinates chr19:33963105-34145310,
a risk haplotype having chromosome coordinates chr16:43665149-43737129,
a risk haplotype having chromosome coordinates chr15:63767963-63800415,
a risk haplotype having chromosome coordinates chr16:40883517-41081510,
a risk haplotype having chromosome coordinates chr25:43476429-43528145,
a risk haplotype having chromosome coordinates chr1:112977233-113081800,
a risk haplotype having chromosome coordinates chr3:5162058-6465753,
a risk haplotype having chromosome coordinates chr7:64631053-64703475,
a risk haplotype having chromosome coordinates chr1:115582915-116790630,
a risk haplotype having chromosome coordinates chr2:19212450-19542015,
a risk haplotype having chromosome coordinates chr1:122033806-122051988,
a risk haplotype having chromosome coordinates chr35:18326079-18345318,
a risk haplotype having chromosome coordinates chr9:47647012-47668054,
a risk haplotype having chromosome coordinates chr38:11252518-11739329,
a risk haplotype having chromosome coordinates chr21:46231985-46363479,
a risk haplotype having chromosome coordinates chr17:14465884-14482152,
a risk haplotype having chromosome coordinates chr32:25136302-25156153,
a risk haplotype having chromosome coordinates chr36:29637804-29663408,
a risk haplotype having chromosome coordinates chr15:37986345-39974762,
a risk haplotype having chromosome coordinates chr1:29405587-29914411,
a risk haplotype having chromosome coordinates chr26:32374093-32428448,
a risk haplotype having chromosome coordinates chr25:29658978-29767164,
a risk haplotype having chromosome coordinates chr26:3529343-3550075,
a risk haplotype having chromosome coordinates chr5:14720254-15466603,
a risk haplotype having chromosome coordinates chr18:4266743-5854451,
a risk haplotype having chromosome coordinates chr1:16768869-18150476,
a risk haplotype having chromosome coordinates chr9:18896060-19633155, and
a risk haplotype having chromosome coordinates chr11:44390633-44406002; and
(b) identifying a canine subject having the mutation as a subject at elevated risk of developing osteosarcoma or having an undiagnosed osteosarcoma.

13. The method of claim 12, wherein the risk haplotype is selected from:
a risk haplotype having chromosome coordinates chr11:44392734-44414985,
a risk haplotype having chromosome coordinates chr8:35433142-35454649,
a risk haplotype having chromosome coordinates chr1:115582915-116790630,
a risk haplotype having chromosome coordinates chr2:19212450-19542015,
a risk haplotype having chromosome coordinates chr1:122033806-122051988,
a risk haplotype having chromosome coordinates chr35:18326079-18345318,
a risk haplotype having chromosome coordinates chr9:47647012-47668054,
a risk haplotype having chromosome coordinates chr38:11252518-11739329,
a risk haplotype having chromosome coordinates chr5:14720254-15466603, and
a risk haplotype having chromosome coordinates chr18:4266743-5854451.

14. The method of claim 12, wherein the risk haplotype is selected from:
a risk haplotype having chromosome coordinates chr11:44392734-44414985,
a risk haplotype having chromosome coordinates chr1:115582915-116790630, and
a risk haplotype having chromosome coordinates chr5:14720254-15466603.

15. The method of claim 12, wherein the risk haplotype is the risk haplotype having chromosome coordinates chr11:44392734-44414985.

16. The method of any one of claims 12 to 15, wherein the presence of the risk haplotype is detected by analyzing the genomic DNA for the presence of a SNP.

17. The method of any one of claims 12 to 16, wherein the genomic DNA is obtained from a bodily fluid or tissue sample of the subject.

18. The method of claim 17, wherein the genomic DNA is obtained from a blood or saliva sample of the subject.

19. The method of any one of claims 12 to 18, wherein the genomic DNA is analyzed using a single nucleotide polymorphism (SNP) array.

20. The method of any one of claims 12 to 18, wherein the genomic DNA is analyzed using a bead array.

21. The method of any one of claims 12 to 18, wherein the genomic DNA is analyzed using a nucleic acid sequencing assay.

22. The method of claim 12, wherein the mutation is two or more mutations.

23. The method of claim 12, wherein the mutation is three or more mutations.

24. The method of claim 12, wherein the genomic region is two or more genomic regions.

25. The method of claim 12, wherein the genomic region is three or more genomic regions.

26. A method, comprising:
(a) analyzing genomic DNA from a canine subject for the presence of a mutation in a gene selected from:
one or more genes located within a risk haplotype having chromosome coordinates chr11:44392734-44414985,
one or more genes located within a risk haplotype having chromosome coordinates chr8:35433142-35454649,
one or more genes locates within a risk haplotype having chromosome coordinates chr13: 14549973-14645634,
one or more genes located within a risk haplotype having chromosome coordinates chr25:21831580-21921256,
one or more genes located within a risk haplotype having chromosome coordinates chr14:48831824-49203827,
one or more genes located within a risk haplotype having chromosome coordinates chr5:16071171-16152955,
one or more genes located within a risk haplotype having chromosome coordinates chr19:33963105-34145310,
one or more genes located within a risk haplotype having chromosome coordinates chr16:43665149-43737129,
one or more genes located within a risk haplotype having chromosome coordinates chr15:63767963-63800415,
one or more genes located within a risk haplotype having chromosome coordinates chr16:40883517-41081510,
one or more genes located within a risk haplotype having chromosome coordinates chr25:43476429-43528145,
one or more genes located within a risk haplotype having chromosome coordinates chr1:112977233-113081800,
one or more genes located within a risk haplotype having chromosome coordinates chr3:5162058-6465753,
one or more genes located within a risk haplotype having chromosome coordinates chr7:64631053-64703475,
one or more genes located within a risk haplotype having chromosome coordinates chr1:115582915-116790630,
one or more genes located within a risk haplotype having chromosome coordinates chr2:19212450-19542015,
one or more genes located within a risk haplotype having chromosome coordinates chr1:122033806-122051988,
one or more genes located within a risk haplotype having chromosome coordinates chr35: 18326079-18345318,
one or more genes located within a risk haplotype having chromosome coordinates chr9:47647012-47668054,
one or more genes located within a risk haplotype having chromosome coordinates chr38: 11252518-11739329,
one or more genes located within a risk haplotype having chromosome coordinates chr21:46231985-46363479,
one or more genes located within a risk haplotype having chromosome coordinates chr17: 14465884-14482152,
one or more genes located within a risk haplotype having chromosome coordinates chr32:25136302-25156153,
one or more genes located within a risk haplotype having chromosome coordinates chr36:29637804-29663408,
one or more genes located within a risk haplotype having chromosome coordinates chr15:37986345-39974762,
one or more genes located within a risk haplotype having chromosome coordinates chr1:29405587-29914411,
one or more genes located within a risk haplotype having chromosome coordinates chr26: 32374093-32428448,
one or more genes located within a risk haplotype having chromosome coordinates chr25:29658978-29767164,
one or more genes located within a risk haplotype having chromosome coordinates chr26:3529343-3550075,
one or more genes located within a risk haplotype having chromosome coordinates chr5:14720254-15466603,
one or more genes located within a risk haplotype having chromosome coordinates chr18:4266743-5854451,
one or more genes located within a risk haplotype having chromosome coordinates chr1:16768869-18150476,
one or more genes located within a risk haplotype having chromosome coordinates chr9:18896060-19633155, and
one or more genes located within a risk haplotype having chromosome coordinates chr11:44390633-44406002; and
(b) identifying a canine subject having the mutation as a subject at elevated risk of developing osteosarcoma or having an undiagnosed osteosarcoma.

27. The method of claim 26, wherein the gene is selected from:
one or more genes located within a risk haplotype having chromosome coordinates chr11:44392734-44414985,
one or more genes located within a risk haplotype having chromosome coordinates chr8:35433142-35454649,
one or more genes located within a risk haplotype having chromosome coordinates chr1:115582915-116790630,
one or more genes located within a risk haplotype having chromosome coordinates chr2:19212450-19542015,
one or more genes located within a risk haplotype having chromosome coordinates chr1:122033806-122051988,
one or more genes located within a risk haplotype having chromosome coordinates chr35:18326079-18345318,
one or more genes located within a risk haplotype having chromosome coordinates chr9:47647012-47668054,
one or more genes located within a risk haplotype having chromosome coordinates chr38:11252518-11739329,
one or more genes located within a risk haplotype having chromosome coordinates chr5:14720254-15466603, and
one or more genes located within a risk haplotype having chromosome coordinates chr18:4266743-5854451.

28. The method of claim 26, wherein the gene is selected from:
one or more genes located within a risk haplotype having chromosome coordinates chr11:44392734-44414985,
one or more genes located within a risk haplotype having chromosome coordinates chr1:115582915-116790630, and
one or more genes located within a risk haplotype having chromosome coordinates chr5:14720254-15466603.

29. The method of claim 26, wherein the gene is one or more genes located within the risk haplotype having chromosome coordinates chr11:44392734-44414985.

30. The method of claim 26, wherein the gene is selected from CDKN2B-AS, OTX2, BMPER, GRIK4, EN1, MARCO, MTMR7, SGCZ, CCL20, CD3EAP, ERCC1, ERCC2, FOSB, PPP1R13L, FER, MAN2A1, PJA2, CHST9, ADCK4, AKT2, AXL, BLVRB, C19orf47, C19orf54, CNTD2, CYP2A7, CYP2B6, CYP2S1, DLL3, EGLN2, FBL, FCGBP, GMFG, HIPK4, HNRNPUL1, ITPKC, LEUTX, LTBP4, MAP3K10, MED29, NUMBL, PLD3, PLEKHG2, PSMC4, RAB4B, SAMD4B, SERTAD1, SERTAD3, SHKBP1, SNRPA, SPTBN4, SUPT5H, TIMM50, KIAA1462, Cl9orf40, CEP89, RHPN2, BLMH, TMIGD1, FAM5C, NELL1, EMCN, AMDHD1, CCDC38, CDK17, ELK3, FGD6, HAL, LTA4H, METAP2, NDUFA12, NEDD1, NR2C1, NTN4, SNRPF, USP44,VEZT, EYA4, TCF21, ARVCF, C22orf25, COMT, XKR6, FBRSL1, BLID, C7orf72, COBL, DDC, FIGNL1, GRB10, IKZF1, VWC2, ZPBP, BCL2, KIAA1468, PHLPP1, PIGN, RNF152, TNFRSF11A, ZCCHC2, ABCA5, KCNJ16, KCNJ2, MAP2K6, CDKN2A, and CDKN2B.

31. The method of claim 26, wherein the gene is selected from CDKN2B-AS, OTX2, BMPER, EN1, DLL3, KIAA1462, FAM5C, NELL1, EMCN, TCF21, BLID, VWC2, BCL2, and TNFRSF11A.

32. The method of claim 26, wherein the gene is selected from CDKN2B-AS, OTX2, ADCK4, AKT2, AXL, BLVRB, C19orf47, C19orf54, CNTD2, CYP2A7, CYP2B6, CYP2S1, DLL3, EGLN2, FBL, FCGBP, GMFG, HIPK4, HNRNPUL1, ITPKC, LEUTX, LTBP4, MAP3K10, MED29, NUMBL, PLD3, PLEKHG2, PSMC4, RAB4B, SAMD4B, SERTAD1, SERTAD3, SHKBP1, SNRPA, SPTBN4, SUPT5H, TIMM50, KIAA1462, Cl9orf40, CEP89, RHPN2, BLMH, TMIGD1, FAM5C, BLID, C7orf72, COBL, DDC, FIGNL1, GRB10, IKZF1, VWC2, and ZPBP.

33. The method of claim 26, wherein the gene is selected from CDKN2B-AS, ADCK4, AKT2, AXL, BLVRB, Cl9orf47,C19orf54, CNTD2, CYP2A7, CYP2B6, CYP2S1, DLL3, EGLN2, FBL, FCGBP, GMFG, HIPK4, HNRNPUL1, ITPKC, LEUTX, LTBP4, MAP3K10, MED29, NUMBL, PLD3, PLEKHG2, PSMC4, RAB4B, SAMD4B, SERTAD1, SERTAD3, SHKBP1, SNRPA, SPTBN4, SUPT5H, TIMM50, and BLID.

34. The method of claim 26, wherein the gene is selected from CDKN2B-AS, CDKN2A, and CDKN2B.

35. The method of any one of claims 26 to 34, wherein the genomic DNA is obtained from a bodily fluid or tissue sample of the subject.

36. The method of claim 35, wherein the genomic DNA is obtained from a blood or saliva sample of the subject.

37. The method of any one of claims 26 to 36, wherein the genomic DNA is analyzed using a single nucleotide polymorphism (SNP) array.

38. The method of any one of claims 26 to 36, wherein the genomic DNA is analyzed using a bead array.

39. The method of any one of claims 26 to 36, wherein the genomic DNA is analyzed using a nucleic acid sequencing assay.

40. The method of claim 26, wherein the mutation is two or more mutations.

41. The method of claim 26, wherein the mutation is three or more mutations.

42. The method of claim 26, wherein the gene is two or more genes.

43. The method of claim 26, wherein the gene is three or more genes.

44. A method, comprising:
(a) analyzing genomic DNA in a sample from a subject for presence of a mutation in a gene selected from:
one or more genes located within a risk haplotype having chromosome coordinates chr11:44392734-44414985 or an orthologue of such a gene,
one or more genes located within a risk haplotype having chromosome coordinates chr8:35433142-35454649 or an orthologue of such a gene,
one or more genes located within a risk haplotype having chromosome coordinates chr13:14549973-14645634 or an orthologue of such a gene,
one or more genes located within a risk haplotype having chromosome coordinates chr25:21831580-21921256 or an orthologue of such a gene,
one or more genes located within a risk haplotype having chromosome coordinates chr14:48831824-49203827 or an orthologue of such a gene,
one or more genes located within a risk haplotype having chromosome coordinates chr5:16071171-16152955 or an orthologue of such a gene,
one or more genes located within a risk haplotype having chromosome coordinates chr19:33963105-34145310 or an orthologue of such a gene,
one or more genes located within a risk haplotype having chromosome coordinates chr16:43665149-43737129 or an orthologue of such a gene,
one or more genes located within a risk haplotype having chromosome coordinates chr15:63767963-63800415 or an orthologue of such a gene,
one or more genes located within a risk haplotype having chromosome coordinates chr16:40883517-41081510 or an orthologue of such a gene,
one or more genes located within a risk haplotype having chromosome coordinates chr25:43476429-43528145 or an orthologue of such a gene,
one or more genes located within a risk haplotype having chromosome coordinates chr1:112977233-113081800 or an orthologue of such a gene,
one or more genes located within a risk haplotype having chromosome coordinates chr3:5162058-6465753 or an orthologue of such a gene,
one or more genes located within a risk haplotype having chromosome coordinates chr7:64631053-64703475 or an orthologue of such a gene,
one or more genes located within a risk haplotype having chromosome coordinates chr1:115582915-116790630 or an orthologue of such a gene,
one or more genes located within a risk haplotype having chromosome coordinates chr2:19212450-19542015 or an orthologue of such a gene,
one or more genes located within a risk haplotype having chromosome coordinates chr1:122033806-122051988 or an orthologue of such a gene,
one or more genes located within a risk haplotype having chromosome coordinates chr35:18326079-18345318 or an orthologue of such a gene,
one or more genes located within a risk haplotype having chromosome coordinates chr9:47647012-47668054 or an orthologue of such a gene,
one or more genes located within a risk haplotype having chromosome coordinates chr38:11252518-11739329 or an orthologue of such a gene,
one or more genes located within a risk haplotype having chromosome coordinates chr21:46231985-46363479 or an orthologue of such a gene,
one or more genes located within a risk haplotype having chromosome coordinates chr17:14465884-14482152 or an orthologue of such a gene,
one or more genes located within a risk haplotype having chromosome coordinates chr32:25136302-25156153 or an orthologue of such a gene,
one or more genes located within a risk haplotype having chromosome coordinates chr36:29637804-29663408 or an orthologue of such a gene,
one or more genes located within a risk haplotype having chromosome coordinates chr15:37986345-39974762 or an orthologue of such a gene,
one or more genes located within a risk haplotype having chromosome coordinates chr1:29405587-29914411 or an orthologue of such a gene,
one or more genes located within a risk haplotype having chromosome coordinates chr26:32374093-32428448 or an orthologue of such a gene,
one or more genes located within a risk haplotype having chromosome coordinates chr25:29658978-29767164 or an orthologue of such a gene,
one or more genes located within a risk haplotype having chromosome coordinates chr26:3529343-3550075 or an orthologue of such a gene,
one or more genes located within a risk haplotype having chromosome coordinates chr5:14720254-15466603 or an orthologue of such a gene,
one or more genes located within a risk haplotype having chromosome coordinates chr18:4266743-5854451 or an orthologue of such a gene,
one or more genes located within a risk haplotype having chromosome coordinates chr1:16768869-18150476 or an orthologue of such a gene,
one or more genes located within a risk haplotype having chromosome coordinates chr9:18896060-19633155 or an orthologue of such a gene, and
one or more genes located within a risk haplotype having chromosome coordinates chr11:44,390,633-44,406,002 or an orthologue of such a gene; and
(b) identifying a subject having the mutation as a subject at elevated risk of developing osteosarcoma or having an undiagnosed osteosarcoma.

45. The method of claim 44, wherein the subject is a human subject.

46. The method of claim 44, wherein the subject is a canine subject.

47. The method of any one of claims 44 to 46, wherein the genomic DNA is obtained from a bodily fluid or tissue sample of the subject.

48. The method of claim 47, wherein the genomic DNA is obtained from a blood or saliva sample of the subject.

49. The method of any one of claims 44 to 48, wherein the genomic DNA is analyzed using a single nucleotide polymorphism (SNP) array.

50. The method of any one of claims 44 to 48, wherein the genomic DNA is analyzed using a bead array.

51. The method of any one of claims 44 to 48, wherein the genomic DNA is analyzed using a nucleic acid sequencing assay.

52. The method of claim 44, wherein the gene is two or more genes.

53. The method of claim 44, wherein the gene is three or more genes.

54. The method of claim 44, wherein the mutation is two or more mutations.

55. The method of claim 44, wherein the mutation is three or more mutations.
